(19) United States
(12) Patent Application Publication
(10) Pub. No.: US 2020/0139146 A1
(43) Pub. Date: May 7, 2020

(54) APPARATUS AND METHOD FOR TREATING TEETH

(71) Applicant: SONENDO, INC., Laguna Hills, CA (US)

(72) Inventors: Mehrzad Khakpour, Laguna Hills, CA (US); Bjarne Bergheim, Mission Viejo, CA (US); Jon Jacobson, Irvine, CA (US); Richard Cook, North Tustin, CA (US); Scott William Thomas, Trabuco Canyon, CA (US); Ramtin Malool, Laguna Niguel, CA (US)

(21) Appl. No.: 16/582,864

(22) Filed: Sep. 25, 2019

Related U.S. Application Data
(60) Provisional application No. 62/736,119, filed on Sep. 25, 2018.

(51) Int. Cl. A61N 2/00; A61C 5/50; A61N 2/02

(52) U.S. Cl. CPC A61N 2/002; A61N 2/02; A61N 2/004; A61C 5/50

(57) ABSTRACT

Examples of apparatus, methods, and compositions for endodontic treatments are described. In certain implementations, the apparatus can include a treatment fluid supply configured to supply a treatment fluid to a treatment region of the tooth and a pressure wave generator configured to generate pressure waves in the treatment fluid. The pressure wave generator can include a volume housing an electromagnetically responsive material, a diaphragm including a first side and a second side, the first side exposed to the volume, the second side exposed to the treatment fluid, the diaphragm being movable such that movement of the electromagnetically responsive material within the volume causes movement of the diaphragm, and an electromagnetic generator coupled to the volume, the electromagnetic generator configured to generate electromagnetic energy. The electromagnetically responsive material can be responsive to the electromagnetic energy generated by the electromagnetic generator so as to cause the movement of the diaphragm.

### CROSS-REFERENCE TO RELATED APPLICATIONS

This application claims priority to U.S. Provisional Patent Application No. 62/736,119 filed Sep. 25, 2018, the contents of which are incorporated by reference herein in their entirety and for all purposes.

### BACKGROUND

### Field

The present disclosure relates generally to dentistry and endodontics and to apparatus, methods, and compositions for treating a tooth.

### Description of the Related Art

In conventional dental and endodontic procedures, mechanical instruments such as drills, files, brushes, etc. are used to clean unhealthy material from a tooth. For example, dentists often use drills to mechanically break up carious regions (e.g., cavities) in a surface of the tooth. Such procedures are often painful for the patient and frequently do not remove all the diseased material. Furthermore, in conventional root canal treatments, an opening is drilled through the crown of a diseased tooth, and endodontic files are inserted into the root canal system to open the canal spaces and remove organic material therein. The root canal is then filled with solid matter such as gutta percha or a flowable obturation material, and the tooth is restored. However, this procedure will not remove all organic material from the canal spaces, which can lead to post-procedure complications such as infection. In addition, motion of the endodontic file and/or other sources of positive pressure may force organic material through an apical opening into periapical tissues. In some cases, an end of the endodontic file itself may pass through the apical opening. Such events may result in trauma to the soft tissue near the apical opening and lead to post-procedure complications. Accordingly, there is a continuing need for improved dental and endodontic treatments.

### SUMMARY

Various non-limiting aspects of the present disclosure will now be provided to illustrate features of the disclosed apparatus, methods, and compositions. Examples of apparatus, methods, and compositions for endodontic treatments are provided.

In one embodiment, an apparatus for treating a tooth is disclosed. The apparatus can comprise a treatment fluid supply configured to supply a treatment fluid to a treatment region of the tooth and a pressure wave generator configured to generate pressure waves in the treatment fluid. The pressure wave generator can comprise a volume housing an electromagnetically responsive material, a diaphragm comprising a first side and a second side, the first side exposed to the volume, the second side exposed to the treatment fluid, the diaphragm being movable such that movement of the electromagnetically responsive material within the volume causes movement of the diaphragm, and an electromagnetic generator coupled to the volume, the electromagnetic generator configured to generate electromagnetic energy. The electromagnetically responsive material can be responsive to the electromagnetic energy generated by the electromagnetic generator so as to cause the movement of the diaphragm.

In certain implementations, the electromagnetically responsive material comprises a ferrofluid. In certain implementations, the electromagnetic generator comprises a magnetic field generator. In certain implementations, the electromagnetic generator comprises a coiled wire. In certain implementations, the volume housing the electromagnetically responsive material can be located within a diameter of the coiled wire. In certain implementations, the treatment fluid supply extends through a diameter of the coiled wire. In certain implementations, the electromagnetic generator can be positioned within a flow path of the treatment fluid within the treatment fluid supply. In certain implementations, the electromagnetic generator can be isolated from a flow path of the treatment fluid within the treatment fluid supply. In certain implementations, the apparatus can further comprise a deformable material separating the volume housing the electromagnetically responsive material and the electromagnetic generator. In certain implementations, the electromagnetically responsive material can be responsive to the electromagnetic energy generated by the electromagnetic generator so as to generate pressure waves in the treatment fluid. In certain implementations, the apparatus further comprises a controller configured to control operation of the electromagnetic generator. In certain implementations, the controller can be configured to send control signals to the electromagnetic generator, the control signals selected to cause the electromagnetic generator to generate electromagnetic energy which causes a response in the electromagnetically responsive material to produce acoustic waves in the treatment fluid having a predetermined acoustic signature. In certain implementations, the apparatus further comprises a fluid platform comprising a chamber to be positioned against the tooth, the chamber shaped to retain treatment fluid. In certain implementations, the pressure wave generator can be exposed to the chamber. In certain implementations, the apparatus further comprises a fluid passage in fluid communication with the chamber via an opening at a distal end of the fluid passage, the fluid passage configured to deliver treatment fluid to the chamber through the opening. In certain implementations, the pressure wave generator can be positioned to generate pressure waves in the treatment fluid at a location within the fluid passage proximal of the opening and outside of the tooth, the generated pressure waves being transmitted through the treatment fluid in the fluid passage and the chamber to the treatment region of the tooth. In certain implementations, the apparatus further comprises a fluid motion generator configured to generate bulk fluid motion in the treatment fluid. In certain implementations, the pressure wave generator can be configured to generate bulk fluid motion in the treatment fluid.

In another embodiment, an apparatus for treating a tooth is disclosed. The apparatus can comprise a pressure wave generator comprising an electromagnetic element configured to convert electromagnetic energy to acoustic waves in a fluid by way of an electromagnetically responsive medium and a controller configured to send control signals to the electromagnetic element, the control signals selected to cause the electromagnetic element to generate electromagnetic energy that produces acoustic waves in the fluid having a predetermined acoustic signature.

In certain implementations, the electromagnetic element comprises a magnetic field generator. In certain implementations, the magnetic field generator comprises a conductor. The controller can be configured to generate a current in the conductor so that the magnetic field generator creates a corresponding changing magnetic field at one or a plurality of frequencies and/or pulsation patterns that correspond to the predetermined acoustic signature. In certain implementations, the conductor comprises a coiled wire. In certain implementations, the electromagnetically responsive medium comprises a plurality of ferrous particles. In certain implementations, the plurality of ferrous particles are suspended within the fluid. In certain implementations, the electromagnetic element can be configured to generate electromagnetic energy that causes movement of the ferrous particles in the fluid which produces waves in the fluid having the predetermined acoustic signature. In certain implementations, the apparatus further comprises a volume housing the electromagnetically responsive medium and a diaphragm comprising a first side and a second side, the first side exposed to the volume, the second side exposed to the fluid, the diaphragm being movable such that movement of the electromagnetically responsive medium within the volume causes movement of the diaphragm. In certain implementations, the electromagnetically responsive medium comprise a dielectric that separates two conductive plates. In certain implementations, the electromagnetically responsive medium comprises one or more magnets. In certain implementations, the apparatus further comprises a fluid platform comprising a chamber to be positioned against the tooth, the chamber shaped to retain the fluid. In certain implementations, the pressure wave generator can be exposed to the chamber. In certain implementations, the apparatus further comprises a fluid passage in fluid communication with the chamber via an opening at a distal end of the fluid passage, the fluid passage configured to deliver the fluid to the chamber through the opening. In certain implementations, the pressure wave generator can be positioned to generate pressure waves in the fluid at a location within the fluid passage proximal of the opening and outside of the tooth, the generated pressure waves being transmitted through the treatment fluid in the fluid passage and the chamber to a treatment region of the tooth. In certain implementations, the apparatus further comprises a fluid motion generator configured to generate bulk fluid motion in the treatment fluid. In certain implementations, the pressure wave generator can be configured to generate bulk fluid motion in the treatment fluid. In certain implementations, the apparatus further comprises a sensor configured to measure an acoustic signature of acoustic waves produced during a treatment procedure. In certain implementations, the controller can be configured to adjust the control signals based on the measured acoustic signature of the acoustic waves produced during the treatment procedure. In certain implementations, the controller can be configured to compare the measured acoustic signature to the predetermined acoustic signature and adjust the control signals based on the comparison between the measured acoustic signature and the predetermined acoustic signature.

In yet another embodiment, an apparatus for treating a tooth is disclosed. The apparatus can comprise a pressure wave generator comprising an electromagnetic element configured to convert electromagnetic energy to acoustic waves in a fluid, the electromagnetic element comprising a coiled wire configured to generate a magnetic field.

In certain implementations, the electromagnetic element can be positioned within a flow path of the fluid. In certain implementations, the electromagnetic element can be isolated from a flow path of the fluid. In certain implementations, the apparatus further comprises a controller configured to control operation of the electromagnetic element. In certain implementations, the controller can be configured to send control signals to the electromagnetic element, the control signals selected to cause the electromagnetic element to convert electromagnetic energy to acoustic waves having a predetermined acoustic signature. In certain implementations, the fluid comprises a treatment fluid and an electromagnetically responsive material, the electromagnetically responsive material being responsive to the electromagnetic energy generated by the electromagnetic element. In certain implementations, the electromagnetically responsive material comprises a plurality of ferrous particles. In certain implementations, the electromagnetic element can be configured to convert electromagnetic energy to acoustic waves in the fluid by causing movement of the ferrous particles in the fluid. In certain implementations, the pressure wave generator further comprises a volume housing an electromagnetically responsive medium and a diaphragm comprising a first side and a second side, the first side exposed to the volume, the second side exposed to the fluid, the diaphragm being movable such that movement of the electromagnetically responsive medium within the volume causes movement of the diaphragm. In certain implementations, the volume housing the electromagnetically responsive material can be located within a diameter of the coiled wire. In certain implementations, the apparatus further comprises a deformable material separating the volume housing the electromagnetically responsive material and the electromagnetic element. In certain implementations, the apparatus further comprises a fluid platform comprising a chamber to be positioned against the tooth, the chamber shaped to retain the fluid. In certain implementations, the pressure wave generator can be exposed to the chamber. In certain implementations, the apparatus further comprises a fluid passage in fluid communication with the chamber via an opening at a distal end of the fluid passage, the fluid passage configured to deliver treatment fluid to the chamber through the opening. In certain implementations, the pressure wave generator can be positioned to generate acoustic waves in the treatment fluid at a location within the fluid passage proximal of the opening and outside of the tooth, the generated acoustic waves being transmitted through the treatment fluid in the fluid passage and the chamber to the treatment region of the tooth. In certain implementations, the apparatus further comprises a fluid motion generator configured to generate bulk fluid motion in the treatment fluid. In certain implementations, the pressure wave generator can be configured to generate bulk fluid motion in the treatment fluid.

In yet another embodiment, an apparatus for treating a tooth is disclosed. The apparatus can comprise a pressure wave generator comprising a volume to be filled with an electromagnetically responsive material a diaphragm forming at least a portion of the volume to contain the electromagnetically responsive material in the volume, the diaphragm being movable such that movement of the electromagnetically responsive material within the volume causes movement of the diaphragm.

In certain implementations, the diaphragm has a first side configured to contact the electromagnetically responsive material during the treatment procedure and a second side configured to contact a treatment fluid at a treatment region of the tooth during the treatment procedure. In certain implementations, the electromagnetically responsive material comprises a ferrofluid. In certain implementations, the pressure wave generator further comprises an electromagnetic generator configured to generate electromagnetic energy in the electromagnetically responsive material. In certain implementations, the electromagnetic generator comprises a magnetic field generator. In certain implementations, the electromagnetic generator comprises a coiled wire. In certain implementations, the volume housing the electromagnetically responsive material can be located within a diameter of the coiled wire. In certain implementations, the electromagnetic generator can be positioned within a flow path of a treatment fluid. In certain implementations, the electromagnetic generator can be isolated from a flow path of a treatment fluid. In certain implementations, the apparatus further comprises a deformable material separating the volume housing the electromagnetically responsive material and the electromagnetic generator. In certain implementations, the electromagnetically responsive material can be responsive to the electromagnetic energy generated by the electromagnetic generator so as to generate pressure waves in a treatment fluid. In certain implementations, the apparatus further comprises a controller configured to control operation of the electromagnetic generator. In certain implementations, the controller can be configured to send control signals to the electromagnetic generator, the control signals selected to cause the electromagnetic generator to generate electromagnetic energy which causes a response in the electromagnetically responsive material to produce acoustic waves in the treatment fluid having a predetermined acoustic signature. In certain implementations, the apparatus further comprises a fluid platform comprising a chamber to be positioned against the tooth, the chamber shaped to retain a treatment fluid. In certain implementations, the pressure wave generator can be exposed to the chamber. In certain implementations, the apparatus further comprises a fluid passage in fluid communication with the chamber via an opening at a distal end of the fluid passage, the fluid passage configured to deliver treatment fluid to the chamber through the opening. In certain implementations, the pressure wave generator can be positioned to generate pressure waves in the treatment fluid at a location within the fluid passage proximal of the opening and outside of the tooth, the generated pressure waves being transmitted through the treatment fluid in the fluid passage and the chamber to the treatment region of the tooth. In certain implementations, the apparatus further comprises a fluid motion generator configured to generate bulk fluid motion to the treatment fluid. In certain implementations, the pressure wave generator can be configured to generate bulk fluid motion in the treatment region.

In yet another embodiment, an apparatus for treating a tooth is disclosed. The apparatus can comprise a fluid supply configured to supply a fluid to a treatment region of the tooth, the fluid comprising a treatment fluid and an electromagnetically responsive material and a pressure wave generator configured to generate pressure waves in the treatment fluid. The pressure wave generator can comprise an electromagnetic generator configured to generate electromagnetic energy wherein the electromagnetically responsive material can be responsive to the electromagnetic energy generated by the electromagnetic generator to generate the pressure waves.

In certain implementations, the electromagnetic generator can be configured to generate electromagnetic energy that causes movement of the electromagnetically responsive material in the treatment fluid to generate the pressure waves in the treatment fluid. In certain implementations, the electromagnetically responsive medium comprises a plurality of ferrous particles. In certain implementations, the plurality of ferrous particles are suspended within the treatment fluid. In certain implementations, the electromagnetic generator comprises a magnetic field generator. In certain implementations, the electromagnetic generator comprises a coiled wire. In certain implementations, the fluid supply extends through a diameter of the coiled wire. In certain implementations, the electromagnetic generator can be positioned within a flow path of the treatment fluid within the treatment fluid supply. In certain implementations, the electromagnetic generator can be isolated from a flow path of the treatment fluid within the treatment fluid supply. In certain implementations, the apparatus further comprises a controller configured to control operation of the electromagnetic generator. In certain implementations, the controller can be configured to send control signals to the electromagnetic generator, the control signals selected to cause the electromagnetic generator to generate electromagnetic energy which causes a response in the electromagnetically responsive material to produce acoustic waves in the treatment fluid having a predetermined acoustic signature. In certain implementations, the apparatus further comprises a fluid platform comprising a chamber to be positioned against the tooth, the chamber shaped to retain treatment fluid. In certain implementations, the pressure wave generator can be exposed to the chamber. In certain implementations, the apparatus further comprises a fluid passage in fluid communication with the chamber via an opening at a distal end of the fluid passage, the fluid passage configured to deliver treatment fluid to the chamber through the opening. In certain implementations, the pressure wave generator can be positioned to generate pressure waves in the treatment fluid at a location within the fluid passage proximal of the opening and outside of the tooth, the generated pressure waves being transmitted through the treatment fluid in the fluid passage and the chamber to the treatment region of the tooth. In certain implementations, the apparatus further comprises a fluid motion generator configured to generate bulk fluid motion in the treatment fluid. In certain implementations, the pressure wave generator can be configured to generate bulk fluid motion in the treatment fluid.

In yet another embodiments, an apparatus for treating a tooth is disclosed. The apparatus can comprises a treatment fluid supply configured to supply a treatment fluid to a treatment region of the tooth and pressure wave generator configured to generate pressure waves in the treatment fluid. The pressure wave generator can comprise a volume housing a electromagnetically responsive material and an electromagnetic energy generator configured to generate electromagnetic energy. The electromagnetically responsive material can be responsive to the electromagnetic energy generated by the electromagnetic generator so as to generate pressure waves in the treatment fluid.

In certain implementations, the electromagnetically responsive material comprises a ferrofluid. In certain implementations, the electromagnetic generator comprises a magnetic field generator. In certain implementations, the electromagnetic generator comprises a coiled wire. In certain implementations, the volume housing the electromagnetically responsive material can be located within a diameter of the coiled wire. In certain implementations, the treatment fluid supply extends through a diameter of the coiled wire. In certain implementations, the electromagnetic generator can be positioned within a flow path of the treatment fluid within the treatment fluid supply. In certain implementations, the electromagnetic generator can be isolated from a flow path of the treatment fluid within the treatment fluid supply. In certain implementations, the apparatus further comprises a deformable material separating the volume housing the electromagnetically responsive material and the electromagnetic generator. In certain implementations, the electromagnetically responsive material can be responsive to the electromagnetic energy generated by the electromagnetic generator so as to generate pressure waves in the treatment fluid. In certain implementations, the apparatus further comprises a controller configured to control operation of the electromagnetic generator. In certain implementations, the controller can be configured to send control signals to the electromagnetic generator, the control signals selected to cause the electromagnetic generator to generate electromagnetic energy which causes a response in the electromagnetically responsive material to produce acoustic waves in the treatment fluid having a predetermined acoustic signature. In certain implementations, the apparatus further comprises a fluid platform comprising a chamber to be positioned against the tooth, the chamber shaped to retain treatment fluid. In certain implementations, the pressure wave generator can be exposed to the chamber. In certain implementations, the apparatus further comprises a fluid passage in fluid communication with the chamber via an opening at a distal end of the fluid passage, the fluid passage configured to deliver treatment fluid to the chamber through the opening. In certain implementations, the pressure wave generator can be positioned to generate pressure waves in the treatment fluid at a location within the fluid passage proximal of the opening and outside of the tooth, the generated pressure waves being transmitted through the treatment fluid in the fluid passage and the chamber to the treatment region of the tooth. In certain implementations, the apparatus further comprises a fluid motion generator configured to generate bulk fluid motion in the treatment fluid. In certain implementations, the pressure wave generator can be configured to generate bulk fluid motion in the treatment fluid.

In yet another embodiment, an apparatus for treating a treatment region of a tooth is disclosed. The apparatus can comprise a fluid platform comprising a chamber to be positioned against the tooth, the chamber shaped to retain treatment fluid, a fluid passage in fluid communication with the chamber via an opening at a distal end of the fluid passage, the fluid passage in fluid communication with the chamber through the opening, and a pressure wave generator positioned to generate pressure waves in the treatment fluid at a location within the fluid passage proximal of the opening and outside of the tooth, the generated pressure waves being transmitted through the treatment fluid in the chamber to the treatment region of the tooth.

In certain implementations, the pressure wave generator comprises an electromagnetic generator. In certain implementations, the electromagnetic generator can be configured to convert electromagnetic energy to pressure waves in the treatment fluid by way of an electromagnetically responsive medium. In certain implementations, the apparatus further comprises a controller configured to send control signals to electromagnetic generator, the control signals selected to cause the electromagnetic generator to generate electromagnetic energy that produces acoustic waves in the treatment fluid having a predetermined acoustic signature. In certain implementations, the electromagnetic generator comprises a magnetic field generator. In certain implementations, the electromagnetically responsive medium comprises a plurality of ferrous particles. In certain implementations, the plurality of ferrous particles are suspended within the treatment fluid. In certain implementations, the electromagnetic generator can be configured to generate electromagnetic energy that causes movement of the ferrous particles in the fluid which produces waves in the fluid having the predetermined acoustic signature. In certain implementations, the apparatus further comprises a volume housing the electromagnetically responsive medium and a diaphragm comprising a first side and a second side, the first side exposed to the volume, the second side exposed to the fluid, the diaphragm being movable such that movement of the electromagnetically responsive medium within the volume causes movement of the diaphragm. In certain implementations, the apparatus further comprises a deformable material separating the volume housing the electromagnetically responsive material and the electromagnetic generator. In certain implementations, the electromagnetically responsive medium comprise a dielectric that separates two conductive plates. In certain implementations, the electromagnetically responsive medium comprises one or more magnets. In certain implementations, the pressure wave generator comprises a liquid jet apparatus. In certain implementations, the liquid jet apparatus comprises a nozzle configured to produce a high velocity liquid jet. In certain implementations, the pressure wave generator further comprises an impingement plate. In certain implementations, the pressure wave generator comprises a sonic, ultrasonic, or megasonic device. In certain implementations, the pressure wave generator comprises a mechanical stirrer. In certain implementations, the pressure wave generator comprises a laser device configured to propagate optical energy within the treatment fluid. In certain implementations, the apparatus further comprises a fluid motion generator configured to generate bulk fluid motion in the treatment fluid. In certain implementations, the fluid motion generator can be located between the pressure wave generator and the opening. In certain implementations, the fluid motion generator can be located within the chamber. In certain implementations, the pressure wave generator can be configured to generate bulk fluid motion in the treatment fluid. In certain implementations, the pressure wave generator can be positioned downstream of the chamber. In certain implementations, the fluid passage can be a fluid outlet line configured to evacuate fluid from the treatment region. In certain implementations, the apparatus further comprises a vent disposed along the outlet line, the vent being exposed to ambient air. In certain implementations, the pressure wave generator can be positioned upstream of the chamber. In certain implementations, wherein the fluid passage can be a fluid inlet line. In certain implementations, the treatment fluid comprises a degassed liquid. In certain implementations, the generated pressure waves have a broadband power spectrum and multiple frequencies.

In yet another embodiment, a method for treating a treatment region of a tooth is disclosed. The method can comprise receiving a control signal representative of a predetermined acoustic signature and in response to receiving the control signal, generating electromagnetic waves which interact with an electromagnetically responsive medium to produce acoustic waves in a treatment fluid having the predetermined acoustic signature.

In certain implementations, generating electromagnetic waves comprises generating electromagnetic waves which act on the electromagnetically responsive material to cause the electromagnetically responsive material to move within the treatment fluid. In certain implementations, the electromagnetically responsive material comprises electromagnetic particles within the treatment fluid. In certain implementations, generating electromagnetic waves comprises generating electromagnetic waves which act on the electromagnetically responsive material to cause movements of the electromagnetically responsive material which move a diaphragm in communication with the treatment fluid. In certain implementations, the electromagnetically responsive material comprises a ferrofluid. In certain implementations, generating electromagnetic waves comprises generating electromagnetic waves which interact with the electromagnetically responsive medium to produce acoustic waves in the treatment fluid within a fluid passage in fluid communication with a chamber of a fluid platform via an opening, the fluid platform positioned against the tooth, wherein the acoustic waves are produced in the treatment fluid proximal of the opening and outside of the tooth. In certain implementations, generating electromagnetic waves comprises generating a magnetic field. In certain implementations, generating a magnetic field comprises generating a changing magnetic field at one or a plurality of frequencies and/or pulsation patterns that correspond to the predetermined acoustic signature. In certain implementations, the method further comprises generating bulk fluid motion in the treatment fluid. In certain implementations, the method further comprises imaging the tooth. In certain implementations, the method further comprises determining the predetermined acoustic signature based on the imaging of the tooth. In certain implementations, the method further comprises measuring an acoustic signature of acoustic waves produced in the treatment fluid during a treatment procedure. In certain implementations, the method further comprises adjusting the control signal based on the measured acoustic signature. In certain implementations, the method further comprises comparing the measured acoustic signature to the predetermined acoustic signature, wherein adjusting the control signal based on the measured acoustic signature comprises adjusting the control signal based on the comparison between the measured acoustic signature and the predetermined acoustic signature. In certain implementations, the treatment region comprises a root canal of the tooth. In certain implementations, the method further comprises cleaning the root canals with the acoustic waves. In certain implementations, the method further comprises filling the root canal with a filling material. In certain implementations, the treatment region comprises an exterior surface of the tooth. In certain implementations, the method further comprises cleaning the exterior surface of the tooth with the acoustic waves. In certain implementations, the method further comprises filling a treated carious region on the exterior surface of the tooth.

In yet another embodiment, a method of treating a tooth is disclosed. The method can comprise positioning a fluid platform comprising a chamber at or near a treatment region of the tooth, generating pressure waves in a treatment fluid in a fluid passage disposed proximal of an opening that provides fluid communication between the fluid passage and the chamber and propagating the generated pressure waves through the fluid passage and the chamber to the treatment region to treat the tooth.

In certain implementations, generating pressure waves comprises generating electromagnetic waves which interact with an electromagnetically responsive medium to produce pressure waves in a treatment fluid. In certain implementations, generating electromagnetic waves comprises generating electromagnetic waves which act on the electromagnetically responsive material to cause the electromagnetically responsive material to move within the treatment fluid. In certain implementations, the electromagnetically responsive material comprises electromagnetic particles within the treatment fluid. In certain implementations, generating electromagnetic waves comprises generating electromagnetic waves which act on the electromagnetically responsive material to cause movements of the electromagnetically responsive material which move a diaphragm in communication with the treatment fluid. In certain implementations, the electromagnetically responsive material comprises a ferrofluid. In certain implementations, generating electromagnetic waves comprises generating a magnetic field. In certain implementations, generating a magnetic field comprises generating a changing magnetic field at one or a plurality of frequencies and/or pulsation patterns that correspond to the predetermined acoustic signature. In certain implementations, the method further comprises generating bulk fluid motion in the treatment fluid. In certain implementations, the method further comprises receiving a control signal representative of a predetermined acoustic signature, and, in response to receiving the control signal, generating the electromagnetic waves to produce acoustic waves in the treatment fluid having the predetermined acoustic signature. In certain implementations, the method further comprises imaging the tooth. In certain implementations, the method further comprises determining the predetermined acoustic signature based on the imaging of the tooth. In certain implementations, the method further comprises measuring an acoustic signature of acoustic waves produced in the treatment fluid during a treatment procedure. In certain implementations, the method further comprises adjusting the control signal based on the measured acoustic signature. In certain implementations, the method further comprises comparing the measured acoustic signature to the predetermined acoustic signature, wherein adjusting the control signal based on the measured acoustic signature comprises adjusting the control signal based on the comparison between the measured acoustic signature and the predetermined acoustic signature. In certain implementations, the treatment region comprises a root canal of the tooth. In certain implementations, the method further comprises cleaning the root canal with the acoustic waves. In certain implementations, the method further comprises filling the root canal with a filling material. In certain implementations, the treatment region comprises an exterior surface of the tooth. In certain implementations, the method further comprises cleaning the exterior surface of the tooth with the acoustic waves. In certain implementations, the method further comprises filling a treated carious region on the exterior surface of the tooth.

In another embodiment, an apparatus for treating a treatment region of a tooth is disclosed. The apparatus can include a first fluid supply configured to supply a treatment fluid to the tooth. The apparatus can include a second fluid supply configured to supply the treatment fluid to the first fluid supply, the second fluid supply positioned proximal the first fluid supply and having a volume that is different from a volume of the first fluid supply. The apparatus can include a pressure wave generator positioned to generate pressure waves in the treatment fluid at a location within the second fluid supply, the generated pressure waves being transmitted through the treatment fluid in the first fluid supply to the treatment region of the tooth.

In some embodiments, the first fluid supply comprises a fluid platform including a chamber to be positioned against the tooth and the second fluid supply comprises a fluid supply line in fluid communication with the chamber. In some embodiments, an opening is disposed between the first and second fluid supplies that provides fluid communication therebetween. In some embodiments, the pressure wave generator comprises an electromagnetic generator configured to act upon an electromagnetically responsive material to generate the pressure waves in the treatment fluid.

For purposes of this summary, certain aspects, advantages, and novel features of certain disclosed inventions are summarized. It is to be understood that not necessarily all such advantages may be achieved in accordance with any particular embodiment of the invention. Thus, for example, those skilled in the art will recognize that the inventions disclosed herein may be embodied or carried out in a manner that achieves one advantage or group of advantages as taught herein without necessarily achieving other advantages as may be taught or suggested herein. Further, the foregoing is intended to summarize certain disclosed inventions and is not intended to limit the scope of the inventions disclosed herein.

### BRIEF DESCRIPTION OF THE DRAWINGS

FIG. 1 is a cross-section view schematically illustrating a root canal system of a tooth.

FIG. 2 schematically illustrates an example of a system for treating a tooth with a pressure wave generator and a fluid motion generator.

FIG. 3 schematically illustrates an example of a system for treating a tooth with a pressure wave generator and a fluid motion generator.

FIG. 4 schematically illustrates an example of a treatment instrument with a pressure wave generator.

FIG. 5 is a cross-section view schematically illustrating an example of a treatment instrument with a pressure wave generator and a volume with an electromagnetically responsive material.

FIG. 6 is a cross-section view schematically illustrating an example of a treatment instrument with a pressure wave generator and a volume with an electromagnetically responsive material.

FIG. 7 is a cross-section view schematically illustrating an example of a treatment instrument with a pressure wave generator and a volume with an electromagnetically responsive material.

FIG. 8 is a cross-section view schematically illustrating an example of a treatment instrument with a pressure wave generator and a volume with an electromagnetically responsive material positioned over a treatment region.

FIG. 9 is a cross-section view schematically illustrating an example of a treatment instrument with a pressure wave generator and a volume with an electromagnetically responsive material.

FIG. 10 is a cross-section view schematically illustrating an example of a treatment instrument with a pressure wave generator and an electromagnetically responsive material suspended in a treatment fluid.

FIG. 11 is a cross-section view schematically illustrating an example of a treatment instrument with a pressure wave generator and an electromagnetically responsive material suspended in a treatment fluid.

FIG. 12 is a cross-section view schematically illustrating an example of a treatment instrument with a pressure wave generator and an electromagnetically responsive material suspended in a treatment fluid.

FIG. 13 is a cross-section view schematically illustrating an example of a treatment instrument with an electromagnetic generator and a volume located in a treatment region.

FIG. 14 is a cross-section view schematically illustrating an example of treatment instrument and a fluid platform.

FIG. 15 schematically illustrates an example of a fluid motion generator.

FIG. 16 is a cross-section view schematically illustrating an example of a fluid motion generator.

FIG. 17 is a cross-section view schematically illustrating an example of a fluid motion generator.

FIG. 18A schematically illustrates an example of acoustic power generated by a liquid jet.

FIG. 18B schematically illustrates an example of acoustic power generated by an ultrasonic transducer.

FIG. 18C schematically illustrates an example graph of an acoustic power spectrum.

FIG. 19 is a flowchart illustrating an example method for treating a treatment region of a tooth.

FIG. 20 is a flowchart illustrating an example method for treating a treatment region of a tooth.

Throughout the drawings, reference numbers may be re-used to indicate a general correspondence between referenced elements. The drawings are provided to illustrate example embodiments described herein and are not intended to limit the scope of the disclosure.

### DETAILED DESCRIPTION

Various embodiments of apparatuses for treating at least a treatment region of a tooth are disclosed in detail hereinafter. Examples of methods for treating a treatment region of a tooth are disclosed in detail.

Various embodiments disclosed herein utilize a pressure wave generator to treat a treatment region of a tooth, e.g., to clean or obturate a root canal, to clean or fill a carious region on an exterior surface of the tooth, to remove dental deposits from the tooth or gums, to bleach or whiten the tooth, etc.

Various embodiments disclosed herein utilize pressure wave generators to convert electromagnetic energy to acoustic waves in a fluid by way of an electromagnetically responsive medium. For example, as described herein, in magnetic applications, an electromagnetically responsive medium may include a ferrofluid or a plurality of ferrous particles. An electromagnetic generator can be activated to induce motion of the electromagnetically responsive medium. Motion of the electromagnetically responsive medium can, in some examples, induce vibrations and acoustic waves in a treatment fluid for instance used to clean or fill the treatment region. Additionally, or alternatively, in capacitive or electrostatic applications, an electromagnetically responsive medium may include a dielectric that preferably separates two grids or plates. In some embodiments, a modulated multifrequency control can be provided in capacitive or electrostatic applications in order to create acoustic waves. In other applications, an electromagnetically responsive medium may additionally or alternatively comprise a mechanical device or a portion of a mechanical device, such as, for example, a vibrating magnetic component.

Various embodiments disclosed herein describe devices, systems, and methods for filling a treatment region of a tooth, including, e.g., obturation of a treated root canal and filling or restoration of a treated carious region. Obturation can include holding and delivering flowable material into a range of molar, anterior, or pre-molar root canal systems to seal entries into the root canal systems. Upon delivery, the flowable material within the root canal system may be cured in various embodiments, e.g., cured by heating, exposure to light, and/or resting without application of energy to the tooth. Similarly, in various embodiments, a flowable filling or restorative material may be flowed into and/or onto the treated carious region to fill the treated region. In some embodiments, the filling or restorative region may be cured in any suitable manner.

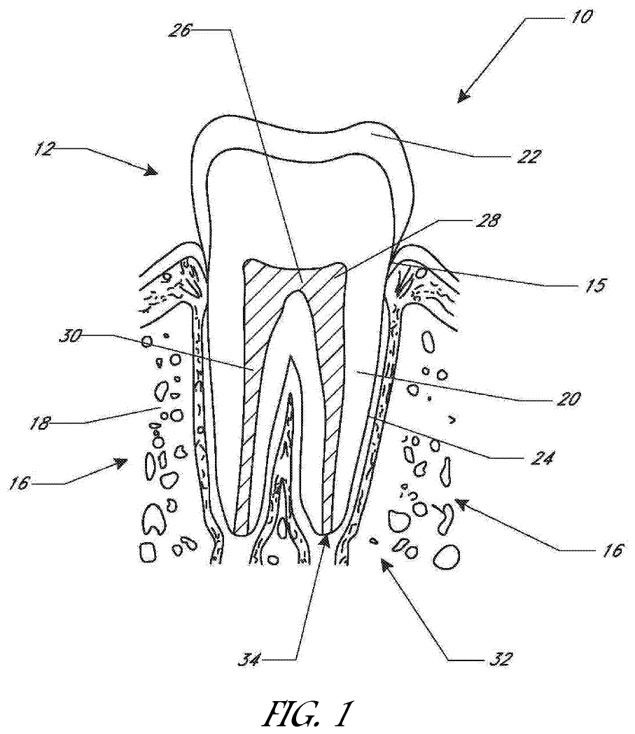

FIG. 1 is a cross section schematically illustrating a typical human tooth 10, which comprises a crown 12 extending above the gum tissue 14 and at least one root 16 set into a socket (alveolus) within the jaw bone 18. Although the tooth 10 schematically depicted in FIG. 1 is a molar, the apparatus and methods described herein may be used on any type of tooth such as an incisor, a canine, a bicuspid, or a molar. The hard tissue of the tooth 10 includes dentin 20 which provides the primary structure of the tooth 10, a very hard enamel layer 22 which covers the crown 12 to a cementoenamel junction 15 near the gum 14, and cementum 24 which covers the dentin 20 of the tooth 10 below the cementoenamel junction 15.

A pulp cavity 26 is defined within the dentin 20. The pulp cavity 26 comprises a pulp chamber 28 in the crown 11 and a root canal space 30 extending toward an apex 32 of each root 16. The pulp cavity 26 contains dental pulp, which is a soft, vascular tissue comprising nerves, blood vessels, connective tissue, odontoblasts, and other tissue and cellular components. The pulp provides innervation and sustenance to the tooth through the epithelial lining of the pulp chamber 26 and the root canal space 30. Blood vessels and nerves enter/exit the root canal space 30 through a tiny opening, the apical foramen 32, near a tip of the apex 32 of the root 16.

Various embodiments disclosed herein can effectively and safely remove unhealthy material from a treatment region of a tooth, e.g., from within the tooth and/or from outside surfaces of the tooth. For example, the embodiments described herein can provide improved control over the acoustic properties (e.g., frequency or frequencies) of pressure waves generated for removal of unhealthy material or for other dental treatment procedures. In particular, the embodiments disclosed herein can remove unhealthy materials, such as unhealthy organic matter, inorganic matter, pulp tissue, caries, stains, calculus, plaque, biofilm, bacteria, pus, decayed tooth matter, and food remnants from the treatment region without substantially damaging healthy dentin or enamel. For example, the disclosed apparatus, methods, and compositions advantageously may be used with root canal cleaning treatments, e.g., to efficiently remove unhealthy or undesirable materials such as organic and/or inorganic matter from a root canal system and/or to disinfect the root canal system. Organic material (or organic matter) includes organic substances typically found in healthy or diseased teeth or root canal systems such as, for example, soft tissue, pulp, blood vessels, nerves, connective tissue, cellular matter, pus, and microorganisms, whether living, inflamed, infected, diseased, necrotic, or decomposed. Inorganic matter includes calcified tissue and calcified structures, which are frequently present in the root canal system. In some embodiments, the root canal can be filled with an obturation material (e.g., a flowable obturation material that can be hardened into a solid or semi-solid state, gutta percha or other solid or semi-solid materials) after treatment of the root canal. The embodiments described herein can provide improved control over the acoustic properties (e.g., frequency or frequencies) of pressure waves generated for filling a treatment region with obturation materials.

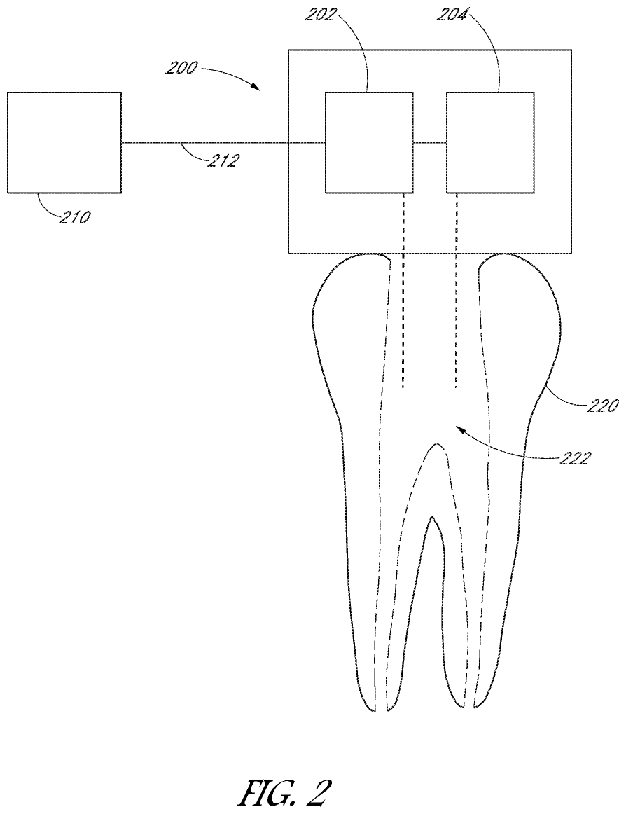

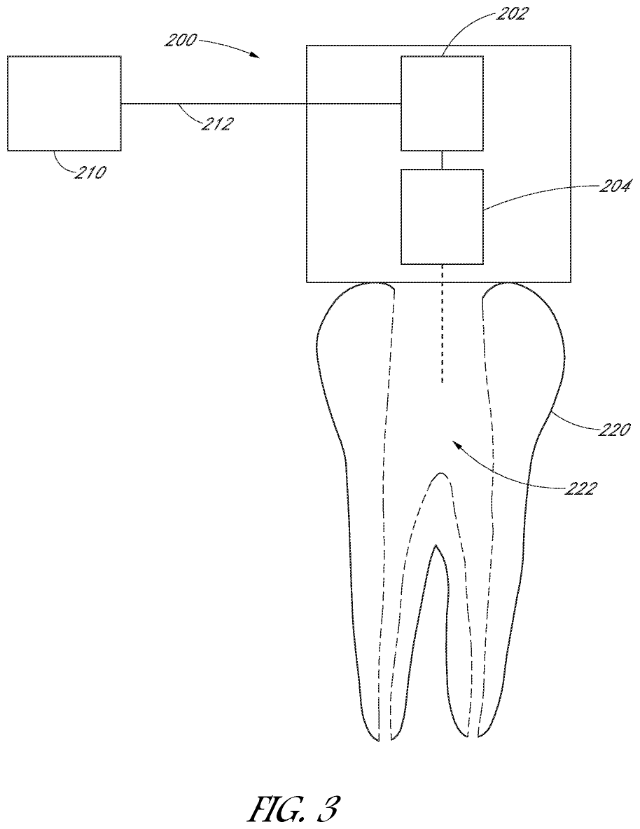

FIGS. 2 and 3 are schematic diagrams of a treatment instrument or apparatus comprising a handpiece 200 housing a pressure wave generator 202. In some embodiments, as shown in FIGS. 2 and 3, the handpiece 200 can additionally or alternatively house a fluid motion generator 204. As described in further detail herein, the pressure wave generator 202 can be configured to generate pressure waves in a treatment fluid during a dental treatment procedure. The generated pressure waves can have sufficient energy so as to propagate throughout the treatment region within the treatment fluid to clean or fill the treatment region. In various embodiments, as described herein, the generated pressure waves can comprise a broadband power spectrum. As described in further detail herein, the fluid motion generator 204 can be configured to generate bulk fluid motion within the treatment fluid during a dental treatment procedure. The generated bulk fluid motion can serve to irrigate diseased tissue from the tooth and/or to assist in circulating filling material throughout the treatment region.

As shown in FIG. 2, the pressure wave generator 202 and fluid motion generator 204 can be, in examples in use during a treatment procedure, in fluid communication with a treatment region 222 of the tooth 220. The treatment region shown in FIG. 2 is a root canal of the tooth; in other embodiments, the treatment region can comprise an exterior surface of the tooth and/or surrounding gum tissue. For example, in some embodiments, the pressure wave generator 202 and the fluid motion generator 204 can be disposed within or in fluid communication with a chamber of a fluid platform positioned against the tooth in use of the treatment apparatus. The pressure wave generator 202 can be configured to generate pressure waves within the treatment fluid in the chamber so that the pressure waves propagate through the treatment fluid in the chamber to the treatment region 222 of the tooth 220. Additionally or alternatively, the fluid motion generator 204 can be configured to generate bulk fluid motion within the treatment fluid in the chamber so that the bulk fluid motion propagates through the treatment fluid in the chamber to the treatment region 222 of the tooth 220.

In some embodiments, the pressure wave generator 202 and fluid motion generator 204 can be disposed proximally of a chamber positioned against the tooth. For example, in some embodiments, the pressure wave generator 202 and fluid motion generator 204 can be positioned in one or more fluid passages in fluid communication with the chamber via an opening at a distal end of the fluid passage. In some embodiments, the pressure wave generator 202 can be positioned to generate pressure waves in the treatment fluid at a location within a fluid passage proximal of the opening and outside of the tooth 220. In some embodiments, additionally or alternatively, the fluid motion generator 204 can be positioned to generate bulk fluid motion in the treatment fluid at allocation within a fluid passage proximal of the opening and outside of the tooth 220. As described herein, a fluid passage or fluid supply may include any fluid chambers, fluid lines, fluid conduits, fluid volumes, or other suitable structures.

In some embodiments, the one or more fluid passages can be upstream of the chamber. For example, in some embodiments, a fluid passage can be a fluid inlet configured to deliver treatment fluid to the chamber and/or treatment region. In some embodiments, the one or more fluid passages can be downstream of the chamber. For example, a fluid passage can be a fluid outlet configured to evacuate fluid from the chamber and/or treatment region. In some embodiments, the fluid passage can comprise a reservoir or other volume that is disposed proximal the distal end of the treatment instrument, e.g., proximal of the opening to the reservoir or other volume. In some embodiments, one of the pressure wave generator 202 and fluid motion generator 204 can be positioned within a fluid passage upstream of the chamber and the other of the pressure wave generator 202 and fluid motion generator 204 can be positioned in a fluid passage downstream of the volume.

As shown in FIG. 2, the pressure wave generator 202 and fluid motion generator 204 are positioned in a parallel arrangement. As shown schematically by the dashed lines extending from the pressure wave generator 202 and the fluid motion generator 204 to the treatment region 222, the pressure wave generator 202 and the fluid motion generator 204 can separately fluidly communicate with the treatment region 222 of the tooth. The pressure wave generator 202 and fluid motion generator 204 can be positioned so that the pressure wave generator 202 and fluid motion generator 204 generate pressure waves and bulk fluid motion, respectively, within the treatment. In the illustrated parallel arrangement, the pressure waves and fluid motion may be generated separately or independently from one another. For example, the pressure wave generator II and the fluid motion generator FMG may comprise different devices that respectively generate pressure waves and fluid motion. In some embodiments, both of the pressure wave generator 202 and the fluid motion generator 204 can be positioned within the chamber of the handpiece 200 so as to generate pressure waves and bulk fluid motion within the treatment fluid simultaneously. Alternatively, in some embodiments, the pressure wave generator 202 can be positioned within a first fluid passage and the fluid motion generator 204 can be positioned within a second fluid passage that open to the chamber so that the pressure wave generator 202 generates pressure waves that propagate through the first passage into the chamber and the fluid motion generator 204 generates bulk fluid motion that propagates through the second passage into the chamber.

In other embodiments, as shown in FIG. 3, the pressure wave generator 202 and fluid motion generator 204 can be positioned in a series arrangement. In such embodiments, the pressure wave generator 202 can be positioned to generate pressure waves upstream of the location at which the fluid motion generator 204 generates bulk fluid motion within the flow path of the treatment fluid, or the pressure wave generator 202 can be positioned to generate pressure waves downstream of the location at which the fluid motion generator 204 generates bulk fluid motion. For example, in some embodiments, both of the pressure wave generator 202 and fluid motion generator 204 can be positioned in the same fluid passage and one of the pressure wave generator 202 and the fluid motion generator 204 can be positioned upstream of the other of the pressure wave generator 202 and the fluid motion generator 204 within the fluid passage. In other embodiments, one of the pressure wave generator 202 and the fluid motion generator 204 can be positioned in a fluid passage and the other of the pressure wave generator 202 and the fluid motion generator 204 can be positioned within the chamber. In some embodiments, the pressure wave generator 202 and the fluid motion generator 204 are separate devices or systems, as described in embodiments herein. Alternatively or additionally, in some embodiments, the pressure wave generator 202 and the fluid motion generator 204 may be the same device.

As shown in FIGS. 2 and 3, in some embodiments, the handpiece 200 can be coupled to a controller 210. One or both of the pressure wave generator 202 and fluid motion generator 204 can be electrically connected to a controller 210 via one or more electrical communication channels, e.g., a wire 212. In some embodiments, the controller 210 can send control signals to the one or both of the pressure wave generator 202 and fluid motion generator 204. In some embodiments, the control signals can be selected to cause the pressure wave generator to generate pressure waves. In some embodiments, the control signals can be selected to cause the pressure wave generator to generate pressure waves having a predetermined acoustic signature. Additionally or alternatively, in some embodiments, the control signals can be selected to cause the fluid motion generator to generate bulk fluid motion.

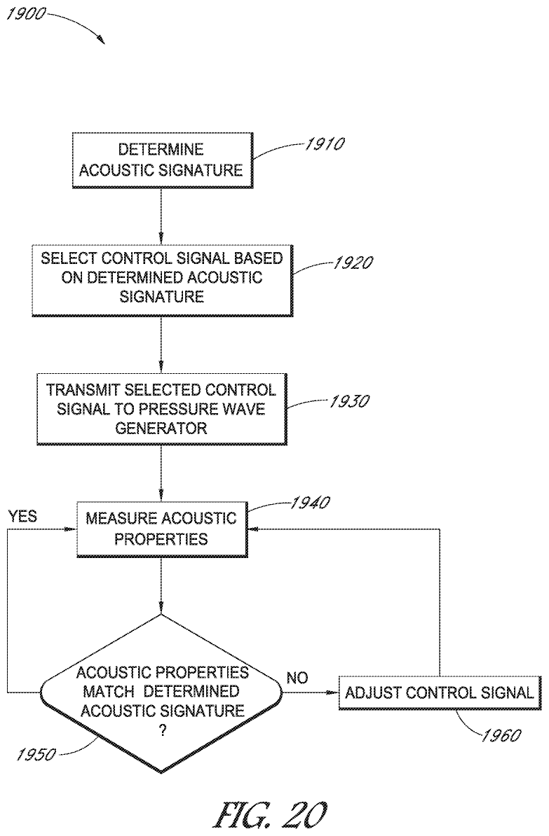

In some embodiments, an acoustic signature may refer to a set of acoustic properties of pressure waves generated in a treatment region, such as for example, a set of relative or absolute acoustic power levels at a corresponding acoustic frequency or band of acoustic frequencies, a representative bandwidth of the acoustic profile to be delivered to the tooth, etc. In some embodiments, a predetermined acoustic signature can comprise an acoustic signature stored in a memory device determined prior to a dental treatment procedure (for example, by imaging). Additionally or alternatively, in some embodiments, a predetermined acoustic signature can be calculated based on various input parameters that are used and updated during a dental treatment procedure. Further examples of determining a predetermined acoustic frequency are described with respect to FIGS. 19 and 20. The acoustic signature can be determined in a patient-specific manner by calculating or otherwise determining the acoustic signature that is sufficient to treat the tooth (e.g., sufficient to clean or fill the treatment region).

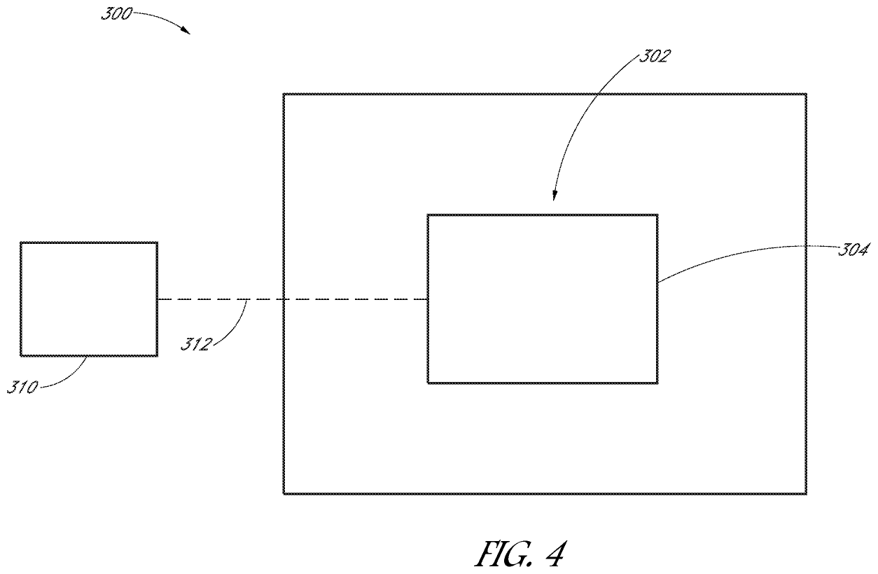

FIG. 4 is a schematic diagram of a treatment instrument or apparatus comprising a handpiece 300 housing a pressure wave generator 302. In some embodiments, the handpiece 300 can be positioned against the treatment region of the tooth in use. In other embodiments, however, the treatment instrument can comprise a cap that can be attached or otherwise coupled to the tooth. As shown in FIG. 4, the pressure wave generator 302 can include an electromagnetic generator 304. The electromagnetic generator 304 can be configured to generate electromagnetic energy. In certain embodiments, the electromagnetic energy generated by the electromagnetic generator 304 can interact with an electromagnetically responsive material. For example, in some embodiments, the electromagnetic generator 304 can be programmed, positioned, and/or otherwise configured to cause movement of an electromagnetically responsive material.

In some embodiments, the electromagnetic generator 304 can generate electromagnetic energy so as to cause movement of an electromagnetically responsive material that results in the formation of pressure waves in a treatment fluid (for example, a cleaning treatment fluid or a filling treatment fluid such as an obturation material). For example, in some embodiments, a treatment fluid can include an electromagnetically responsive material. In such embodiments, the electromagnetic generator 304 can generate electromagnetic energy that causes the electromagnetically responsive material to move within the treatment fluid so as to generate pressure waves therein.

In some embodiments, the electromagnetic generator 304 can generate electromagnetic energy that causes the electromagnetically responsive particles within the treatment fluid to move at a specific frequency or bands of multiple frequencies. As the electromagnetically responsive material moves, the treatment fluid can move at the specific frequency or a corresponding frequency (or at multiple frequencies), which creates pressure waves in the treatment fluid. In some embodiments, as explained herein, the generated pressure waves have a broadband power spectrum. In some embodiments, the handpiece 300 of FIG. 4 can be used to generate pressure waves throughout a treatment region of a tooth. In some embodiments, the pressure waves generated in the treatment fluid can propagate throughout the treatment region to treat the tooth once the treatment fluid is delivered to the treatment region.

The electromagnetic generator 304 can be positioned within the handpiece 300 at any suitable location. In some embodiments, the electromagnetic generator 304 can be positioned at any suitable location within the handpiece 300 sufficient for generated electromagnetic energy to cause movement of an electromagnetically responsive material in a treatment fluid within a treatment region of the tooth. For example, in some embodiments, the electromagnetic generator 304 can be installed at or adjacent an end or other exterior surface of the handpiece 300, allowing the handpiece 300 and electromagnetic generator 304 within to be positioned over or adjacent a treatment fluid containing electromagnetically responsive material.

Additionally or alternatively, in some embodiments, the electromagnetic generator 304 can be positioned at any suitable location within the handpiece 300 sufficient for generated electromagnetic energy to cause movement of an electromagnetically responsive material positioned within a portion of the handpiece 300. For example, the electromagnetic generator 304 can be installed around a fluid port or channel that carries treatment fluid having an electromagnetically responsive material.

In some embodiments, the handpiece 300 can be coupled to a controller 310. The electromagnetic generator 304 can be electrically connected to controller 310 via a wire 312. In some embodiments, the controller 310 can send control signals to the electromagnetic generator 304. The control signals can be selected to cause the electromagnetic generator 304 to produce electromagnetic energy. The generated electromagnetic energy can cause the electromagnetically responsive material to move at one or more frequencies that corresponds to instructions in the control signal sent to the electromagnetic generator 304. Movement of the electromagnetically responsive material can result in the production of acoustic waves in a fluid, such as a treatment fluid, having a determined acoustic signature.

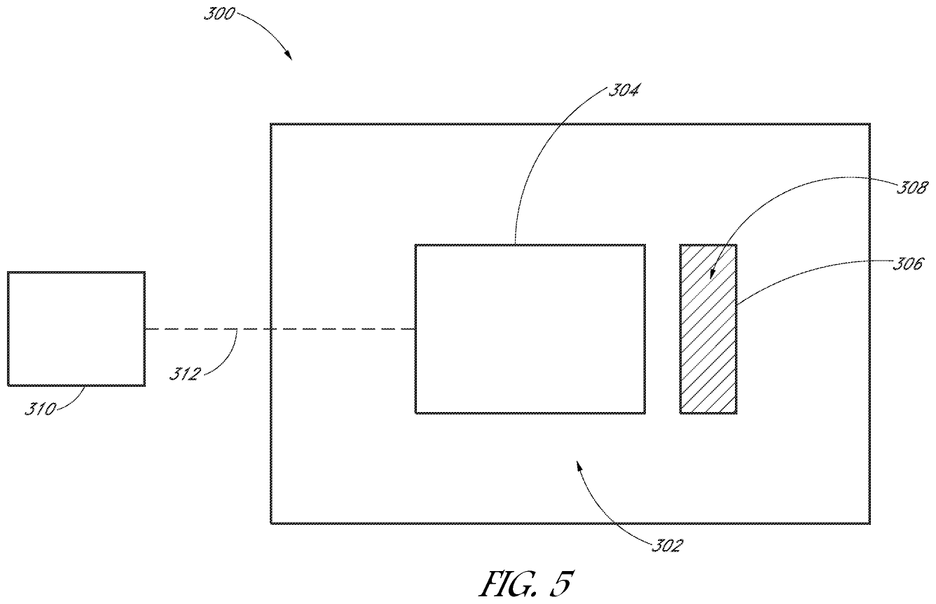

Additionally or alternatively, in some embodiments, an electromagnetically responsive material can be positioned within a volume, such as a reservoir or membrane, separate from the treatment fluid. FIG. 5 is a schematic diagram of another embodiment of the handpiece 300 including a volume 306, and an electromagnetically responsive material 308. In some embodiments, the volume 306 can be a reservoir, chamber, membrane, or any other suitable structure.

The electromagnetically responsive material 308 can be encapsulated within the volume 306. The volume 306 can be positioned at any suitable location within the handpiece 300 and/or at any suitable location relative to the electromagnetic generator 304. For example, in some embodiments, the volume 306 can be coupled to or can abut the electromagnetic generator 304. Alternatively, in some embodiments, the volume 306 can be spaced apart from the electromagnetic generator 304. In some embodiments, the volume 306 can be positioned within a diameter of the electromagnetic generator 304. Alternatively, in other embodiments, the volume 306 can be positioned outside the diameter of the electromagnetic generator 304.

The electromagnetically responsive material 308 within the volume 306 can be responsive to electromagnetic energy generated by the electromagnetic generator 304. In some embodiments, the electromagnetically responsive material 308 can move at a frequency that corresponds to the generated electromagnetic energy. The electromagnetically responsive material 308 can comprise an incompressible liquid in some embodiments, such that the liquid can move in response to the generated electromagnetic energy. In some embodiments, the electromagnetically responsive material 308 can be flexible.

In some embodiments, movement of the electromagnetically responsive material 308 within the volume 306 can cause resulting movement in a treatment fluid positioned adjacent to or in contact with a portion of the volume 306. For example, in some embodiments, movement of the electromagnetically responsive material can cause movement of the volume 306 itself. As an example, in some embodiments, the volume 306 can be connected to the handpiece 300 (or fluid passage within the handpiece) by a flexible member, tether, or spring. Electromagnetic waves can cause the electromagnetically responsive material (e.g., a ferrofluid or other responsive material) to move or vibrate (for example, within the handpiece in some examples), which can in turn create acoustic waves in the treatment fluid. Additionally or alternatively, in some embodiments, as described in further detail herein, a diaphragm can be coupled to, exposed to, and/or forms part of the volume 306. In some embodiments, movement of the electromagnetically responsive material within the volume 306 can cause movement of the volume 306 and/or diaphragm. For example, in some embodiments, the volume 306 can be flexible and at least a portion of the volume 306 (e.g., the diaphragm) can expand or flex so as to impart vibrations to the treatment fluid to create acoustic waves. For example, in some embodiments, the electromagnetically responsive material can comprise an incompressible fluid within the volume. In some examples, a gas (such as air) or other compressible material can also be provided in the volume such that the incompressible electromagnetically responsive material can cause the volume 306 to expand or flex. Still other configurations may be suitable. The diaphragm can also contact a separate component or fluid (e.g. treatment fluid) outside of the volume 306 such that movement of the diaphragm causes movement of the separate component or fluid. In some embodiments, the separate component coupled to the diaphragm can be exposed to a treatment fluid to cause movement of the treatment fluid in response to movement of the diaphragm.

In some embodiments, a diaphragm can include a thin, flexible, and low mass material. In some embodiments, the diaphragm can be installed within an opening of the volume 306 and/or form a portion of the volume 306, such as a side of the volume 306, such that the diaphragm can contact the electromagnetically responsive material 308 within the volume 306.

In some embodiments, the electromagnetic generator 304 can generate electromagnetic energy that causes the electromagnetically responsive material 308 to move at a specific fluid frequency (or multiple fluid frequencies). As the electromagnetically responsive material 308 moves, the electromagnetically responsive material 308 can vibrate the diaphragm, for example, at the specific frequency or a corresponding frequency (or multiple frequencies). Vibration of the diaphragm can generate pressure waves in a treatment fluid exposed to the diaphragm at the specific frequency or a corresponding frequency (or multiple frequencies). As explained herein, the generated pressure waves can have a broadband power spectrum and multiple frequencies. In some embodiments, the handpiece 300 of FIG. 5 can be used to generate pressure waves throughout a treatment region of a tooth to clean or fill the treatment region. In some embodiments, the pressure waves generated in the treatment fluid can propagate throughout the treatment region to treat the tooth once the treatment fluid is delivered to the treatment region. In various embodiments, the electromagnetic generator can produce pressure waves in the treatment fluid having a broadband power spectrum.

In some embodiments, in addition to the electromagnetically responsive material 308, the volume can include any mixture of different carrier fluids to provide a viscosity suitable for achieving a desired frequency or bandwidth using the pressure wave generator 302 of FIG. 5. For example, in some embodiments, a mixture of different carrier fluids can be included in the volume to provide a viscosity that provides for damping. Additionally or alternatively, in some embodiments, the electromagnetically responsive material 308 can include a plurality of different ferromagnetic particles having different properties that provide different frequency responses. Additionally or alternatively, in some embodiments, the electromagnetically responsive material 308 can include a plurality of fluids having different ferromagnetic particles having different properties that provide different frequency responses. In some embodiments, additional fluids having different ferromagnetic particles having different properties can be added to the electromagnetically responsive material 308 within the volume 306.

### Electromagnetic Generators

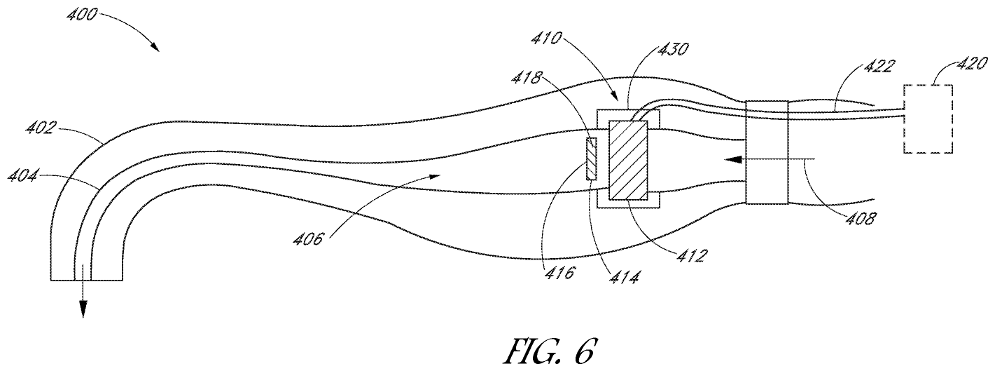

Examples of electromagnetic generators 304 and 412 are described herein with respect to FIGS. 3-14. The electromagnetic generators 304 and 412 may have any of the same or similar features or functions. Further details of the electromagnetic generators 412 and associated components are discussed with reference to FIGS. 6-14 below.

In various embodiments, the electromagnetic generator 304 or 412 can be configured to generate a magnetic field. In some embodiments, the electromagnetic generator 304 or 412 can include a magnetic field generator. In some embodiments, a magnetic field can be generated by inducing an alternating electric current on a wire conductor. The magnetic field generated by the magnetic field generator can cause movement of a magnetically responsive material, such as, for example, a ferrofluid and/or ferromagnetic particles. In such embodiments, the electromagnetic generator 304 or 412 can generate a magnetic field so as to cause movement of a ferrofluid and/or ferromagnetic particles within a volume 306 or 414 or within a treatment fluid so as to generate pressure waves therein.

In some embodiments, the electromagnetic generator 304 or 412 described herein can include an electromagnet. In some embodiments, the electromagnetic generator 304 or 412 can include a conductor. In some embodiments, current can be generated in the conductor, for example, by a controller 310 or 420, to create a corresponding changing magnetic field at one or a plurality of frequencies and/or pulsation patterns that can correspond to a desired acoustic signature. For example, in some embodiments, the magnetic field patterns can be generated across a broad band of frequencies and at a plurality of frequencies.

In some embodiments, the electromagnetic generator 304 or 412 can include a wire or a series of wires (e.g. 2, 3, 4, 5, 6, 7, 8, 9, 10 or more wires) arranged in a coil. In some embodiments, the wires can be wrapped around a core, such as a ferromagnetic core. The wires and the core can be placed in a casing, with the casing installed within a handpiece 300 or 400. In some embodiments, the wires and the core are not placed within a casing, leaving the wires and core partially or fully exposed as it is positioned within the handpiece 300 or 400.

In various embodiments, the electromagnetic generator 304 or 412 can have a hollow center, which allows the electromagnetic generator 304 or 412 to be installed around a component of the handpiece 300 or 400. For example, in FIG. 6, an electromagnetic generator 412 is installed around a fluid flow supply 404.

In certain embodiments, the electromagnetic generator 304 or 412 can create a magnetic field when a current runs through the wiring. The magnetic field can be adjusted by changing the current or voltage supplied to the electromagnetic generator 304 or 412. Changing the current or voltage supplied to the magnetic field generator can change the strength and the direction of the magnetic field.

In some embodiments, the electromagnetic generator can interact with the electromagnetically responsive material in other ways. For example, additionally or alternatively, the electromagnetic generator can comprise a capacitive device in which a pair of spaced apart charged grids or plates, such as for example, stator plates, can be modulated to generate acoustic waves in the treatment fluid, for example, by a diaphragm or other material in fluid communication with the treatment fluid. In some embodiments, additionally or alternatively, a diaphragm can be positioned between the pair of grids and a constant charge high voltage can be applied across the grids. A multifrequency signal can be modulated on top of the high voltage to cause pressure waves to propagate from both of the grids. In some embodiments, an amplitude of the generated pressure waves can be increased by using a diaphragm having a larger surface area.

Additionally or alternatively, in some embodiments, the electromagnetic generator can comprise a magnetic actuator configured to impart electromagnetic energy to one or more magnets that can move in response to a changing electromagnetic field. Movement of the magnets can generate acoustic waves in the treatment fluid, for example, by a diaphragm or other material in fluid communication with the treatment fluid.

Additionally or alternatively, in some embodiments, an electromagnetic generator can comprise a submersible acoustic membrane device comprising a magnet, such as a Neodymium magnet, used with a flexible diaphragm for the generation of acoustic waves. In some embodiments, a coil can be suspended in a gap between poles of the magnet, and an alternating electrical multifrequency signal can be applied to the coil so that the coil moves back and forth due to Faraday's law of induction. The movement of the coil can cause movement of an attached diaphragm to produces acoustic waves.

Additionally or alternatively, in some embodiments, the electromagnetic generator can comprise one or more piezoelectric devices. For example, in some embodiments, the electromagnetic generator can comprise one or more piezo crystal oscillators. The piezo crystal oscillators can include identical or mixed frequency components coupled directly or indirectly through a sonic horn to a diaphragm. The piezoelectric devices can change size and shape in response to an applied voltage. In some embodiments, an AC voltage can be applied to the piezoelectric devices to cause changes in size and shape that cause movement of the diaphragm to produce acoustic waves. In some embodiments, each piezoelectric device can provide a single resonant frequency. In some embodiments, multiple piezoelectric devices can be implemented to provide multiple acoustic frequency.

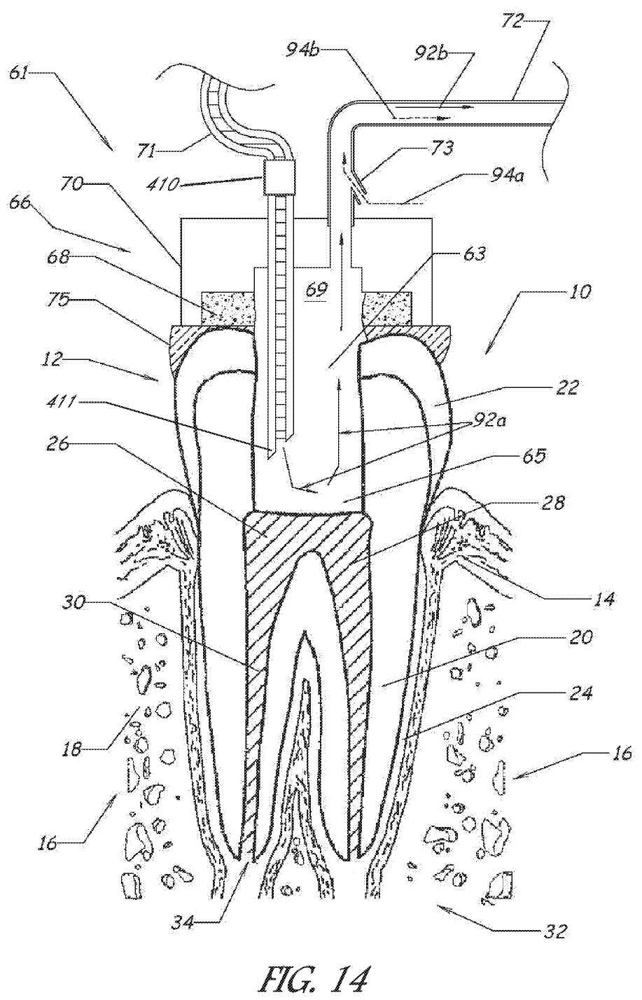

The above-described electromagnetic generators can be positioned in any suitable location within a treatment device, such as a handpiece, or treatment region of a tooth. For example, in some embodiments, an electromagnetic generator can be positioned upstream of and/or proximal to an inlet to a treatment region. In some embodiments, an electromagnetic generator can be positioned downstream of an outlet to a treatment region. In some embodiments, an electromagnetic generator can be positioned within a treatment region. In some embodiments, as shown in FIG. 14, a fluid platform can be provided at a distal end of a handpiece. The fluid platform can include a chamber configured to retain treatment fluid in the tooth. In some embodiments, the electromagnetic generator can be positioned proximal to an opening between a fluid passage, such as a fluid inlet or fluid outlet, and the chamber. In some embodiments, the electromagnetic generator can be positioned within the chamber.

### Electromagnetically Responsive Material

Examples of electromagnetically responsive materials 308 and 418 are described herein with respect to FIGS. 3-14. The electromagnetic responsive materials 308 and 418 may have any of the same or similar features or functions. Further details of the responsive materials 418 and associated components are discussed with reference to FIGS. 6-14 below.

In some embodiments, the electromagnetically responsive material can include one or more materials responsive to a magnetic field. For example, in some embodiments, the electromagnetically responsive material can include a ferrofluid. In some embodiments, the electromagnetically responsive material 308 or 418 can include ferromagnetic or ferrous particles suspended in a fluid. The ferromagnetic particles can be particles that are responsive to magnetic fields, which can include, for example, cobalt, nickel and iron. These particles can be suspended in any suitable carrier fluid. As described with respect to FIG. 4, in some embodiments, the particles can be suspended in a treatment fluid. As described with respect to FIG. 5 In some embodiments, the particles can be encapsulated within a volume 306 or 414. The volume 306 or 414 can separate the particles from the treatment fluid.

In various embodiments, the particles within the electromagnetically responsive material 308 or 418 can be sized and/or otherwise configured to avoid clumping together. In some embodiments, the particles are coated with a surfactant to avoid clumping. In response to a magnetic field, the magnetic particles within the electromagnetically responsive material 308 or 418 can move to align themselves with the magnetic field lines. Accordingly, a user can use a magnetic field to move the ferrofluid within the volume 306 or 414 or within a treatment fluid, such as treatment fluid 406.

Additionally or alternatively, in some embodiments, the electromagnetically responsive material 308 or 418 can include magnetorheological fluids (MR), which may be mixed with a carrier fluid. In some embodiments, magnetorheological fluids can mixed with or suspended in a treatment fluid. In some embodiments, magnetorheological fluids can be encapsulated within the volume 306 or 414.

Additionally or alternatively, the electromagnetically responsive material 308 or 418 can include other types of materials. For example, additionally or alternatively, in some embodiments, the electromagnetically responsive material 308 or 418 can comprise a dielectric material or a diaphragm that is disposed between two conductive grids or plates. In such embodiments, the conductive plates and intervening dielectric or diaphragm can serve as a capacitive element that moves in response to a changing charge status. Additionally or alternatively, in some embodiments, the electromagnetically responsive material 418 can comprise one or more magnets that move in response to a changing electromagnetic field. Additionally or alternatively, in some embodiments, the electromagnetically responsive material can include a coil suspended in a gap between the poles of a magnet, which can move back and forth when an alternating electrical multifrequency signal is applied. Additionally or alternatively, in some embodiments, the electromagnetically responsive material can be a piezoelectric device, which can change in size and shape when a voltage is applied. Other types of electromagnetically responsive materials may be suitable.

### Diaphragm

A diaphragm 416 is described with respect to FIGS. 6-9 below. In certain embodiments, a diaphragm 416 may also be employed in the pressure wave generator 302, for example, in connection with the volume 306.

The diaphragm 416 can comprise any thin flexible material, such as a polymer, Additionally or alternatively, in some embodiments, the diaphragm 416 can be formed of any suitable non-toxic bleach tolerant material. In some embodiments, the diaphragm 416 can be a titanium Teflon coated diaphragm. In some embodiments, a titanium, Teflon coated diaphragm 416 may be beneficial if the diaphragm 416 is positioned to contact treatment fluids during a dental treatment procedure. The thin and flexible material of the diaphragm 416 allows the diaphragm to transfer vibrations and other acoustics between two components, or between a component and a fluid (e.g. treatment fluid 406, air), or between two fluids (e.g., a ferrofluid and a treatment fluid). Accordingly, the diaphragm 416 allows two components, a component and a fluid, or two fluids to be in communication with each other. The diaphragm 416 can transfer vibrations and other acoustics between an electromagnetically responsive material 418 and a treatment fluid.

### Controller

Examples of controllers 210, 310, and 420 are described herein with respect to FIGS. 3-13. The controllers 210, 310, and 420 may have any of the same or similar features or functions. Further details of the controller 420 and associated components are discussed with reference to FIGS. 6-13 below.

As described herein, in some embodiments, the controller 210, 310, or 420 can send control signals to the electromagnetic generator 304 or 412 to cause the electromagnetic generator to produce electromagnetic energy. The controller 210, 310, or 420 can comprise suitable processing circuitry configured to generate control signals representative of a desired acoustic signature to be produced in the treatment fluid. The control signals can be selected to cause the electromagnetic generator 304 or 412 to produce electromagnetic energy. The generated electromagnetic energy can cause the electromagnetically responsive material to move at a frequency (or a plurality of frequencies) that corresponds to information in the control signal sent to the electromagnetic generator 304 or 412. Movement of the electromagnetically responsive material 308 or 418 can result in the production of acoustic waves in a fluid, such as a treatment fluid, having a desired acoustic signature.

As described herein, in some embodiments, the electromagnetic generator can include a magnetic field generator. The controller 210, 310, or 420 can control, or generate, the current and voltage supplied to the electromagnetic generator 412. In some embodiments, the controller 210, 310, or 420 can generate current in the electromagnetic generator 304 or 412 at any suitable frequency and amperage. For example, the controller 210, 310, or 420 can generate the current to create a corresponding changing magnetic field at one or a plurality of frequencies and/or pulsation patterns that can correspond to a desired acoustic signature. For example, in some embodiments, the magnetic field patterns can be generated across a broad band of frequencies and at a plurality of frequencies.

### Examples of Handpieces

FIGS. 6-12 depict various embodiments of treatment instruments or apparatuses comprising handpieces that can used to generate and convert electromagnetic energy to acoustic waves in a fluid by way of an electromagnetically responsive medium. Although the illustrated embodiments include treatment instruments comprising a handpiece, in some embodiments, the treatment instrument can comprise a cap or other instrument that can be attached to the tooth.

FIG. 6 is a schematic diagram of another embodiment of a handpiece 400. As depicted in FIG. 6, the pressure wave generator 410 is positioned within a handpiece 400. The handpiece 400 can include a body 402, a fluid supply 404, the pressure wave generator 410, and an insulator 430.

In the embodiment of FIG. 6, the pressure wave generator 410 can include the electromagnetic generator 412, the electromagnetically responsive material 418, the volume 414, and the diaphragm 416.

As shown in FIG. 6, the fluid supply 404 can comprise a fluid passage. The electromagnetic generator 412 is positioned around the fluid supply 404. In some embodiments, the electromagnetic generator 412 contacts the outer body of the fluid supply 404. In other embodiments, the electromagnetic generator 412 does not contact the outer body of the fluid supply 404 and is positioned at any suitable distance away from the fluid supply 404.

As shown in FIG. 6, pressure wave generator 410 can be positioned upstream of and/or proximal to an inlet to a treatment region of a tooth, such as, for example, an opening at distal end of the fluid supply 404. In some embodiments, as shown in FIG. 14, a fluid platform can be provided at a distal end of the handpiece 400. The fluid platform can include a chamber configured to retain treatment fluid in the tooth. In some embodiments, pressure wave generator 410 can be positioned proximal to an opening between a fluid passage, such as fluid supply 410, and the chamber of a fluid platform.

As shown in FIG. 6, the electromagnetic generator 412 is coupled to a controller 420 by wires 422. The wires 422 extend out of the electromagnetic generator 412 and can be routed through the body 402 of the handpiece 400 to connect electrically to the controller 420. As shown in FIG. 6, the volume 414 containing the electromagnetically responsive material 418 can positioned within the fluid supply 404. The volume 414 containing the electromagnetically responsive material 418 can be held in place within the fluid supply 404 through various mechanical or chemical arrangements. For example, the volume 414 can be fastened or bonded to the inside of the fluid supply 404. Alternatively, in some embodiments, the volume 414 can be positioned on the outside of the fluid supply 404. In some embodiments, the volume 414 does not directly contact the treatment fluid 406. Alternatively, in some embodiments, the volume 414 can be positioned within or outside the diameter of the electromagnetic generator 412. The diaphragm 416 can be coupled to, exposed to, and/or forms part of the volume 414. The diaphragm 416 can be positioned such that the diaphragm 416 contacts the treatment fluid 406 inside the fluid supply 404. For example, in some embodiments, the diaphragm 416 can be fastened or bonded to the outside of the volume 414 which is installed within the fluid supply 404. In some emobdiments, the diaphragm 416 can be installed on one side of the volume 414 and positioned within a cut out section of the fluid supply 404. In some embodiments, the diaphragm 416 does not directly contact the treatment fluid 406, but instead contacts another component of the handpiece 400, such as the fluid supply 404. In some embodiments, the diaphragm 416 is positioned within a cutout section or opening of the volume 414 or forms a portion of the volume 414 such that the diaphragm contacts the electromagnetically responsive material 418.

In some embodiments, the insulator 430 can provide electrical isolation between a patient/operator of the handpiece 400 and the electromagnetic generator 412. Electrical isolation may be required for meeting regulatory emissions and susceptibility requirements. The insulator 430 can provide regulatory safety for the use of high voltage in the electromagnetic generator.

During operation, treatment fluid 406 flows through the fluid supply 404 along a flow path 408. The controller 420 generates electromagnetic energy by sending a suitable control signal through the electromagnetic generator 412. The generated electromagnetic energy can cause the electromagnetically responsive material 418 to move at one or multiple fluid frequencies that corresponds to information in the control signal sent through the electromagnetic generator 412. For example, the electromagnetically responsive material 418 can be moved back and forth in a wave-like pattern at multiple frequencies. This movement of the electromagnetically responsive material 418 can generate vibrations in the diaphragm 416 at various frequencies. Due to the incompressible nature of the treatment fluid 406, the vibration of the diaphragm 416 can create corresponding fluid motion and/or pressure waves in the treatment fluid 406. This fluid motion and/or pressure waves travel throughout the treatment fluid 406 as the treatment fluid 406 is delivered to the treatment region. In some embodiments, the pressure waves generated in the treatment fluid 406 can propagate throughout the treatment region to treat the tooth (for example, to clean or fill the treatment region of the tooth). As shown in FIG. 6, in some embodiments, the volume 414 can be arranged to produce a pressure wave vector parallel or generally parallel to a flow of treatment fluid in the fluid supply 404. Such an arrangement may provide efficiency for power transfer in a direction towards the treatment region.

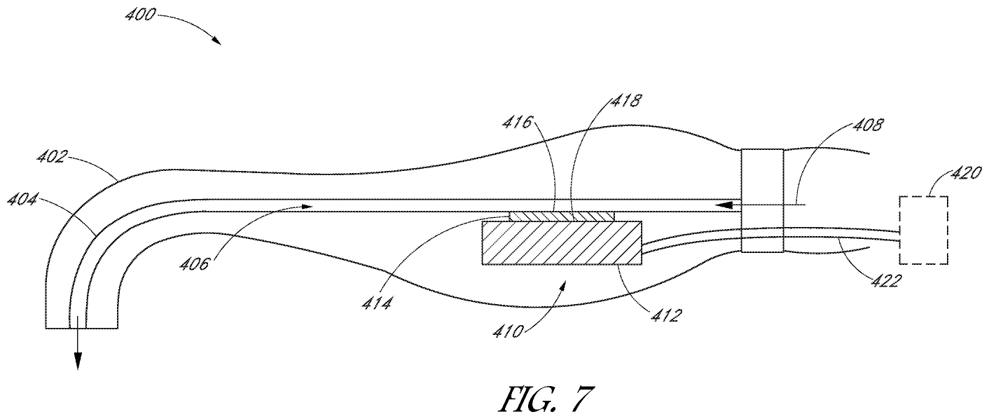

FIG. 7 is a schematic diagram of another embodiment of the handpiece 400 depicting an alternative arrangement for the pressure wave generator 410 installed within the handpiece 400. In FIG. 7 the electromagnetic generator 412 is installed off to a side of the fluid supply 404. The electromagnetic generator 412 is coupled to one side of the volume 414 containing the electromagnetically responsive material 418. The electromagnetic generator 412 can be coupled to the volume 414 through various mechanical or chemical connections, such as fastening or bonding. A first side of the diaphragm 416 is coupled to, exposed to, or forms at least a part of the ferrofluid volume 414 opposite from the electromagnetic generator 412. A second side of the diaphragm 416 couples to, is exposed to, or forms a part of the fluid supply 404. In some embodiments, the diaphragm 416 is positioned within a cutout of the fluid supply 404 such that the diaphragm 416 seals the fluid supply 404 and contacts any fluid within the fluid supply 404.

As shown in FIG. 7, pressure wave generator 410 can be positioned upstream of and/or proximal to an inlet to a treatment region of a tooth, such as, for example, an opening at distal end of the fluid supply 404. In some embodiments, as shown in FIG. 14, a fluid platform can be provided at a distal end of the handpiece 400. The fluid platform can include a chamber configured to retain treatment fluid in the tooth. In some embodiments, pressure wave generator 410 can be positioned proximal to an opening between a fluid passage, such as fluid supply 410, and the chamber of a fluid platform.

As shown in FIG. 7, in some embodiments, the volume 414 can be arranged to produce a pressure wave vector at an angle to, perpendicular to, or generally perpendicular to a flow of treatment fluid in the fluid supply 404. Such an arrangement may provide increased reflection and a high variety of incident angles, for example, in comparison to the arrangement of FIG. 6.

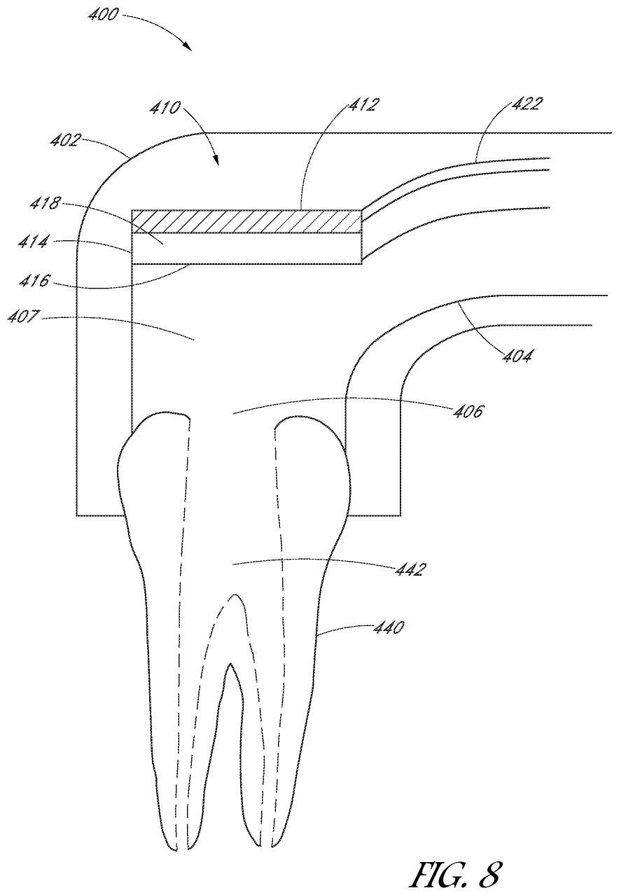

FIG. 8 is a schematic diagram of another embodiment of the handpiece 400 depicting an alternative arrangement for the pressure wave generator 410 installed within the handpiece 400. As depicted in FIG. 8, the pressure wave generator 410 is positioned adjacent a fluid supply 404 near a distal end of the handpiece 400. At this location, the pressure wave generator 410 can be located above or below a tooth 440 and a treatment region 442 while the handpiece 400 is in use. In some embodiments, the pressure wave generator 410 can be positioned opposite an access opening of the tooth 440. As shown in FIG. 8, in some embodiments, the fluid supply 404 can include a chamber 407 of the handpiece 400. In some embodiments, the pressure wave generator 410 can be positioned above the chamber 407. In use, the chamber 407 can be filed with treatment fluid 406 such that the treatment fluid 406 contacts the diaphragm 416 and fills the treatment region 442. The chamber 407 can be positioned above the treatment region 442 while in use, such that pressure waves created from the pressure wave generator 410 can travel directly to the treatment region 442.

In comparison to the arrangements of FIGS. 6 and 7, the pressure wave generator 410 in the arrangement of FIG. 8 can be positioned at a closer location relative to the treatment region. In some embodiments, the efficiency of acoustic energy transfer can increase as a distance between the pressure wave generator 410 and the treatment region decreases.

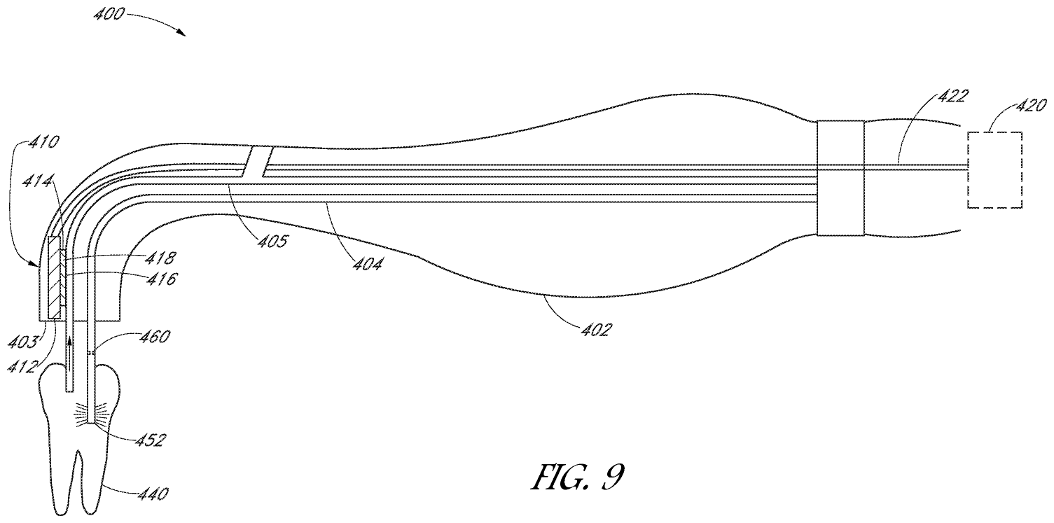

FIG. 9 is a schematic diagram of another embodiment of the handpiece 400 depicting an alternative arrangement for the pressure wave generator 410 installed within the handpiece 400. As illustrated in FIG. 9, the pressure wave generator 410 is positioned along an evacuation line or path 405. Although FIG. 9 depicts the pressure wave generator 410 installed adjacent to an end 403 of handpiece 400, in some embodiments, the pressure wave generator 410 can be installed at any point along the evacuation path 405.

In some embodiments, the pressure waves generated by the pressure wave generator 410 can travel against the flow of the treatment fluid and be delivered to the treatment region. In some embodiments, the arrangement of FIG. 9 can provide for a continuous flow or column of fluid from the tooth into alignment with the volume 414 that is air, bubble, and/or gas free. The air, bubble and/or gas free nature of the fluid evacuated from the tooth may enhance the propagation of pressure waves through the fluid.

As shown in FIG. 9, in some embodiments, pressure wave generator 410 can be positioned downstream of and/or proximal to an outlet of a treatment region of a tooth, such as, for example, an opening at distal end of the evacuation line or path 405. In some embodiments, as shown in FIG. 14, a fluid platform can be provided at a distal end of the handpiece 400. The fluid platform can include a chamber configured to retain treatment fluid in the tooth. In some embodiments, pressure wave generator 410 can be positioned proximal to an opening between a fluid passage, such as the evacuation line or path 405, and the chamber of a fluid platform.

As shown in FIG. 9, in some embodiments, the handpiece 400 can additionally include other components to assist with treating a tooth. For example, in FIG. 9, the handpiece 400 includes a nozzle 460 which creates a high pressure jet 452. As described in other embodiments herein, the high pressure jet 452 can assist with treating or cleaning the tooth 440. For example, in some embodiments, the high pressure jet 452 can act as a pressure wave generator or fluid motion generator. While utilizing the high pressure jet 452 to treat the tooth 440, the handpiece 400 can also utilize the pressure wave generator 410 to treat the tooth 440. In some embodiments, the pressure wave generator 410 can be operated independently of the high pressure jet 452. Accordingly, the pressure wave generator 410 can be operated at the same time that the high pressure jet 452 is being utilized, or can be utilized while the high pressure jet 452 is not in use or vice versa. In some embodiments, use of both the pressure wave generator 410 and high pressure liquid jet 452 can provide for increased efficacy and/or faster treatment times. Although FIG. 9 depicts a pressure wave generator 410 being utilized with a high pressure jet 452, the pressure wave generator 410 can be installed and utilized in other treatment devices described in other embodiments herein.

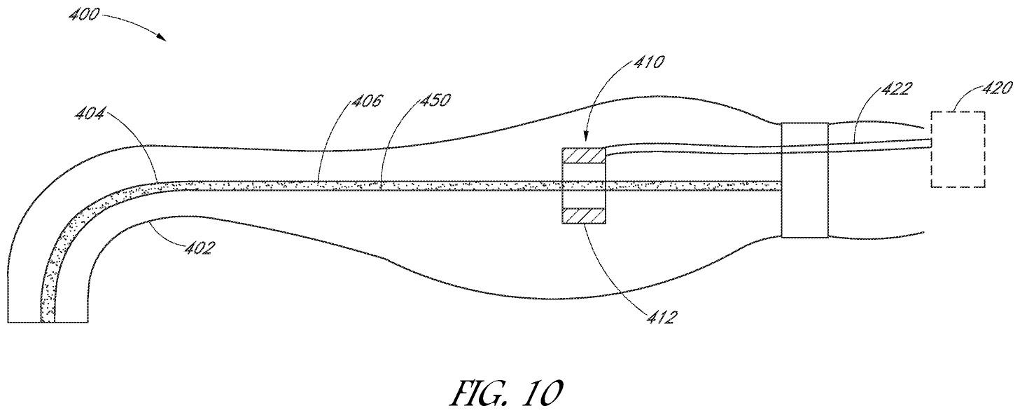

FIGS. 10-13 depict embodiments in which an electromagnetically responsive material is mixed with, suspended in, or part of the treatment fluid 406. In some embodiments, including an electromagnetically responsive material with the treatment fluid 406 can provide for higher energy transfer efficiency in comparison to embodiments in which diaphragms are utilized by eliminating efficiency losses associated with the diaphragms.

FIG. 10 is a schematic diagram of another embodiment of the handpiece 400 depicting an alternative arrangement for the pressure wave generator 410 installed within the handpiece 400. In FIG. 10, a plurality of particles 450 of an electromagnetically responsive material are suspended within the treatment fluid 406. In some embodiments, the electromagnetic generator 412 can be installed around the fluid supply 404. In some embodiments, the electromagnetic generator 412 does not contact the fluid supply 404 and a gap is present between the electromagnetic generator 412 and the fluid supply 404. In some embodiments, the electromagnetic generator 412 can contact the fluid supply 404. As shown in FIG. 10, the electromagnetic generator 412 can be connected to a controller 420 through wires 422.

As shown in FIG. 10, pressure wave generator 410 can be positioned upstream of and/or proximal to an inlet to a treatment region of a tooth, such as, for example, an opening at distal end of the fluid supply 404. In some embodiments, as shown in FIG. 14, a fluid platform can be provided at a distal end of the handpiece 400. The fluid platform can include a chamber configured to retain treatment fluid in the tooth. In some embodiments, pressure wave generator 410 can be positioned proximal to an opening between a fluid passage, such as fluid supply 410, and the chamber of a fluid platform.

During operation, treatment fluid 406 containing particles 450 flows through the fluid supply 404. The controller 420 generates electromagnetic energy by sending a suitable control signal through the electromagnetic generator 412. The generated electromagnetic energy can cause the particles 450 suspended within the treatment fluid 406 to move at one or multiple fluid frequencies that corresponds to the control signal sent through the electromagnetic generator 412. For example, the particles 450 can be moved back and forth in a wave-like pattern at multiple frequencies. This movement of the particles 450 can generate pressure waves within the treatment fluid 406 at various frequencies. These pressure waves travel throughout the treatment fluid 406 as the treatment fluid is delivered to the treatment region. In some embodiments, the pressure waves generated in the treatment fluid 406 can propagate throughout the treatment region to treat the tooth.

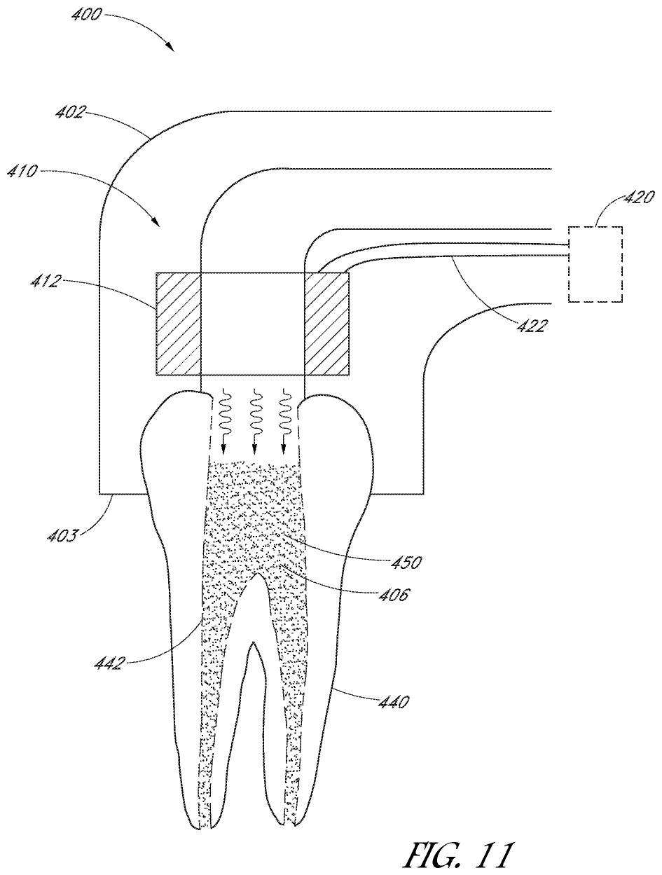

FIG. 11 is a schematic diagram of another embodiment of the handpiece 400 depicting an alternative arrangement for the pressure wave generator 410 installed within the handpiece 400. In FIG. 11, a plurality of particles 450 of an electromagnetically responsive material are suspended within the treatment fluid 406. As depicted in FIG. 11, in some embodiments, the electromagnetic generator 412 is installed near the end 403 of the handpiece 400. Accordingly, when the handpiece 400 is in use, the magnetic field generator 412 can be positioned above or below the treatment region 442 of the treated tooth 440. While the treatment region 442 is filled with treatment fluid 406 containing particles 450, the electromagnetic generator 412 can generate electromagnetic energy, causing the ferrous particles to create pressure waves within the treatment region 442.

In comparison to the arrangement of FIG. 10, the pressure wave generator 410 in the arrangement of FIG. 11 is positioned at a closer distance to the treatment region of the tooth. Such an arrangement may allow for minimization of losses to due distance between the electromagnetic generator 412 and the particles of the electromagnetically responsive material 450.

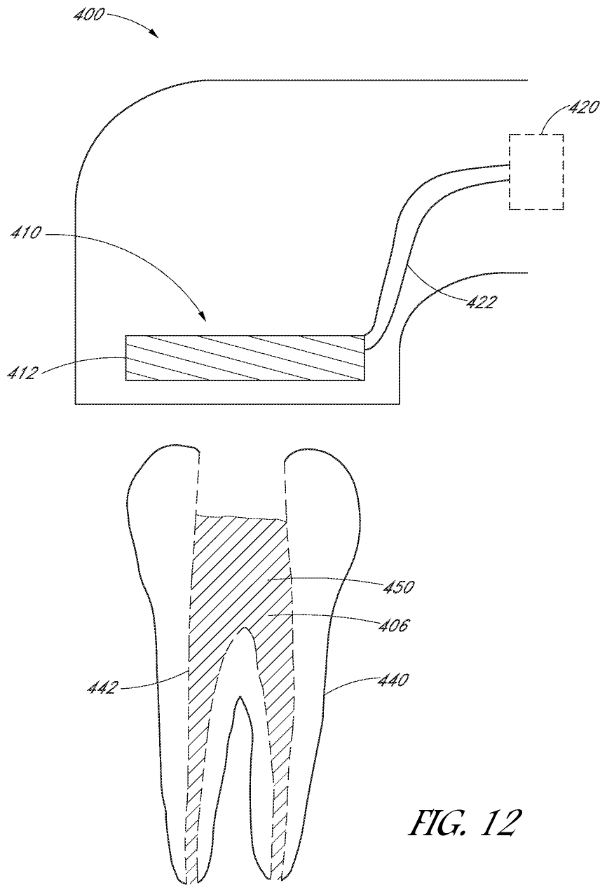

FIG. 12 is a schematic diagram of another embodiment of a handpiece 400 having a pressure wave generator 410 installed therein. In FIG. 12, a plurality of particles 450 of an electromagnetically responsive material are suspended within the treatment fluid 406. As shown in FIG. 12, in some embodiments, the electromagnetic generator 412 can be positioned within a handpiece 400 that is spaced apart from and/or otherwise not coupled with the tooth. As depicted in FIG. 12, the handpiece 400 can contain the electromagnetic generator 412, the controller 420, and the wires 422 connecting the controller 420 to the electromagnetic generator 412.

In some embodiments, the handpiece 400 may not contain a fluid supply 404. Instead, treatment fluid 406 containing ferrous particles 450 can be delivered to the treatment region 442 separately, for example, via a syringe or separate handpiece. In use, once the treatment region 442 is filled with treatment fluid 406 containing particles 450, handpiece 400 can be positioned above, below or at any other suitable positioned relative to the treatment region, and the magnetic field generator can generate a magnetic field to treat the tooth 440. The arrangement of FIG. 12 may provide a higher level of isolation between the patient and the electromagnetic generator 412 in comparison to the arrangements of FIGS. 6-11.

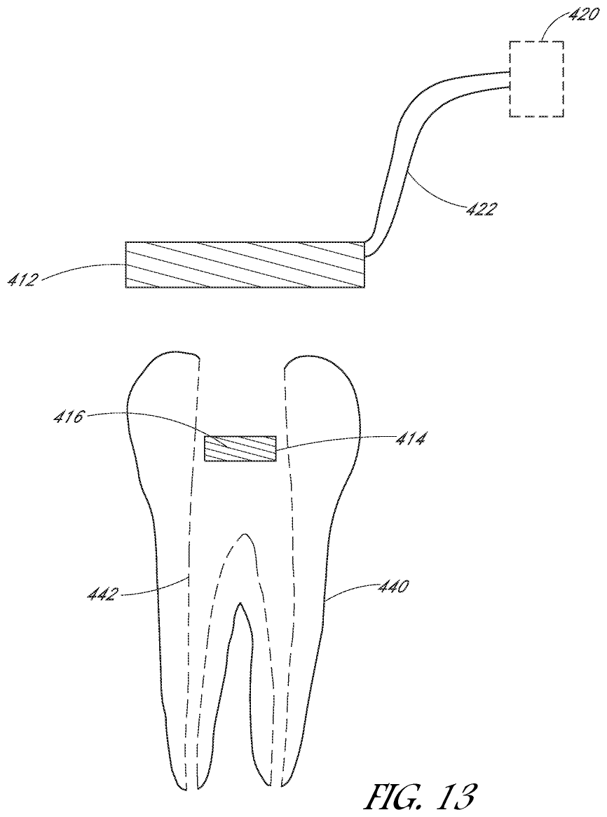

FIG. 13 is a schematic diagram of a system having an electromagnetic generator 412 and a volume 414. As shown in FIG. 13, the volume 414 is positioned within the treatment region 442 of the tooth 440. As shown in FIG. 13, the electromagnetic generator 412 is spaced apart from the volume 414. In some embodiments, the electromagnetic generator 412 can be housed separately from the volume 414. The electromagnetic generator 412 and volume 414 can be separate components without a physical link structurally attaching the two together. For example, the electromagnetic generator 412 can be housed in a handpiece 400 separate from the volume 414, or a treatment cap separate from the volume 414. The volume 414 can be a freestanding structure or part of another structure separate from the electromagnetic generator 412. In such embodiments, the volume 414 can be placed within the treatment region 442 of the tooth 440, and the electromagnetic generator 412 can be positioned at a location relative to the volume 414 sufficient to cause movement of the electromagnetically responsive material 418 within the volume 414.

In some embodiments, the electromagnetic generator 412 can be positioned outside of the tooth. For example, in some embodiments, the electromagnetic generator 412 can be positioned in a handpiece 400 at a location outside of the tooth. In other embodiments, the electromagnetic generator 412 can be positioned within the treatment region 442 of the tooth 440. For example, the electromagnetic generator 412 can be positioned within a handpiece 400 in a portion of the handpiece 400 that extends into the treatment region 442 of the tooth 440. Accordingly, various embodiments disclose a kit for treating a tooth that includes the electromagnetic generator 412 and the volume 414.

Although several of the figures depict use of the handpieces for treatment of a root canal, it is contemplated that the embodiments described herein can be used for any suitable dental treatment, including, for example, treatment of a carious region on an external surface of the tooth, cleaning of undesirable or unhealthy materials or deposits from exterior surfaces of teeth and gum tissue, etc. In some embodiments, the pressure wave generators described herein can be positioned adjacent to the external surfaces of the tooth during a treatment procedure, either in contact with the external surfaces of the tooth or spaced apart therefrom. In some embodiments, at least a portion of the pressure wave generators can be positioned within the tooth during a treatment procedure.

The apparatuses, systems, and methods described herein can be used in conjuction with dental cleaning procures, filling procedures, cosmetic procedures, such as tooth bleaching or whitening, etc. Treatment fluids can include treatment fluids, obturation materials, bleaching or whitening agents, or any other suitable fluids. In some embodiments, the treatment fluids can include degassed treatment fluids.

In some embodiments, the pressure waves can propagate through a cleaning fluid to clean the treatment location of the tooth (e.g., a root canal or carious region). In other embodiments, the pressure waves can propagate through an obturation material to fill the treatment region. In some embodiments, the pressure wave generator 410 can be used in conjunction with degassed treatment fluids to improve treatment outcomes. Moreover, the pressure wave generator 410 can be configured to generate a broad spectrum of energy across multiple frequencies to improve treatment outcomes. Beneficially, the embodiments disclosed herein can generate pressure waves having a desired acoustic signature that can be created by altering the magnetic fields with a controller 420.

Although the figures described herein depict a single pressure wave generator 410, it is contemplated that in certain embodiments, multiple pressure wave generators 410 can be used in a single treatment procedure. The multiple pressure wave generators 410 can be used alternatively or simultaneously. In some embodiments, different pressure wave generator may be placed at different locations relative to the treatment region of the tooth. In some embodiments, a handpiece 400 can include multiple pressure wave generators 410 that can be used alternatively or simultaneously. In some embodiments, multiple handpieces 400 may be used in a single treatment procedure. The multiple handpieces 400 may be used alternatively or simultaneously.

### Examples of Fluid Platforms for Fluid Management

Various embodiments disclosed herein may perform more efficiently if at least a portion of the pulp cavity of the tooth under treatment is filled with fluid (e.g., liquid) during a dental treatment procedure, such as an endodontic procedure. In some such treatment methods, the pulp chamber may be substantially filled with liquid with substantially no air (or gas) pockets remaining in the pulp chamber. For example, leakage of air into the pulp chamber may reduce the effectiveness of the treatment in some circumstances (e.g., by reducing the effectiveness of cavitation and damping the pressure waves). In some treatment methods, leakage of the fluid from the pulp chamber into the oral cavity (e.g., mouth) is not desired as such leakage may leave an unpleasant taste or smell or may lead to damaged tissues in the patient's mouth. Accordingly, in various treatment methods, a fluid platform can be used that maintains a substantially liquid-filled pulp chamber, inhibits leakage of air into the pulp chamber during treatment, and/or inhibits leakage of treatment fluid, waste fluid, and/or material from the pulp cavity into the mouth of the patient.

The fluid platform (e.g., a fluid retainer) can be used for maintaining fluid in a tooth chamber in a tooth, which may advantageously enable cleaning of a root canal space (or other portions of the tooth). In some procedures, fluid is delivered to the tooth chamber, and the fluid pressure in the tooth chamber may rise. If the fluid pressure in the chamber becomes too great, organic material, fluid, etc. may be forced through the apex of the tooth, which may lead to complications such as infection. Also, if for example due to suction negative pressure is created inside the tooth chamber, and if the absolute magnitude of the negative pressure is large enough, the negative pressure may cause problems such as pain and discomfort for the patient. Thus, in various embodiments, the fluid platform is configured such that the pressure created at the apex of the tooth (or in a portion of the tooth chamber such as, e.g., the pulp chamber) is below an upper value of: about 500 mmHg, about 300 mmHg, about 200 mmHg, about 100 mmHg, about 50 mmHg, about 30 mmHg, about 20 mmHg, or some other value. (Note: 1 mmHg is one millimeter of mercury and is a measure of pressure equal to about 133.322 Pascal). Embodiments of the fluid platform can be configured so that if the fluid pressure in the tooth chamber rises above an upper threshold, fluid can flow or leak from the chamber to maintain the fluid pressure at a safe or desired level. The threshold can be a predetermined pressure level. Certain predetermined pressure levels can be about 500 mmHg, about 300 mmHg, about 200 mmHg, about 100 mmHg, about 50 mmHg, about 30 mmHg, or about 20 mmHg.

In some implementations, it may be desired that the apical pressure or tooth chamber pressure be greater than a lower value of: about −1000 mmHg, about −500 mmHg, about −300 mmHg, about −200 mmHg, about −100 mmHg, about −50 mmHg, about 0 mmHg, or some other value. For example, if the pressure becomes too low (too negative), the patient may experience discomfort. The fluid retainer can be configured so that if the fluid pressure in the tooth chamber decreases below a lower threshold, ambient air can flow or be drawn through a flow restrictor (e.g., a sponge or vent) to maintain the fluid pressure above a patient-tolerable or desired level. The lower threshold can be a predetermined pressure level. Certain predetermined pressure levels can be about −1000 mmHg, about −500 mmHg, about −300 mmHg, about −200 mmHg, about −100 mmHg, about −50 mmHg, about or 0 mmHg. Thus, various embodiments of the fluid retainer can self-regulate the pressure in the tooth chamber to be below a first (e.g., upper) threshold and/or above a second (e.g., lower) threshold. As discussed, either or both thresholds can be a predetermined pressure level.

The fluid pressure in the tooth chamber may fluctuate with time as fluid flows in and out of the chamber and/or as a pressure wave generator is activated to generate acoustic waves (which comprise pressure oscillations). The acoustic waves may induce cavitation, which can cause pressure fluctuations as well. In some implementations, a mean or average pressure may be used. The mean pressure can be a time average of the pressure (at a particular point in the fluid) over a time period corresponding to the pressure fluctuations occurring in the fluid, or in some contexts, a spatial average of the pressure over a spatial region (e.g., over some or all of the tooth chamber). The pressure at a given point (in space or time) may be much larger than the mean pressure (e.g., due to a cavitation-induced event), and certain embodiments of the fluid platform may provide safety features to inhibit the rise of pressure above an undesired or unsafe threshold (e.g., by providing a vent to allow liquid to flow from the tooth chamber).

Although use of fluid platforms is described herein with respect to pulp chambers, in some embodiments, the embodiments of fluid platforms described herein can be used to retain fluid in any suitable treatment region.

In various treatment methods, when a fluid is delivered into a tooth chamber of a tooth, management of the fluid in the tooth chamber can be “controlled” or left “uncontrolled.”

### Examples of Uncontrolled Fluid Platforms

In some types of uncontrolled fluid platforms, the tooth chamber (e.g., a portion of the pulp cavity) may be substantially open to ambient air, fluids, etc., and the fluid inside the tooth chamber may not be fully contained in the tooth chamber. For example, the fluid may splash, overflow, or be evacuated via an external system (e.g., a suction wand) during the dental procedure. In some such cases, the fluid can be replenished intermittently or continuously during the procedure (e.g., via irrigation or syringing). The excess waste fluid also may be evacuated from the patient's mouth or from a rubber dam (if used) intermittently or continuously during the procedure.

An example of an uncontrolled method of fluid management can be the irrigation of the root canals with endodontic irrigation syringes. During this procedure, the fluid is injected into and exits from the pulp cavity, flowing into the oral space or a rubber dam (if used) and/or is suctioned by an external evacuation system operated by dental assistant. Another example of uncontrolled fluid management can be activation of the irrigation fluid by ultrasonic tips that can be inserted into the root canals. Upon activation of the ultrasonic device, the fluid in the tooth may splash out of the pulp cavity. The fluid inside the pulp cavity can be replenished via a syringe or the waterline of the ultrasonic tip, and the excess fluid may be suctioned from the oral space or the rubber dam (if used) via an external suction hose operated by a dental assistant.

### Examples of Controlled Fluid Platforms

Another type of fluid platform can be categorized as a “controlled” fluid platform. In some types of controlled fluid platforms, the fluid can be substantially contained in the tooth chamber (e.g., pulp cavity) by using an apparatus to at least partially cover an endodontic access opening. Some such fluid platforms may or may not include fluid inlets and/or outlets for the fluid to enter and exit the tooth chamber, respectively. Fluid flowing in and/or out of the tooth during a procedure can be controlled. In some embodiments, the total volume (or rate) of fluid going into the tooth can be controlled to be substantially equal to the total volume (or rate) of fluid going out of the tooth. Examples of two types of controlled fluid platforms will be described.

1. Examples of Closed Fluid Platforms

A closed system can be a controlled system where the amount of fluid flowing into the tooth chamber substantially equals the amount of fluid exiting the tooth chamber. An example of a closed system includes a fluid cap that is applied or sealed to the tooth, around the endodontic opening. In some such systems, the fluid's driving force (e.g., a pressure differential) is applied to only one of the openings (e.g., either inlet or outlet). In other implementations, the driving force can be applied at both the inlet and the outlet, in which case the applied driving forces may be regulated to be substantially equal in magnitude in order to reduce or avoid the following possible problems: exerting pressure (positive or negative) onto the tooth which may result in extrusion of fluid/debris periapically (e.g., positive pressure) or causing pain and/or bleeding due to excessive negative pressure, or breaking the seal of the fluid platform causing leakage of fluid and organic matter into the mouth (e.g., positive pressure) or drawing air into the chamber (e.g., negative pressure) which can reduce the treatment efficiency.

The operation of some closed fluid platforms can be relatively sensitive due to the regulation of the inlet and outlet fluid pressures to be substantially the same. Some such closed systems may lead to safety issues for the patient. For example, some such implementations may not ensure a substantially safe pressure that the patient's body can tolerate (e.g., apical pressures in a range from about −30 mmHg to +15 mmHg, or −100 mmHg to +50 mmHg, or −500 mmHg to +200 mmHg, in various cases). Some such closed systems can result in exertion of pressure (negative or positive) inside the tooth. For instance, if the driving force corresponds to the pressure at the inlet, a small obstruction on the outlet fluid line (which inhibits or reduces outflow of fluid from the tooth chamber) can result in increased pressure inside the tooth. Also, the elevation at which the waste fluid is discharged with respect to the tooth can cause static pressures inside the tooth.

2. Examples of Vented Fluid Platforms

Examples of a vented fluid platform include controlled systems where the inlet fluid flow rate and exit fluid flow rate may, but need not be, substantially the same. The two flow rates may in some cases, or for some time periods, be substantially the same. The fluid platform may include one or more “vents” that permit fluid to leave the tooth chamber, which can reduce the likelihood of an unsafe or undesired increase in fluid pressure (e.g., pressure at the periapical region). In some vented fluid platforms, the inlet and outlet flow rates may be driven by independent driving forces. For example, in some implementations, the fluid inlet can be in fluid communication with and driven by a pressure pump, while a fluid outlet can be in fluid communication with and controlled via an evacuation system (e.g., a suction or vacuum pump). In other implementations, the fluid inlet or outlet can be controlled with a syringe pump. The pressures of the fluid inlet and the fluid outlet may be such that a negative net pressure is maintained in the tooth chamber. Such a net negative pressure may assist delivering the treatment fluid into the tooth chamber from the fluid inlet.

In various embodiments described herein, the “vents” can take the form of a permeable or semi-permeable material (e.g., a sponge), openings, pores, or holes, etc. The use of vents in a controlled fluid platform may lead to one or more desirable advantages. For example, the evacuation system can collect waste fluid from the tooth chamber, as long as there is any available. If there is a pause in treatment (e.g. the time between treatment cycles), waste fluid flow may stop, and the evacuation system may start drawing air through the one or more vents to at least partially compensate for the lack of fluid supplied to the evacuation system, rather than depressurizing the tooth chamber. If the evacuation system stops working for any reason, the waste fluid may flow out through the one or more vents into the patient's mouth or onto a rubber dam (if used), where it can be collected by an external evacuation line. Therefore, the use of vent(s) can tend to dampen the effects of the applied pressure differential, and therefore may inhibit or prevent negative or positive pressure buildup inside the tooth. Certain embodiments of vented fluid platforms may provide increased safety since the system can be configured to maintain a safe operating pressure in the tooth, even when the operating parameters deviate from those specified. Also note that positive or negative pressure inside the tooth chamber can exert some amount of force on the sealing material(s), and as such a stronger seal may be required to withstand such force in some cases. Possible advantages of some vented systems include that the vent(s) help relieve pressure increases (or decreases) inside the tooth, reduce or eliminate the forces acting on the sealing material(s), and therefore render the sealing more feasible and effective.

In some embodiments, the fluid platform includes a fluid retainer (e.g., cap and flow restrictor). The fluid retainer may be used to retain fluid in a chamber in the tooth. The fluid retainer may include an internal (or inner) chamber such that when the fluid retainer is applied to the tooth, the internal chamber and the tooth chamber together form a fluid chamber. The fluid chamber may be at least partially filled with fluid. In some advantageous embodiments, the fluid chamber may be substantially or completely filled with fluid during a treatment procedure. The flow restrictor, which can function as the vent described above, may be used to permit fluid to flow from the chamber (e.g., if the fluid pressure in the chamber becomes too large) and/or to inhibit flow of air into the chamber. The flow restrictor can help retain fluid in the tooth chamber which may assist promoting fluid circulation in the tooth chamber, which may increase the effectiveness of irrigation or cleaning. The flow restrictor can comprise a sponge (e.g., an open-cell or closed-cell foam) in some embodiments.

The fluid platform also can include a fluid inlet for delivering fluid to the chamber in the tooth. The fluid inlet can have a distal end that may be configured to be submerged in the fluid in the chamber (after the chamber substantially fills with fluid). The distal end of the fluid inlet may be sized and shaped so that it can be disposed in the pulp chamber of the tooth. The distal end of the inlet may be disposed within the pulp chamber and above the entrances to the root canal spaces. Thus, in some such implementations, the fluid inlet does not extend into the canal spaces. In other implementations, the distal end of the inlet may be disposed in the fluid retained by the fluid retainer, but outside the pulp cavity (e.g., above the occlusal surface of the tooth). In some cases, the distal end of the fluid inlet can be sized/shaped to fit in a portion of a root canal space. For example, the distal end of the inlet may comprise a thin tube or needle. In various implementations, the inlet comprises a hollow tube, lumen, or channel that delivers the fluid to the tooth chamber. In other implementations, the fluid inlet may be a liquid beam (e.g., a high-velocity liquid jet) that is directed into the tooth chamber. In some such embodiments, the liquid beam may deliver fluid to the tooth chamber as well as generate pressure waves in the fluid in the chamber.

In some embodiments, the fluid platform can include a fluid introducer configured to supply fluid from a liquid source to the tooth chamber. The fluid introducer may comprise embodiments of the fluid inlet. In some implementations, the fluid introducer can also include a fluid line (or tubing) that provides fluidic communication between the fluid introducer and the liquid source. The fluid introducer may include a portion of a liquid jet device in some implementations.

The fluid inlet may be in fluid communication with a fluid reservoir, volume, supply, or source that provides the fluid to be delivered to the tooth via the inlet. The fluid may be delivered under pressure, for example, by use of one or more pumps or by using a gravity feed (e.g., by raising the height of the fluid volume above the height of the tooth chamber). The fluid platform may include additional components including, e.g., pressure regulators, pressure sensors, valves, etc. In some cases, a pressure sensor may be disposed in a tooth chamber, to measure the pressure in the tooth chamber during treatment.

The flow of fluid from the inlet may cause or augment fluid movement in the tooth chamber. For example, under various conditions of fluid inflow rate, pressure, inlet diameter, and so forth, the flow that is generated may cause (or augment) circulation, agitation, turbulence, etc. in the tooth chamber, which may improve irrigation or cleaning effectiveness in some cases. As described above, in some implementations a liquid jet device can be used to function as the inlet and can deliver fluid to the tooth chamber as well as generate pressure waves in the chamber. Thus, the liquid jet device can serve as the pressure wave generator and the fluid inlet in such implementations. The fluid from the liquid jet (as well as its conversion to a spray if an impingement plate is used) can induce circulation in the tooth chamber. The flow of fluid from the inlet can be used for a number of processes such as irrigation, cleaning, or disinfecting the tooth.

FIG. 14 schematically illustrates an example of a fluid platform 61 that can be used in a dental treatment procedure. The fluid platform 61 of FIG. 14 can be coupled with the examples of pressure wave generators (including electromagnetic generators and electromagnetically responsive materials) described above, and can support and position the pressure wave generators during a treatment procedure. The examples of pressure wave generators (including electromagnetic generators and electromagnetically responsive materials) -described herein can alternatively be coupled to other embodiments of fluid platforms described herein. In this example, the fluid platform 61 comprises a fluid retainer 66, a fluid inlet 71, and a fluid outlet 72 configured to remove fluid from a tooth chamber 65. In the illustrated embodiment, the fluid retainer 66 comprises a cap 70 that can be applied or attached to a tooth seal formed on the tooth. An (optional) flow restrictor 68 comprising elastic material (e.g., a sponge or semi-permeable material) can be disposed within the gap to assist in providing a substantially water tight seal between the cap 70 and the tooth seal 75. The substantially water tight seal helps retain fluid within the tooth chamber 65 during treatment and may also inhibit ambient air from entering the tooth chamber 65 during treatment.

In some implementations the fluid outlet 72 functions passively, for example, the fluid moves through the outlet 72 because of capillary forces, gravity, or a slight overpressure created in the tooth. In other implementations, the fluid outlet 72 is actively pumped, and the fluid can be transferred using a pump, suction, or other device that draws fluid out through the outflow conduit. In one example, the fluid outlet 72 comprises a suction line operated under partial vacuum pressure to suction out fluid and may be connected to the suction system/vacuum lines commonly found in a dental office.

In some embodiments, fluid may be at least partially retained in the fluid chamber 63, which can comprise the internal chamber 69 in the fluid retainer 66 and the tooth chamber 65. The fluid chamber 63 may be at least partially filled with fluid. In some advantageous embodiments, the fluid chamber 63 may be substantially or completely filled with fluid during a treatment procedure. During treatment, the fluid inlet 71 and the fluid outlet 72 can be in fluid communication with fluid retained in the fluid chamber 63. In the embodiment illustrated in FIG. 14, both the fluid inlet 71 and the fluid outlet 72 are in fluid communication with the fluid in the fluid chamber 63, and fluid can flow into the tooth from the fluid inlet 71 (solid arrowed lines 92a in FIG. 14) through an opening 411 in fluid communication with the chamber 63 and be removed from the tooth via the fluid outlet 72 (solid arrowed line 92b in FIG. 14). Note that in this embodiment, there is a single fluid chamber 63 in which both fluid delivered from the inlet 71 and fluid removed from the outlet 72 can directly fluidly communicate (e.g., without passing through a valve, a tube, a needle, etc.). In some embodiments, the delivery of fluid into the tooth chamber 65 via the fluid inlet 71 can cause a circulation in the tooth chamber 65 (see, e.g., arrowed lines 92a).

In this example, the fluid platform 61 comprises an additional flow restrictor in the form of a vent 73 that is disposed along the fluid outlet 72. The vent 73 can permit fluid from the tooth chamber 65 to flow out of the vent 73, for example if the fluid pressure becomes too large in the chamber. The vent 73 can act as a relief valve to inhibit over-pressurization of the tooth chamber 65.

In some embodiments, the vent 73 comprises a directionally biased valve that permits fluid to leave the tooth chamber 65 but inhibits ambient air from entering the tooth chamber 65. For example, the vent 73 may comprise one or more one-way (or check) valves. A one-way valve may have a cracking pressure selected to permit fluid to leave the tooth chamber 65 when the fluid pressure in the tooth chamber 65 exceeds a pressure threshold (e.g., about 100 mmHg in some cases). In other embodiments, a one-way valve may be used to permit ambient air to flow into the tooth chamber 65 when the pressure differential between ambient conditions and the pressure in the tooth chamber 65 is sufficiently large. For example, the cracking pressure of such a one-way valve may be selected such that if the fluid pressure in the chamber is less than a net (negative) threshold (e.g., the tooth chamber is under-pressurized), the valve will open to permit ambient air to flow into the fluid retainer 66. Such ambient air may be suctioned out of the fluid retainer 66 via a fluid outlet 72 (e.g., the one-way valve may be disposed along the fluid outflow line). In some embodiments, the vents 73 comprise a one-way valve to permit fluid to leave the fluid retainer 66 (while inhibiting ambient air from entering), and a one-way valve to permit ambient air to enter the fluid retainer 66. The cracking pressures of these two one-way valves may be selected so that in a desired pressure range, fluid is retained in the tooth chamber 65 and ambient air is inhibited from entering the tooth chamber 65. For example, the pressure range in the tooth may be between about −100 mmHg and +100 mmHg.

In other embodiments, the vent 73 may be configured to permit air to enter the fluid outlet 72 and be entrained with fluid removed from the tooth chamber 65. For example, as shown in FIG. 14, the vent 73 may be positioned and oriented such that ambient air flows into the fluid outlet 72 in the direction of the fluid flow in the outlet 72 (see, e.g., dashed arrowed line 94a). In such embodiments, the flow in the fluid outlet 72 includes both fluid from the tooth chamber 65 (see, e.g., solid arrowed line 92b) and ambient air (see, e.g., dashed arrowed line 94b). In some implementations, the vent 73 is disposed near the entry point of fluid into the outlet 72, e.g., within a few millimeters, which may make it easier for fluid to flow from the tooth chamber 65 if the pressure therein rises too high. In various embodiments, a plurality of vents 73 may be used such as, two, three, four, or more vents. The vents 73 may be sized, shaped, positioned, and/or oriented to allow fluid to flow from the tooth chamber 65 while inhibiting air from entering the tooth chamber 65.

The example system shown in FIG. 14 can assist in inducing fluid circulation in the tooth chamber 65 due to the inflow of fluid from the fluid inlet 71 and/or the removal of fluid from the fluid outlet 72 (if present). The example systems may also advantageously have patient safety features. For example, if the fluid outlet 72 is blocked (e.g., a suction tube is kinked or the suction ceases to function), the flow of fluid into the tooth chamber 65 from the inlet 71 can lead to increasing fluid pressures, which can lead to the level of fluid rising up into the outlet 72. The flow restrictor 68 (e.g., a sponge or a vent) can relieve the fluid pressure by allowing fluid to leave the tooth chamber 65 (e.g., by flowing through the sponge or leaking out the vent). As another example, if the fluid inlet 71 is blocked (or ceases to function), the fluid outlet 72 may remove the fluid from the tooth chamber 65 and may lead to increasingly lower pressures in the tooth chamber 65. The flow restrictor 68 can tend to keep the pressure in the tooth 10 at a safe or desirable level by allowing ambient air to flow into the fluid outlet 72 to at least partially alleviate the depressurization of the tooth chamber 65. Thus, by allowing the pressure in the tooth chamber 65 to remain within safe or desirable bounds (e.g., above a lower pressure threshold and below an upper pressure threshold), certain such embodiments may provide advantages over closed fluid containers that do not include some form of fluid restrictor or pressure relief valve.

Accordingly, certain embodiments of the fluid platform 61 may be at least partially open to the ambient environment (e.g., via the flow restrictor 68) and may substantially allow the pressure in the tooth chamber 65 to self-regulate. An additional advantage of certain such embodiments can be that pressure regulators, pressure sensors, inlet/outlet control valves, etc. need not be used to monitor or regulate the pressure in the tooth chamber 65 under treatment, because the self-regulation of the flow restrictor 68 permits the pressure to remain within desired or safe levels. In other embodiments, pressure regulators, pressure sensors, and control valves may be used to provide additional control over the fluid environment in the tooth. For example, pressure sensor(s) could be used to measure pressure along a fluid inlet 71 or a fluid outlet 72, in a portion of the tooth chamber 65, etc. In yet other embodiments, a temperature sensor or temperature controller may be used to monitor or regulate the temperature of the fluid in the fluid inlet 71 or a fluid outlet 72, in the tooth chamber 65, etc.

As shown in FIG. 14, a pressure wave generator 410 can be positioned along the fluid inlet 71. In some embodiments, the pressure wave generator 410 can be positioned along the fluid inlet 71 proximal to and upstream of the opening 411 at the distal end of the inlet 71 at which the inlet 71 opens to the fluid chamber 63. As shown in FIG. 14, the fluid inlet 71 can be in fluid communication with the internal chamber 69 when positioned against the tooth. In other embodiments, the fluid inlet 71 can terminate in the internal chamber 69 such that the opening 411 at the distal end of the inlet 71 is positioned outside of the tooth and exposed to the internal chamber 69.

As shown in FIG. 14, the pressure wave generator 410 is positioned outside of the tooth. The pressure wave generator 410 can be configured to generate pressure waves within the fluid inlet 71 that propagate through treatment fluid flowing through the inlet 71 and into the fluid chamber 63 and/or the fluid chamber 69 and to the treatment region of the tooth. In some embodiments, the pressure wave generator 410 can be positioned within the tooth but proximal to the opening 411. A fluid platform (which may be similar to the fluid platform 61 in some embodiments) comprising a chamber can be connected to the distal portion of the handpiece in the embodiments of FIGS. 2-13. An opening at the distal portion of the handpiece can communicate with the chamber of the fluid platform (such as fluid platform 61). As illustrated herein, the pressure wave generators can be positioned proximal the opening to the chamber of the fluid platform in various embodiments. In some embodiments, the opening comprises a nozzle configured to form a coherent collimated jet. In other embodiments, the opening may be wider so as to supply a stream of liquid to the chamber that may not be a jet.

In some embodiments, the fluid inlet 71, internal chamber 69, chamber 63, and/or fluid outlet 72 can serve as a fluid motion generator. As described above, the example system shown in FIG. 14 can assist in inducing fluid circulation in the tooth chamber 65 due to the inflow of fluid from the fluid inlet 71 and/or the removal of fluid from the fluid outlet 72 (if present). In some embodiments, one or more additional fluid motion generators can be employed within the system shown in FIG. 14, either in parallel or in series with the pressure wave generator 410.

In some embodiments, as shown for example in FIG. 9, a pressure wave generator 410 can be positioned along the fluid outlet 72 instead of or in addition to the pressure wave generator 410 in the fluid inlet 71. In some embodiments, a pressure wave generator 410 can be positioned within the internal chamber 69, instead of or in addition to pressure wave generators 410 in the fluid inlet 71 and/or fluid outlet 72. As described with respect to FIGS. 2 and 3, pressure wave generators 410 and fluid motion generators FMG can be positioned in parallel or in series within one or more of the inlet 71, the outlet 72, and the internal chamber 69.

Additional examples and details of fluid platforms can be found, for example, in column 9, line 5 to column 15, line 67 of U.S. Pat. No. 9,675,426 (“the '426 patent”), issued Jun. 13, 2017, which is incorporated by reference herein in its entirety and for all purposes. In some embodiments, the fluid platforms can comprise uncontrolled fluid platforms, as described at least in column 10, line 30 through column 10, line 57 of the '426 patent. Alternatively, or additionally, the fluid platforms can comprise controlled fluid platforms (such as vented fluid platforms), as described at least in column 10, line 58 through column 15, line 67 of the '426 patent.

3. Examples of Systems for Analyzing Fluid Leaving the Tooth

Substantially anything cleaned out from the pulpal chamber (in teeth that have pulpal chambers) and canals of a tooth (including pulp, debris, organic matter, calcified structures, etc.) can be monitored to determine the extent or progress of the tooth cleaning or to determine when the tooth becomes substantially clean. For example, when substantially no more pulp, calcified structures, organic matter, inorganic matter, and/or debris comes out of the tooth, the tooth may be substantially clean, and the system may provide a signal (e.g., audible/visible alarm, appropriate output on a display monitor) to the operator to stop the procedure. Such monitoring of the output from the tooth chamber can be used with any of the embodiments described herein, including with open, closed, or vented fluid platforms.

In some embodiments, a fluid platform can use an optional monitoring sensor. The monitoring sensor can monitor or analyze one or more properties of the fluid removed from the tooth. The monitoring sensor can include an optical, electrical (e.g., resistive), chemical, and/or electrochemical sensor. Monitoring sensors can include a liquid particle counter (e.g., configured to determine a range of particle sizes in the fluid), a liquid or gas chromatograph, a flame ionization detector, photoionization detector, a thermal conductivity detector, a mass spectrometer, etc. The monitoring sensor can use elemental analysis techniques to determine properties of the fluid.

In some embodiments, the monitoring sensor includes an optical sensor such as, e.g., a photometric sensor, a spectroscopic sensor, a color sensor, or a refractive index sensor. Optical properties in any part of the electromagnetic spectrum can be measured (e.g., ultraviolet, visible, infrared, etc.). For example, an optical sensor can include a light source (e.g., an LED) and a light detector (e.g., a photodiode) disposed relative to a fluid (e.g., fluid in the fluid outlet). The light source can emit light into the fluid and the light detector can measure the amount of light reflected from or transmitted through the fluid in the fluid outlet. At early stages of an endodontic treatment, the fluid from the tooth may contain substantial amounts of pulpal matter such that the fluid is murky and reflects, and does not transmit, much light. As the treatment proceeds, the amount of pulpal matter in the fluid decreases, and the reflectivity may correspondingly decrease (or the transmittivity may increase). When relatively little additional pulpal matter is contained in the fluid from the tooth, the fluid in the outlet may be substantially clear, and the reflectivity or transmittivity may reach a threshold value appropriate for fluid without pulpal matter (e.g., for clear water). The decrease of pulpal matter in the fluid outflow can be used as an indicator that the treatment is substantially complete or that the tooth chamber is substantially clean.

In some embodiments, a second monitoring sensor is disposed upstream of the fluid platform and can be used to provide a baseline measurement of properties of the fluid prior to entering the tooth chamber. For example, the threshold value may be based, at least in part, on the baseline measurement. Thus, in some embodiments, when the sensed property of the fluid property leaving the fluid platform is substantially the same as the sensed property of the fluid entering the fluid platform, it can be determined that the tooth treatment is substantially complete.

In various embodiments, the monitoring may be done continuously during the treatment or may be done at discrete times during the treatment. The monitoring sensor may be configured to measure an amount of carbon in the fluid, e.g., total organic carbon (TOC), total inorganic carbon, or total carbon. The amount of total inorganic carbon may reflect removal of hard structures such as calcified tissues, pulp stone, or dentin (e.g., tertiary dentin) during the treatment. The monitoring sensor may measure a property associated with removal of soft tissue (e.g., pulp, bacteria), hard tissue (e.g., pulp stone or calcified tissue), or both.

Thus when a property measured by the monitoring sensor reaches a threshold value, the system can alert the operator that the treatment is complete (e.g., little additional organic or inorganic material is being removed from the tooth). In some embodiments, a change in a measured property (e.g., a change between measurements at two different times) can be monitored, and when the change is sufficiently small (indicating that a threshold or plateau has been reached), the system can alert the operator that treatment is complete.

In some implementations, feedback from the monitoring sensor can be used to automatically adjust, regulate, or control one or more aspects of the endodontic treatment. For example, a tooth irrigation device, a tooth cleaning device, a fluid source, a fluid platform, a pressure wave generator, a fluid motion generator, etc. may be adjusted based on the feedback to automate some or all of the treatment. In one implementation, the concentration of a tissue dissolving agent (e.g., sodium hypochlorite) or a fluid flow rate can be adjusted based at least in part on feedback for a monitored amount of organic material in the tooth outflow. For example, if the amount of organic matter flowing from the tooth remains relatively high, the concentration of the tissue dissolving agent in the treatment fluid or the flow rate of the treatment fluid may be increased. Conversely, if the amount of organic matter decreases quickly, the tooth cleaning may be nearly complete, and the concentration of the tissue dissolver or the fluid flow rate may be decreased. In some such implementations, if the organic matter has decreased sufficiently, the system may switch to a different solute (e.g., a decalcifying agent) to begin a different phase of the treatment. In another implementation, feedback from the monitoring sensor can be used to adjust a pressure wave generator, for example, by increasing or decreasing the time the generator is activated (or deactivated). In some implementations using feedback, a proportional-integral-derivative (PID) controller or a fuzzy logic controller can be used to regulate or control aspects of the endodontic treatment.

4. Additional Features of Some Controlled Fluid Platforms

In some methods, little or substantially no treatment solution is injected through the apex of the tooth into the periapical region of the tooth (the tissues that surround the apex of the tooth). To limit injection of fluid into the periapical region, some embodiments are configured such that the pressure created inside the tooth and communicated to the apex of the tooth is equal to or lower than a pressure in the periapical region of the tooth that is tolerable by patients. In various embodiments, the fluid platform is configured such that the pressure created at the apex of the tooth (or in a portion of the tooth chamber such as, e.g., the pulp chamber) is below an upper value of about 500 mmHg, about 300 mmHg, about 200 mmHg, about 100 mmHg, about 50 mmHg, about 20 mmHg, or some other value. In some implementations, it may be desired that the apical pressure or tooth chamber pressure be above a lower value of about -1000 mmHg, about −500 mmHg, about −300 mmHg, about −200 mmHg, about −100 mmHg, about −50 mmHg, about 0 mmHg, or some other value. By selecting the size, number, and/or arrangement of fluid restrictors (e.g., sponges, vents, etc.), various systems can limit the apical pressure or the tooth chamber pressure to the foregoing values or ranges, as desired.

In some embodiments, it may be beneficial for the pressure at the apex of the tooth to be negative (e.g., lower than the pressure in the apical area). A negative pressure may allow inflamed bacteria, debris, and tissue (such as that found in a periapical lesion) to be suctioned out through the apex of the tooth and out of the mouth. It may be advantageous if the negative pressures created in the apex of the tooth are not too high (in magnitude) as this may induce pain in the patient. In one embodiment, the pressure created at the apex of the tooth is above about −1000 mmHg. In another embodiment, the pressure created at the apex of the tooth is above other values such as, e.g., about −600 mmHg, −500 mmHg, −250 mmHg, or some other value.

In some embodiments, substantially little or no treatment fluid, bacteria, tissue, debris, or chemicals enters the mouth during the procedure (e.g., substantially no leak from the handpiece and no leak between the handpiece and the tooth during the procedure), which may improve fluid management during the procedure. Spilling little or no material into the mouth during the procedure reduces the need to suction and remove waste fluid and material during the procedure. Accordingly, an assistant may not be needed during the procedure, which may simplify logistics and reduce manpower. Bacteria and debris removed from the infected tooth during the procedure should be avoided from being spilled into the mouth of the patient—so removing such material via the fluid platform may improve the cleanliness or hygiene of the procedure. Further, many of the chemicals used during endodontic procedures (e.g., NaOCl, etc.) may be corrosive or irritating to oral/gum tissue and reducing the likelihood of or preventing them from entering the patient's mouth is therefore desirable. Also, many of the chemicals and solutions used during endodontic procedures taste bad; therefore, not spilling such materials in the mouth during a procedure greatly improves patient comfort.

Delivering substances such as chemicals, medicaments, etc. in the treatment solution reduces the likelihood or prevents having to add such substances intermittently during an endodontic procedure (e.g. adding NaOCl intermittently during a root canal procedure). Embodiments of the fluid platform can allow one or more substances to be added during the procedure and in some implementations, the fluid can be automatically removed (e.g., via the fluid outlet). Substance concentration can be controlled or varied during procedure. One substance can be flushed out before introducing another substance, which may prevent unwanted chemical interactions. Embodiments in which the fluid platform is a closed system allow the use of more corrosive substances that may not be beneficial if spilled into the patient's oral environment. Substantially continuous replenishing of substances can help chemical reactions occur and may reduce the requirement for high concentration of such chemicals.

In various embodiments, a controlled fluid platform can be configured for one or more of the following. The fluid platform can allow analysis of fluid leaving the tooth to determine when procedure is complete. The fluid platform can prevent overheating of the tooth (if the pressure wave generator or other components generate heat) by irrigating the tooth chamber with fluid through the fluid inlet. The fluid platform can reduce or prevent air (e.g., gas) from being introduced into the tooth chamber, which may lower the effectiveness of irrigation, pressure waves, or cavitation. A controlled fluid platform can allow cleaning action/energy to be more effective during a procedure, e.g. fewer losses through mechanisms such as splashing, which removes both fluid mass and fluid momentum from the tooth chamber (which otherwise could provide circulation). The fluid platform can allow teeth to be treated in any orientation in space (e.g. upper or lower teeth may be treated while the patient reclines in a dental chair). The fluid platform can allow macroscopic circulation within the tooth to, for example, effectively remove tissue and debris from canals and canal spaces and/or effectively replenish new treatment solution.

### Additional Examples of Pressure Wave Generators

A pressure wave generator can be used in various embodiments to clean a tooth, e.g., from interior or exterior portions of the tooth and/or gums. In some embodiments, the pressure wave generator can be used to fill or obturate a cleaned root canal or other treatment region of the tooth. In some embodiments, a pressure wave generator can comprise an elongated member having an active distal end portion. The active distal end portion can be activated by a user to apply energy to the treatment tooth to remove unhealthy or undesirable material from the tooth.

In some embodiments, pressure wave generators can be configured to generate pressure waves and fluid motion with energy sufficient to clean undesirable material from a tooth. In various embodiments, a pressure wave generator can be a device that converts one form of energy into acoustic waves and/or bulk fluid motion (e.g., rotational motion) within the fluid. Pressure wave generators can induce, among other phenomena, both pressure waves and bulk fluid dynamic motion in a fluid (e.g., in the chamber), fluid circulation, turbulence, vortices and other conditions that can enable the cleaning of the tooth. Pressure wave generator can be used to clean the tooth by creating pressure waves that propagate through the fluid, e.g., through treatment fluid at least partially retained in a chamber. In some implementations, a pressure wave generator may also create cavitation, acoustic streaming, turbulence, etc. In various embodiments, a pressure wave generator can generate pressure waves or acoustic energy having a broadband power spectrum. For example, the pressure wave generator can generate pressure waves at multiple different frequencies, as opposed to only one or a few frequencies. Without being limited by theory, it is believed that the generation of power at multiple frequencies can help to remove various types of organic and/or inorganic materials that have different material or physical characteristics at various frequencies.

The pressure wave generators described herein (e.g., an electromagnetic generator, high-speed liquid jet, ultrasonic transducer, a laser fiber, etc.) can be placed at the desired treatment location in or on the tooth. The pressure wave generator can create pressure waves and fluid motion within the fluid inside a substantially-enclosed chamber. In general, the pressure wave generator can be sufficiently strong to remove unhealthy materials such as organic and/or inorganic tissue from teeth. In some embodiments, the pressure wave generator can be configured to avoid substantially breaking down or harming natural dentin and/or enamel.

1. Liquid Jet Apparatus

For example, in some embodiments, a pressure wave generator can comprise a liquid jet device. A liquid jet can be created by passing high pressure liquid through an orifice. A liquid jet can create pressure waves within the treatment liquid. In some embodiments, a pressure wave generator comprises a coherent, collimated jet of liquid. The jet of liquid can interact with liquid in a substantially-enclosed volume (e.g., the chamber and/or the mouth of the user) and/or an impingement member to create the acoustic waves. In addition, the interaction of the jet and the treatment fluid, as well as the interaction of the spray which results from hitting the impingement member and the treatment fluid, may assist in creating cavitation and/or other acoustic effects to clean the tooth.

In various embodiments, a liquid jet device can comprise a positioning member (e.g., a guide tube) having a channel or lumen along which or through which a liquid jet can propagate. The distal end portion of the positioning member can include one or more openings that permit the deflected liquid to exit the positioning member and interact with the surrounding environment in the chamber or tooth. In some treatment methods, the openings disposed at or near the distal end portion of the positioning member can be submerged in liquid that can be at least partially enclosed in the chamber attached to or enclosing a portion of the tooth. In some embodiments, the liquid jet can pass through the guide tube and can impact an impingement surface. The passage of the jet through the surrounding treatment fluid and impact of the jet on the impingement surface can generate the acoustic waves in some implementations. The flow of the submerged portion of the liquid jet may generate a cavitation cloud within the treatment fluid. The creation and collapse of the cavitation cloud may, in some cases, generate a substantial hydroacoustic field in or near the tooth. Further cavitation effects may be possible, including growth, oscillation, and collapse of cavitation bubbles. In addition, as explained above, bulk fluid motion, such as rotational flow, may be induced. The induced rotational flow can enhance the cleaning process by removing detached material and replenishing reactants for the cleaning reactions. These (and/or other) effects may lead to efficient cleaning of the tooth.

In some embodiments, a system for generating a high-velocity jet can include a motor, a motor controller, a fluid source, a pump, a pressure sensor , a system controller, a user interface, and a handpiece that can be operated by a dental practitioner to direct the jet toward desired locations in a patient's mouth. The pump can pressurize fluid received from the fluid source. The pump may comprise a piston pump in which the piston is actuatable by the motor. The motor can be controlled by way of the motor controller. The high-pressure liquid from the pump can be fed to the pressure sensor and then to the handpiece, for example, by a length of high-pressure tubing. The pressure sensor may be used to sense the pressure of the liquid and communicate pressure information to the system controller. The system controller can use the pressure information to make adjustments to the motor and/or the pump to provide a target pressure for the fluid delivered to the handpiece. For example, in embodiments in which the pump comprises a piston pump, the system controller may signal the motor to drive the piston more rapidly or more slowly, depending on the pressure information from the pressure sensor. In some embodiments, the pressure of the liquid that can be delivered to the handpiece can be adjusted within a range from about 500 psi to about 50,000 psi (1 psi is 1 pound per square inch and is about 6895 Pascal (Pa)). In certain embodiments, it has been found that a pressure range from about 2,000 psi to about 15,000 psi produces jets that are particularly effective for endodontic treatments. In some embodiments, the pressure is about 10,000 psi.

A fluid source may comprise a fluid container (e.g., an intravenous bag) holding any of the treatments fluids described herein. The treatment fluid may be degassed, with a dissolved gas content less than normal (e.g., non-degassed) fluids. Examples of treatment fluids include sterile water, a medical-grade saline solution, an antiseptic or antibiotic solution (e.g., sodium hypochlorite), a solution with chemicals or medications, or any combination thereof. More than one fluid source may be used. In certain embodiments, it is advantageous for jet formation if the liquid provided by the fluid source is substantially free of dissolved gases, which may reduce the effectiveness of the jet and the pressure wave generation. Therefore, in some embodiments, the fluid source comprises degassed liquid such as, e.g., degassed distilled water. A bubble detector (not shown) may be disposed between the fluid source and the pump to detect bubbles in the liquid and/or to determine whether liquid flow from the fluid source has been interrupted or the container has emptied. Also, as discussed above degassed fluids may be used. The bubble detector can be used to determine whether small air bubbles are present in the fluid that might negatively impact jet formation or acoustic wave propagation. Thus in some embodiments, a filter or de-bubbler (not shown) can be used to remove small air bubbles from the liquid. The liquid in the fluid source may be at room temperature or may be heated and/or cooled to a different temperature. For example, in some embodiments, the liquid in the fluid source can be chilled to reduce the temperature of the high velocity jet generated by the system, which may reduce or control the temperature of the fluid inside a tooth. In some treatment methods, the liquid in the fluid source can be heated, which may increase the rate of chemical reactions that may occur in the tooth during treatment.

A handpiece can be configured to receive a high pressure liquid and can be adapted at a distal end to generate a high-velocity beam or jet of liquid for use in dental procedures. In some embodiments, a dental treatment system may produce a coherent, collimated jet of liquid. A handpiece may be sized and shaped to be maneuverable in the mouth of a patient so that the jet may be directed toward or away from various portions of the tooth. In some embodiments, a handpiece comprises a distal housing or coupling member that can be coupled to the tooth.

A system controller may comprise a microprocessor, a special or general purpose computer, a floating point gate array, and/or a programmable logic device. The system controller may be used to control safety of the system, for example, by limiting system pressures to be below safety thresholds and/or by limiting the time that the jet is permitted to flow from the handpiece. A dental treatment system may also include a user interface that outputs relevant system data or accepts user input (e.g., a target pressure). In some embodiments, the user interface comprises a touch screen graphics display. In some embodiments, the user interface may include controls for a dental practitioner to operate the liquid jet apparatus. For example, the controls can include a foot switch to actuate or deactuate the jet. In some embodiments, the motor, motor controller, pump, fluid source, pressure sensor, system controller, and user interface can be integrated into a console.

A dental treatment system may include additional and/or different components and may be configured differently than described herein. For example, the system may include an aspiration pump that is coupled to the handpiece (or an aspiration cannula) to permit aspiration of organic matter from the mouth or tooth. In other embodiments, the system may comprise other pneumatic and/or hydraulic systems adapted to generate the high-velocity beam or jet.

Additional details of a pressure wave generator and/or pressure wave generator that includes a liquid jet device may be found at least in ¶¶ [0045]40050], [0054]-[0077] and various other portions of U.S. Patent Publication No. US 2011/0117517, published May 19, 2011, and in ¶¶ [0136]-[0142] and various other portions of U.S. Patent Publication No. US 2012/0237893, published Sep. 20, 2012, each of which is incorporated by reference herein in its entirety and for all purposes.

As has been described, a pressure wave generator can be any physical device or phenomenon that converts one form of energy into acoustic waves within the treatment fluid and that induces rotational fluid motion in the chamber and/or tooth. Many different types of pressure wave generators (or combinations of pressure wave generators) are usable with embodiments of the systems and methods disclosed herein.

2. Mechanical Energy

Mechanical energy pressure wave generators can also include rotating objects, e.g. miniature propellers, eccentrically-confined rotating cylinders, a perforated rotating disk, etc. These types of pressure wave generators can also include vibrating, oscillating, or pulsating objects such as sonication devices that create pressure waves via piezoelectricity, magnetostriction, etc. In some pressure wave generators, electric energy transferred to a piezoelectric transducer can produce acoustic waves in the treatment fluid. In some cases, the piezoelectric transducer can be used to create acoustic waves having a broad band of frequencies.

3. Electromagnetic Beam Energy

An electromagnetic beam of radiation (e.g., a laser beam) can propagate energy into a chamber, and the electromagnetic beam energy can be transformed into acoustic waves as it enters the treatment fluid. In some embodiments, the laser beam can be directed into the chamber as a collimated and coherent beam of light. The collimated laser beam can be sufficient to generate pressure waves as the laser beam delivers energy to the fluid. Furthermore, in various embodiments, the laser beam can be focused using one or more lenses or other focusing devices to concentrate the optical energy at a location in the treatment fluid. The concentrated energy can be transformed into pressure waves sufficient to clean the undesirable materials. In one embodiment, the wavelength of the laser beam or electromagnetic source can be selected to be highly absorbable by the treatment fluid in the chamber or mouth (e.g., water) and/or by the additives in the treatment fluid (e.g., nanoparticles, etc.). For example, at least some of the electromagnetic beam energy may be absorbed by the fluid (e.g., water) in the chamber, which can generate localized heating and pressure waves that propagate in the fluid. The pressure waves generated by the electromagnetic beam can generate photo-induced or photo-acoustic cavitation effects in the fluid. In some embodiments, the localized heating can induce rotational fluid flow in the chamber and/or tooth that further enhances cleaning of the tooth. The electromagnetic radiation from a radiation source (e.g., a laser) can be propagated to the chamber by an optical waveguide (e.g., an optical fiber), and dispersed into the fluid at a distal end of the waveguide (e.g., a shaped tip of the fiber, e.g., a conically-shaped tip). In other implementations, the radiation can be directed to the chamber by a beam scanning system.

The wavelength of the electromagnetic beam energy may be in a range that is strongly absorbed by water molecules. The wavelength may in a range from about 300 nm to about 3000 nm. In some embodiments, the wavelength is in a range from about 400 nm to about 700 nm, about 700 nm to about 1000 nm (e.g., 790 nm, 810 nm, 940 nm, or 980 nm), in a range from about 1 micron to about 3 microns (e.g., about 2.7 microns or 2.9 microns), or in a range from about 3 microns to about 30 microns (e.g., 9.4 microns or 10.6 microns). The electromagnetic beam energy can be in the ultraviolet, visible, near-infrared, mid-infrared, microwave, or longer wavelengths.

The electromagnetic beam energy can be pulsed or modulated (e.g., via a pulsed laser), for example with a repetition rate in a range from about 1 Hz to about 500 kHz. The pulse energy can be in a range from about 1 mJ to about 1000 mJ. The pulse width can be in a range from about 1 μs to about 500 μs, about 1 ms to about 500 ms, or some other range. In some cases, nanosecond pulsed lasers can be used with pulse rates in a range from about 100 ns to about 500 ns. The foregoing are non-limiting examples of radiation parameters, and other repetition rates, pulse widths, pulse energies, etc. can be used in other embodiments.

The laser can include one or more of a diode laser, a solid state laser, a fiber laser, an Er:YAG laser, an Er:YSGG laser, an Er,Cr:YAG laser, an Er,Cr:YSGG laser, a Ho:YAG laser, a Nd:YAG laser, a CTE:YAG laser, a CO₂ laser, or a Ti:Sapphire laser. In other embodiments, the source of electromagnetic radiation can include one or more light emitting diodes (LEDs). The electromagnetic radiation can be used to excite nanoparticles (e.g., light-absorbing gold nanorods or nanoshells) inside the treatment fluid, which may increase the efficiency of photo-induced cavitation in the fluid. The treatment fluid can include excitable functional groups (e.g., hydroxyl functional groups) that may be susceptible to excitation by the electromagnetic radiation and which may increase the efficiency of pressure wave generation (e.g., due to increased absorption of radiation). During some treatments, radiation having a first wavelength can be used (e.g., a wavelength strongly absorbed by the liquid, for instance water) followed by radiation having a second wavelength not equal to the first wavelength (e.g., a wavelength less strongly absorbed by water) but strongly absorbed by another element, e.g. dentin, or nanoparticles added to solution. For example, in some such treatments, the first wavelength may help create bubbles in the fluid, and the second wavelength may help disrupt the tissue.

The electromagnetic beam energy can be applied to the chamber for a treatment time that can be in a range from about one to a few seconds up to about one minute or longer. A treatment procedure can include one to ten (or more) cycles of applying electromagnetic beam energy to the tooth. A fluid can circulate or otherwise move in the chamber during the treatment process, which advantageously may inhibit heating of the tooth (which may cause discomfort to the patient). The movement or circulation of treatment fluid (e.g., water with a tissue dissolving agent) in the chamber can bring fresh treatment fluid to tissue and organic matter as well as flush out dissolved material from the treatment site. In some treatments using electromagnetic radiation, movement of the treatment fluid can increase the effectiveness of the cleaning (as compared to a treatment with little or no fluid circulation).

In some implementations, electromagnetic energy can be added to other fluid motion generation modalities. For example, electromagnetic energy can be delivered to a chamber in which another pressure wave generator (e.g., a liquid jet) is used to generate the acoustic waves.

4. Acoustic Energy

Acoustic energy (e.g., ultrasonic, sonic, audible, and/or lower frequencies) can be generated from electric energy transferred to, e.g., an ultrasound or other transducer or an ultrasonic tip (or file or needle) that creates acoustic waves in the treatment fluid. The ultrasonic or other type of acoustic transducer can comprise a piezoelectric crystal that physically oscillates in response to an electrical signal or a magnetostrictive element that converts electromagnetic energy into mechanical energy. The transducer can be disposed in the treatment fluid, for example, in the fluid inside the chamber. Ultrasonic or other acoustic devices used with the embodiments disclosed herein are preferably broadband and/or multi-frequency devices. For example, unlike the power spectra of the conventional ultrasonic transducer, ultrasonic or other acoustic devices used with the disclosed embodiments preferably have broadband characteristics.

5. Further Properties of Some Pressure Wave Generators

A pressure wave generator can be placed at a desired location with respect to the tooth. The pressure wave generator creates pressure waves within the fluid inside the chamber (the generation of acoustic waves may or may not create or cause cavitation). The acoustic or pressure waves propagate throughout the fluid inside the chamber, with the fluid in the chamber serving as a propagation medium for the pressure waves. The pressure waves can also propagate through tooth material (e.g., dentin). It is believed, although not required, that as a result of application of a sufficiently high-intensity acoustic wave, acoustic cavitation may occur. The collapse of cavitation bubbles may induce, cause, or be involved in a number of processes described herein such as, e.g., sonochemistry, tissue dissociation, tissue delamination, sonoporation, and/or removal of calcified structures. In some embodiments, apressure wave generator can be configured such that the acoustic waves (and/or cavitation) do not substantially break down natural dentin in the tooth. The acoustic wave field by itself or in addition to cavitation may be involved in one or more of the abovementioned processes.

In some implementations, a pressure wave generator generates primary cavitation, which creates acoustic waves, which may in turn lead to secondary cavitation. The secondary cavitation may be weaker than the primary cavitation and may be non-inertial cavitation. In other implementations, the pressure wave generator generates acoustic waves directly, which may lead to secondary cavitation.

The energy source that provides the energy for the pressure wave generator can be located outside the handpiece, inside the handpiece, integrated with the handpiece, etc.

Additional details of pressure wave generators (e.g., which may comprise a pressure wave generator) that may be suitable for use with the embodiments disclosed herein may be found, e.g., in ¶¶ [0191]-[0217], and various other portions of U.S. Patent Publication No. US 2012/0237893, published Sep. 20, 2012, which is incorporated by reference herein for all purposes.

Other pressure wave generators may be suitable for use with the disclosed embodiments. For example, a fluid inlet can be disposed at a distal portion of a handpiece and/or can be coupled to a fluid platform in some arrangements. The fluid inlet can be configured to create movement of the fluid in a chamber, turbulence in the fluid in the chamber, fluid motion of the fluid in the chamber and/or produce other dynamics in the fluid in the chamber. For example, the fluid inlet can inject fluid into or on the tooth to be treated. In addition, mechanical stirrers and other devices can be used to enhance fluid motion and cleaning. The fluid inlet can improve the circulation of the treatment fluid in a chamber, which can enhance the removal of unhealthy materials from the tooth. For example, as explained herein, faster mechanisms of reactant delivery such as “macroscopic” liquid circulation may be advantageous in some of the embodiments disclosed herein.

In some embodiments, multiple pressure wave generators can be disposed in or on the chamber. As explained herein, each of the multiple pressure wave generators can be configured to propagate acoustic waves at a different frequency or range of frequencies. For example, different acoustic frequencies can be used to remove different types of materials. The multiple pressure wave generators can be activated simultaneously and/or sequentially in various arrangements.

### Examples of Fluid Motion Generators

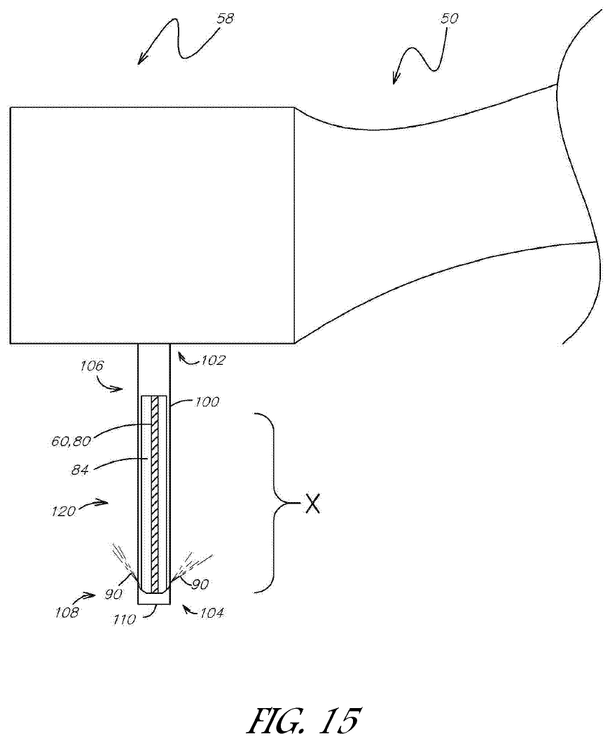

FIGS. 15-17 depict examples of fluid motion generators. Fluid motion generators can be configured to generate large-scale or bulk fluid motion as described herein.

In some embodiments, a fluid motion generator can include a liquid jet apparatus. For example, in some embodiments, a fluid motion generator can include a nozzle configured to form a high-velocity liquid jet. FIG. 15 schematically illustrates an example of a distal end 58 of an embodiment of a handpiece 50 comprising a fluid motion generator configured to generate fluid motion using a high-velocity liquid jet 60. The handpiece 60 includes a guide tube 100. The guide tube 100 has a proximal end 102 that can be attached or disposed adjacent the distal end 58 of the handpiece 50 and a distal end 104 that can be disposed in a treatment fluid. For example, the distal end 104 of the guide tube 100 can be disposed in a treatment region of the tooth. The guide tube 100 has a channel 84 that permits propagation of the liquid jet 60 along at least a portion of the length of the guide tube 100. For example, the liquid jet 60 may propagate along the longitudinal jet axis 80. In the embodiment schematically depicted in FIG. 15, the longitudinal jet axis 80 is substantially collinear with the longitudinal axis of the channel 84 and the guide tube 100. In other embodiments, the longitudinal jet axis 80 may be offset from the longitudinal axis of the channel 84 and/or the guide tube 100, for example, by offsetting an orifice of a nozzle forming the liquid jet relative to the axes of the channel 84 and/or guide tube 100.

In some embodiments, the proximal end 102 of the guide tube 100 can be attached to the distal end 58 of the dental handpiece 50. The liquid jet 60 (which may be a coherent, collimated jet) can propagate from the handpiece 50 along the jet axis 80, which can pass through the channel 84 of the guide tube 100.

In some embodiments, the guide tube 100 can be sized or shaped such that the distal end 104 can be positioned through an endodontic access opening formed in the tooth, for example, on an occlusal surface, a buccal surface, or a lingual surface. For example, the distal end 104 of the guide tube may be sized or shaped so that the distal end 104 can be positioned in the pulp cavity of the tooth, e.g., near the pulpal floor, near openings to the canal space, or inside the canal openings. The size of the distal end 104 of the guide tube 100 can be selected so that the distal end 104 fits through an access opening of the tooth.

As shown in FIG. 15, in some embodiment, the guide tube 100 can comprise an impingement member 110. The jet 60 can propagate along the channel 84 and impinge upon the impingement member 110, whereby at least a portion of the jet 60 can be slowed, disrupted or deflected, which can produce a spray 90 of liquid. The spray 90 may comprise droplets, beads, mist, jets, or beams of liquid in various implementations. In some embodiments, bulk fluid motion is generated via the spray 90 of liquid. In some embodiments, the liquid jet device forming the high velocity liquid jet 60 and the impingement member 110 can act as a fluid motion generator by generating bulk fluid motion within a treatment region of the tooth via the spray 90 of liquid, as described in further detail below.

Embodiments of the guide tube 100 which include an impingement member 110 may reduce or prevent possible damage that may be caused by the jet during certain dental treatments. For example, use of the impingement member 110 may reduce the likelihood that the jet may undesirably cut tissue or propagate into the root canal spaces 30 (which may undesirably pressurize the canal spaces in some cases). The design of the impingement member 110 may also enable a degree of control over the fluid circulation (or other bulk fluid motion) or pressure waves that can occur in the pulp cavity during treatment.

In use, the impingement member 110 may be disposed in a treatment region of the tooth. In some methods, the impingement member 110 is disposed in fluid in the tooth, and the liquid jet 60 impacts an impingement surface of the impingement member 110 while the impingement member 110 is disposed in the cavity. The liquid jet 60 may be generated in air or fluid, and in some cases, a portion of the liquid jet 60 passes through at least some (and possibly a substantial portion) of fluid in the treatment region of the tooth before impacting the impingement member 110. In some cases, the fluid in the treatment region may be relatively static; in other cases, the fluid in the treatment region may circulate, be turbulent, or have fluid velocities that are less than (or substantially less than) the speed of the high-velocity liquid jet.

The guide tube 100 can include an opening 120 that permits the spray 90 to leave the distal end 104 of the guide tube 100. In some embodiments, multiple openings 120 can be used. The opening 120 can have a proximal end 106 and a distal end 108. The distal end 108 of the opening 120 can be disposed near the distal end 104 of the guide tube 100. The opening 120 can expose the liquid jet 60 (and/or the spray 90) to the surrounding environment, which may include air, liquid, organic material, etc. For example, in some treatment methods, when the distal end 104 of the guide tube 100 is inserted into the treatment region, the opening 120 permits the material or fluid inside the treatment region to interact with the jet 60 or spray 90. A hydroacoustic field (e.g., pressure waves, acoustic energy, etc.) may be established in the tooth by the impingement of the jet 60 on the impingement member 110, interaction of the fluid or material in the tooth 10 with the jet 60 or the spray 90, fluid circulation or agitation generated in the pulp cavity, or by a combination of these factors (or other factors). The hydroacoustic field may include acoustic power over a relatively broad range of acoustic frequencies (e.g., from about a few kHz to several hundred kHz or higher). The hydroacoustic field in the tooth may influence, cause, or increase the strength of effects including, e.g., acoustic cavitation (e.g., bubble formation and collapse, microjet formation), fluid agitation, fluid circulation, sonoporation, sonochemistry, and so forth. It is believed, although not required, that the hydroacoustic field, some or all of the foregoing effects, or a combination thereof may act to disrupt or detach organic material in the tooth, which may effectively clean the pulp cavity and/or the canal spaces.

As described herein, in some embodiments, bulk fluid motion can be produced via the spray 90. As described herein, in some embodiments, upon impingement with the impingement member 110, at least a portion of the jet 60 can be slowed, disrupted or deflected to produce the spray 90 of liquid. As shown in FIG. 15, the spray 90 can be directed proximally towards the proximal end 102 of the guide tube. As the spray 90 is directed proximally through treatment fluid within the treatment region, the spray 90 can shear the surrounding fluid to create vortices (or other bulk fluid motion) in the treatment region. In some embodiments, the impingement member 110 can include one or more angled or curved sections that angle or curve back toward the direction of the oncoming jet 60 (e.g., away from the distal end 104 of the guide tube) and which may help direct some of the jet 60 (or the spray 90) back towards the proximal end 106 of the opening 120.

While placement of the impingement member 110 within a treatment region of the tooth is generally described with respect to FIG. 15, in some embodiments, a liquid jet device and/or an impingement member 110 can be positioned in other locations within a treatment device, such as a handpiece, to generate fluid motion. For example, in some embodiments, a liquid jet device and/or impingement member 110 can be positioned within a fluid passage, such as a fluid inlet or outlet, to generate bulk fluid motion within the fluid passage that propagates to a treatment region of the tooth. In some embodiments, a liquid jet device and/or impingement member 110 can be positioned in a chamber of a fluid platform to generate bulk fluid motion within the chamber that propagates to the treatment region of the tooth.

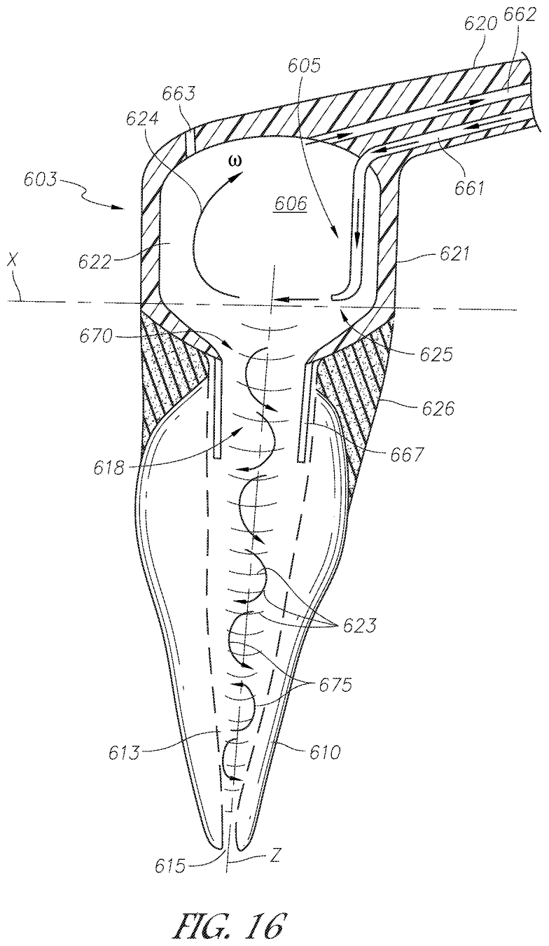

FIG. 16 is a schematic diagram of a coupling member 603 having a fluid motion generator 605 configured to generate a fluid motion 624 in a chamber 606 of the coupling member 603 and/or pressure waves 623 in the fluid 622. As above, the coupling member 603 can be formed with or coupled to a distal end portion 621 of a handpiece 620. In the embodiment of FIG. 16, the coupling member 603 is coupled to the tooth 610 by way of a tooth seal 626. In some embodiments, the coupling member 603 can be pressed against the tooth 610 during a treatment procedure. A mating tube 667 can be used to align and/or secure the positioning member 603 to the tooth 610. The mating tube 667 can extend about and define an access aperture or port 670 of the coupling member 603 that provides fluid communication between the chamber 606 and the tooth 610. As shown in FIG. 16, the root canal 613 can have a central axis Z extending along a major length or dimension of the root canal 613. The mating tube 667 can prevent the coupling member 603 from translating in a direction X transverse to the central axis Z.

In the embodiment of FIG. 16, the fluid motion generator 605 can be disposed in the chamber 606, which can be outside the tooth 610. The pressure wave generator 605 can be positioned offset from a central region of the coupling member 603, e.g., positioned along a wall of the coupling member 603. As shown in FIG. 16, the pressure wave generator 605 can comprise a fluid inlet 661 configured to supply a fluid 622 to the chamber 606 of the coupling member 603. For example, in some embodiments, the fluid inlet 661 can be configured to supply a treatment fluid. In embodiments using a treatment fluid, the fluid motion generator 605 can be configured to clean the tooth. As explained herein, the fluid motion generator 605 can enhance the effects of the treatment fluid to clean both larger canal spaces and smaller cracks and crevices of the tooth. The fluid motion generator 605 can be activated to generate a broad spectrum of acoustic frequencies to clean different types of material from differently-sized spaces of the tooth 610.

As shown in FIG. 16, the fluid inlet 661 can supply the fluid 622 into the chamber 606 to introduce fluid motion 624 in the chamber. As shown in FIG. 16, for example, the fluid motion 624 comprises a rotational flow of fluid in the chamber 606. The fluid motion 624 can define a rotational flow path or field substantially about an axis transverse to the central axis Z of the root canal 613 (e.g. the fluid flows in a direction w about axes transverse to the central axis Z).

For example, one way to induce the fluid motion 624 illustrated in FIG. 16 is to position a distal end portion 625 of the fluid motion generator 605 adjacent to the access port 670 of the coupling member 603. The distal end portion 625 of the fluid inlet 661 can direct a stream or beam of fluid across the access opening 618 of the tooth 610 (and/or across the access port 670 of the coupling member 603 and chamber 606) along an X direction transverse to the central axis Z of the root canal 613. For example, in some embodiments, the fluid inlet 661 can direct a stream of fluid 622 along a direction that is substantially perpendicular to the central axis Z of the root canal 613, e.g., in a direction that is more or less orthogonal to a major axis of the canal 613. Furthermore, the fluid (e.g., the momentum of the fluid stream) can be directed along and/or substantially parallel to a plane near the proximal-most end of the access port 670 to induce the fluid motion 624 shown in FIG. 16. The fluid flow 624 across the access port 670 can be varied to control a desired apical pressure near the apex of the tooth 610. For example, the momentum of the fluid motion 624 can be controllably adjusted by way of the system controller. Further, the angle relative to the central axis Z can also be adjusted to control apical pressure. Indeed, the parameters of the pressure wave generator 605 can be adjusted to increase, decrease, and/or maintain the apical pressure to improve patient outcomes.

The motion 624 of the fluid 622 in the chamber 606 across the port 670 (which may induce flow in the rotational direction w shown in FIG. 16) can induce vortices 675 throughout the root canal 613. For example, shear forces in the fluid 622 induced by the rotational flow 624 can generate vortices 675 that rotate or circulate in opposite directions (e.g., clockwise and counterclockwise as shown in FIG. 16). For example, in some arrangements, stronger vortices 675 may be created near the access port 670, and weaker vortices 675 may be created nearer the apical opening 615. In some embodiments, the vortices 675 may gradually weaken along the length of the canal from a point near the access port 70 to the apical opening 615. The weaker vortices 675 nearer the apical opening 615 may help to prevent or reduce the risk of extrusion of material through the apex of the tooth, which can lead to safer treatment procedures. The vortices 675 can be adjusted by controlling the parameters of the pressure wave generator 605 and the fluid motion 624 generated by the pressure wave generator 605. In some embodiments, the vortices 675 can be steady in size, shape, and/or direction. In other embodiments, the vortices 675 can be unsteady or chaotic.

Furthermore, the alternating directions of the vortices along the root canal 613 can advantageously create a negative pressure (or low positive pressure) near the apical opening 615 of the tooth 610. For example, the vortices 675, which also rotate about axes transverse to the central axis Z of the root canal 613, may cause micro-flows upwards towards the access opening 618 such that fluid 622 tends to experience a slightly negative pressure (e.g., a slight tendency to flow upwards through the canal 613 towards the access opening 618) near the apical opening 615. In some embodiments, the negative pressure near the apical opening 615 can prevent material in the tooth 610 from extruding out through the apical opening 615. In other treatments, for example, the pressure near the apical opening 615 can be positive such that material is pushed out, or extruded, through the apical opening 615 and into the jaw of the patient. Such extrusion can lead to undesirable patient outcomes such as infection, high levels of pain, etc.

In some embodiments, it can be advantageous to dispose the fluid motion generator 605 within the chamber 606 and to use a coupling member 603 with an access port 670 as large as possible. By increasing the diameter or major dimension of the access port 670, more energy can be directed into the tooth 610, which can enhance the tooth cleaning procedure. Increasing the diameter or major dimension can also enhance the obturation procedure when used in such embodiments. In some embodiments, for example, the mating tube 667 may not be used so as to increase the size of the access port 670 by about twice a thickness of the walls of the mating tube 667. Accordingly, in various embodiments, the access port 670 of the coupling member 603 can be at least as large as a diameter or major dimension of the access opening 618 formed in the tooth 610. In some embodiments, for example, the access port 670 can be about the same size as the access opening 618 formed in the tooth 610.

In some embodiments, the fluid motion generator 605 can also be configured to generate pressure waves 623 through the fluid 622 and the tooth 610. In some embodiments, in cleaning treatments, a combination of the pressure waves 623, fluid motion 624, and chemistry of the treatment fluid can act to substantially remove unhealthy materials from the tooth 610, including in small spaces, cracks, and crevices of the tooth 610. Waste fluid and detached materials can be removed from the tooth 610 and/or the chamber 606 by way of a fluid outlet 662. In some embodiments, one or more vents 663 can be provided through the coupling member 603 to regulate the pressure in the chamber 606. In filling or obturation procedures, the pressure waves 623, fluid motion 624, and chemistry of the obturation material can act to substantially fill the entire root canal system.

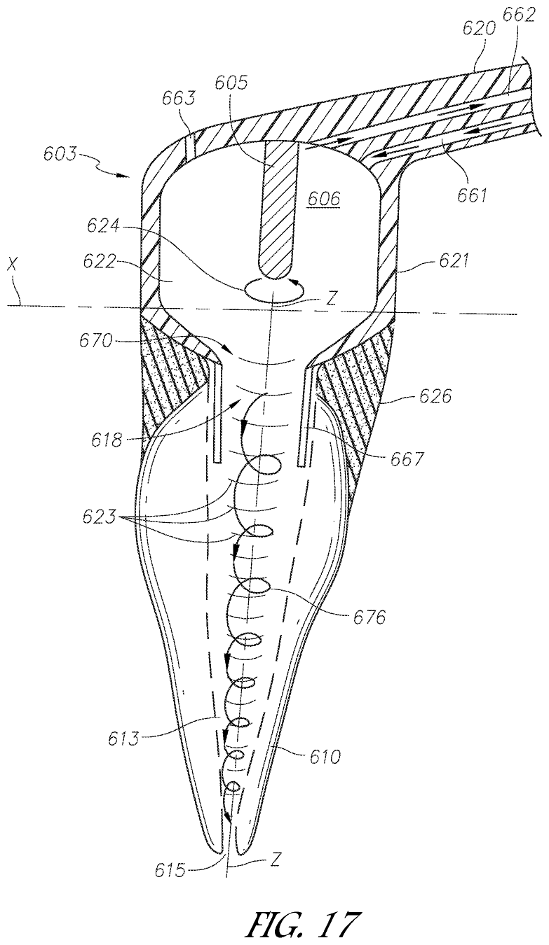

FIG. 17 is a schematic diagram of a coupling member 603 having a fluid motion generator 605 substantially aligned with a central axis Z of the root canal 613. The fluid motion generator 605 can be any suitable fluid motion generator disclosed herein, such as a liquid jet device, a fluid inlet, a light emitting element, etc. For example, in some embodiments the fluid motion generator 605 may include a sonic, ultrasonic, or megasonic device (e.g., a sonic, ultrasonic, or megasonic paddle, horn, or piezoelectric transducer), a mechanical stirrer (e.g., a motorized propeller or paddle or rotating/vibrating/pulsating disk or cylinder), an optical system that can provides optical energy to the chamber 606 (e.g., an optical fiber that propagates laser light into the chamber 606), or any other device that can cause sufficient rotational fluid motion and/or acoustic waves to be generated in the tooth or in a propagation medium in the tooth (e.g., the fluid retained in a tooth chamber). For example, in some embodiments, the fluid motion generator 605 of FIG. 17 can be a nozzle configured to output a fluid 622 into the chamber 606. Unlike the embodiment of FIG. 16, which may be offset from the Z-axis, the fluid motion generator 605 of FIG. 17 is generally aligned with the Z-axis.

Furthermore, unlike the embodiment of FIG. 16, the fluid motion generator 605 of FIG. 17 can be configured to generate a fluid motion 624 of flowable material 622 about the Z-axis. The fluid motion 624 substantially around the Z-axis can generate a swirl 676 of fluid, which can propagate through the root canal 623. Similar to the embodiments above, the rotational power of the fluid motion 624 can be adjusted to control the amount of swirl 676 to assist with the treatment procedure. As above, in some embodiments, the fluid motion generator 605 can also be configured to generate pressure waves 623 through the fluid 622 and the tooth 610. In cleaning treatments, the fluid motion generator 605 can clean substantially the entire root canal 613. In obturation treatments, the pressure wave generator 605 can fill or obturate substantially the entire root canal 613, including branch structures.

Additional examples and details of fluid motion generators and pressure wave generators can be found in, for example, FIGS. 6, 12A-16B, and 20A-23 and the associated disclosure of U.S. Pat. No. 9,492,244, issued Nov. 15, 2016; paragraphs [0088]-[0095], FIGS. 1A-2B, and 5-10G and the associated disclosure of U.S. Patent Publication No. 2014/0220505, published Aug. 7, 2014; column 17, line 60 to column 18, line 49 of the U.S. Pat. No. 9,877,801, issued Jan. 30, 2018, and in FIGS. 15-24D and the associated disclosure of U.S. Pat. No. 10,363,120, issued Jul. 30, 2019, each of which is incorporated by reference herein in its entirety and for all purposes.

### Examples of Power Generated by Pressure Wave Generators

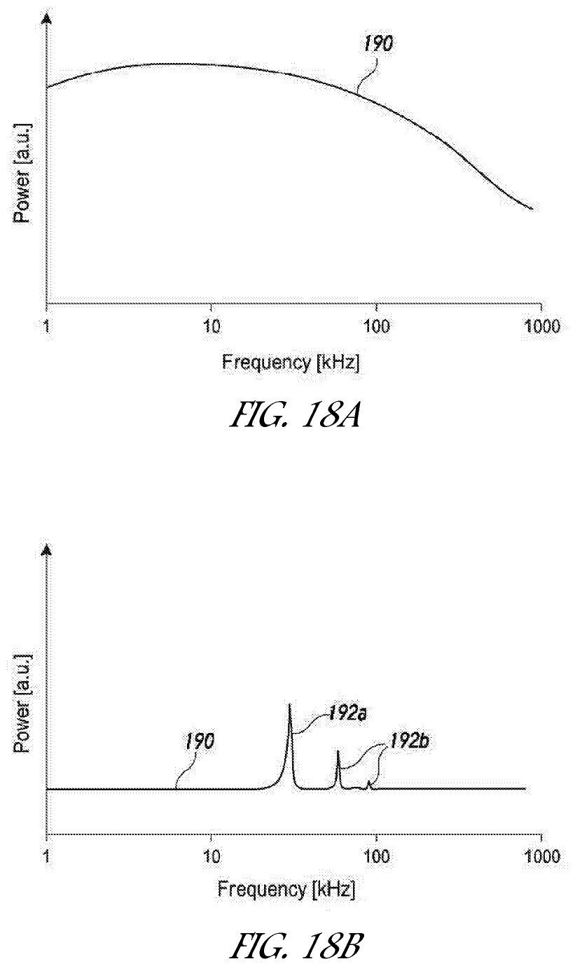

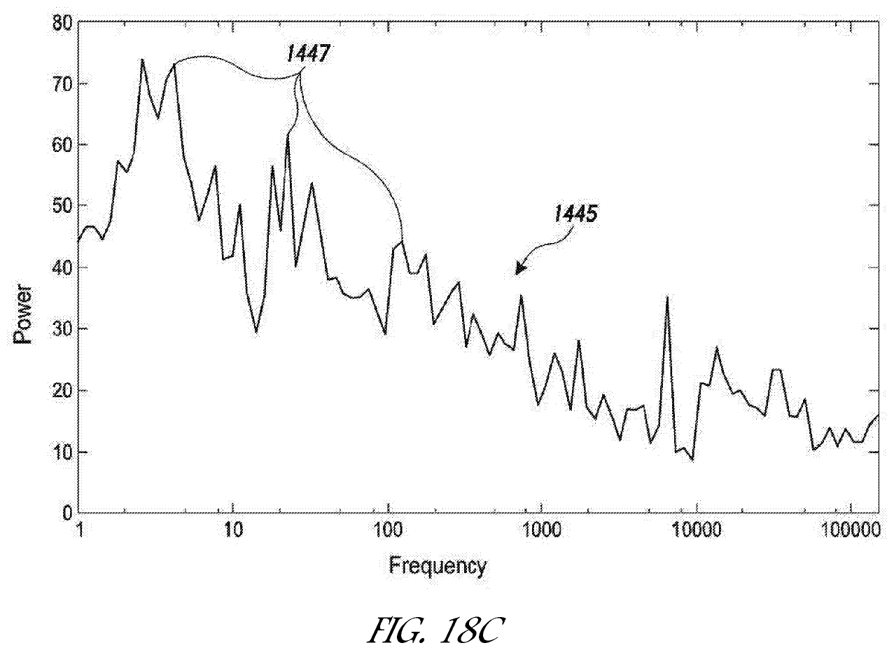

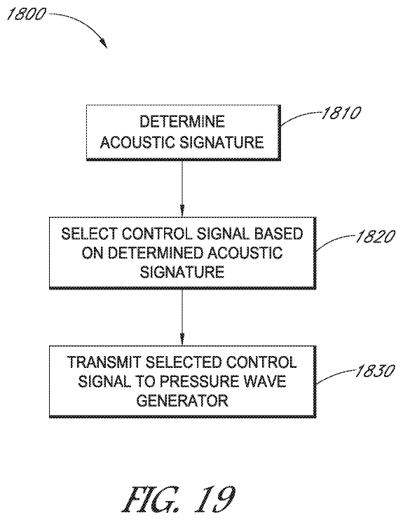

FIGS. 18A and 18B are graphs that schematically illustrate possible examples of power that can be generated by different embodiments of pressure wave generators described herein. These graphs schematically show acoustic power (in arbitrary units) on the vertical axis as a function of acoustic frequency (in kHz) on the horizontal axis. The acoustic power in the tooth may influence, cause, or increase the strength of effects including, e.g., acoustic cavitation (e.g., cavitation bubble formation and collapse, microjet formation), acoustic streaming, microerosion, fluid agitation, fluid circulation and/or rotational motion, sonoporation, sonochemistry, and so forth, which may act to dissociate organic material in or on the tooth and effectively clean the undesirable materials, e.g., undesirable organic and/or inorganic materials and deposits. In some embodiments, these effects can enhance or enable the obturation or filling of treated root canals or other treatment regions of the tooth. For example, the embodiments disclosed herein can advantageously obturate or fill substantially the entire canal(s) and/or branch structures therefrom, as explained in greater detail above. In various embodiments, the pressure wave generator can produce a pressure wave including acoustic power (at least) at frequencies above: about 1 Hz, about 0.5 kHz, about 1 kHz, about 10 kHz, about 20 kHz, about 50 kHz, about 100 kHz, or greater. The pressure wave can have acoustic power at other frequencies as well (e.g., at frequencies below the aforelisted frequencies).

The graph in FIG. 18A represents a schematic example of acoustic power generated by a liquid jet impacting a surface disposed within a chamber on or around the tooth that is substantially filled with liquid and by the interaction of the liquid jet with fluid in the chamber. This schematic example shows a broadband spectrum 190 of acoustic power with significant power extending from about 1 Hz to about 1000 kHz, including, e.g., significant power in a range of about 1 kHz to about 1000 kHz (e.g., the bandwidth can be about 1000 kHz). The bandwidth of the acoustic energy spectrum may, in some cases, be measured in terms of the 3-decibel (3-dB) bandwidth (e.g., the full-width at half-maximum or FWHM of the acoustic power spectrum). In various examples, a broadband acoustic power spectrum can include significant power in a bandwidth in a range from about 1 Hz to about 500 kHz, in a range from about 1 kHz to about 500 kHz, in a range from about 10 kHz to about 100 kHz, or some other range of frequencies. In some implementations, a broadband spectrum can include acoustic power above about 1 MHz. In some embodiments, the pressure wave generator can produce broadband acoustic power with peak power at about 10 kHz and a bandwidth of about 100 kHz. In various embodiments, the bandwidth of a broadband acoustic power spectrum is greater than about 10 kHz, greater than about 50 kHz, greater than about 100 kHz, greater than about 250 kHz, greater than about 500 kHz, greater than about 1 MHz, or some other value. In some cleaning methods, acoustic power between about 1 Hz and about 200 kHz, e.g., in a range of about 20 kHz to about 200 kHz may be particularly effective at cleaning teeth. The acoustic power can have substantial power at frequencies greater than about 1 kHz, greater than about 10 kHz, greater than about 100 kHz, or greater than about 500 kHz. Substantial power can include, for example, an amount of power that is greater than 10%, greater than 25%, greater than 35%, or greater than 50% of the total acoustic power (e.g., the acoustic power integrated over all frequencies). In some arrangements, the broadband spectrum 190 can include one or more peaks, e.g., peaks in the audible, ultrasonic, and/or megasonic frequency ranges.

The graph in FIG. 18B represents a schematic example of acoustic power generated by an ultrasonic transducer disposed in a chamber on or around the tooth that is substantially filled with liquid. This schematic example shows a relatively narrowband spectrum 192 of acoustic power with a highest peak 192a near the fundamental frequency of about 30 kHz and also shows peaks 192b near the first few harmonic frequencies. The bandwidth of the acoustic power near the peak may be about 5 to 10 kHz, and can be seen to be much narrower than the bandwidth of the acoustic power schematically illustrated in FIG. 18A. In other embodiments, the bandwidth of the acoustic power can be about 1 kHz, about 5 kHz, about 10 kHz, about 20 kHz, about 50 kHz, about 100 kHz, or some other value. The acoustic power of the example spectrum 192 has most of its power at the fundamental frequency and first few harmonics, and therefore the ultrasonic transducer of this example may provide acoustic power at a relatively narrow range of frequencies (e.g., near the fundamental and harmonic frequencies). The acoustic power of the example spectrum 190 exhibits relatively broadband power (with a relatively high bandwidth compared to the spectrum 192), and the example liquid jet can provide acoustic power at significantly more frequencies than the example ultrasonic transducer. For example, the relatively broadband power of the example spectrum 190 illustrates that the example jet device provides acoustic power at these multiple frequencies with energy sufficient to break the bonds between the decayed and healthy material so as to substantially remove the decayed material from the carious region.

It is believed, although not required, that pressure waves having broadband acoustic power (see, e.g., the example shown in FIG. 18A) can generate acoustic cavitation or other means of cleaning and disinfection that is more effective at cleaning teeth (including cleaning, e.g., unhealthy materials in or on the tooth) than cavitation generated by pressure waves having a narrowband acoustic power spectrum (see, e.g., the example shown in FIG. 18B). Further, broadband acoustic power can also generate sufficient energy at frequencies capable of obturating or filling a root canal or other treatment region (such as a treated carious region on an exterior surface of the tooth). For example, a broadband spectrum of acoustic power can produce a relatively broad range of bubble sizes in the cavitation cloud and on the surfaces on the tooth, and the implosion of these bubbles may be more effective at disrupting tissue than bubbles having a narrow size range. Relatively broadband acoustic power may also allow acoustic energy to work on a range of length scales, e.g., from the cellular scale up to the tissue scale. Accordingly, pressure wave generators that produce a broadband acoustic power spectrum (e.g., some embodiments of a liquid jet) can be more effective at tooth cleaning for some treatments than pressure wave generators that produce a narrowband acoustic power spectrum. In some embodiments, multiple narrowband pressure wave generators can be used to produce a relatively broad range of acoustic power. For example, multiple ultrasonic tips, each tuned to produce acoustic power at a different peak frequency, can be used. As used herein, broadband frequencies and broadband frequency spectrum is defined regardless of secondary effects such as harmonics of the main frequencies and regardless of any noise introduced by measurement or data processing (e.g., FFT); that is, these terms should be understood when only considering all main frequencies activated by the pressure wave generator.

FIG. 18C is a graph of an acoustic power spectrum 1445 generated at multiple frequencies by the pressure wave generators disclosed herein. For example, the spectrum 1445 in FIG. 18C is an example of acoustic power generated by a liquid jet impacting a surface disposed within a chamber on, in, or around the tooth that is substantially filled with liquid and by the interaction of the liquid jet with fluid in the chamber. The spectrum 1445 of FIG. 18C represents acoustic power detected by a sensor spaced apart from the source of the acoustic energy, e.g., the pressure wave generator. The data was acquired inside an insulated water tank data when the distance between the power wave generator and the hydrophone (e.g., sensor) being about 8 inches. The vertical axis of the plot represents a measure of acoustic power: Log (P_{acoustic}²), referred to herein as “power units”. The units of P_{acoustic} in the measurement were μPa (micro Pascal). Thus, it should be appreciated that the actual power at the source may be of a different magnitude because the sensor is spaced from the acoustic power generator. However, the general profile of the power spectrum at the source should be the same as the spectrum 1445 detected at the sensor and plotted in FIG. 18C. It should also be understood that, although the plot shows frequencies only up to 100 KHz, the power above 100 KHz was greater than zero—the data just was not plotted. It should further be noted that, as would be appreciated by one skilled in the art, the plot and the values would also depend on other parameters, such as, for example, the size and shape of the tank in which data was acquired, the insulation of the inner surface of the tank, the relative distance between the source (e.g., power wave generator), and the free water surface of the tank.

As shown in FIG. 18C, the spectrum 1445 can include acoustic power at multiple frequencies 1447, e.g., multiple discrete frequencies. In particular, the spectrum 1445 illustrated in FIG. 18C includes acoustic power at frequencies in a range of about 1 Hz to about 100 KHz. The acoustic power can be in a range of about 10 power units to about 80 power units at these frequencies. In some arrangements, the acoustic power can be in a range of about 30 power units to about 75 power units at frequencies in a range of about 1 Hz to about 10 kHz. In some arrangements, the acoustic power can be in a range of about 10 power units to about 30 power units at frequencies in a range of about 1 KHz to about 100 kHz. In some embodiments, for example, the broadband frequency range of the pressure waves generated by the pressure wave generators disclosed herein can comprise a substantially white noise distribution of frequencies.

Pressure wave generators that generate acoustic power associated with the spectrum 1445 of FIG. 18C can advantageously and surprisingly clean undesirable materials from teeth. As explained above, the generation of power at multiple frequencies can help to remove various types of organic and/or inorganic materials that have different material or physical characteristics, and/or different bonding strengths at various frequencies. For example, some undesirable materials may be removed from the teeth and/or gums at relatively low acoustic frequencies, while other materials may be removed from the teeth at relatively high acoustic frequencies, while still other materials may be removed at intermediate frequencies between the relatively low and relatively high frequencies. As shown in FIG. 18C, lower frequency cleaning phases can be activated at higher powers, and higher frequency cleaning phases can be activated at lower powers. In other embodiments, low frequency cleaning phases may be activated at relatively low powers, and high frequency cleaning phases may be activated at relatively high powers. Pressure wave generators that generate acoustic power at multiple frequencies (e.g., multiple discrete frequencies) are capable of cleaning undesirable materials and decayed matter from interior and/or exterior portions of teeth.

In the embodiments disclosed herein, treatment procedures can be activated to generate acoustic power at various frequency ranges. For example, some treatment phases may be activated at lower frequencies, and other treatment phases may be activated at higher frequencies. The pressure wave generators disclosed herein can be adapted to controllably generate acoustic power at any suitable frequencies 1447 of the spectrum 1445. For example, the pressure wave generators disclosed herein can be adapted to generate power at multiple frequencies 1447 simultaneously, e.g., such that the delivered acoustic power in a particular treatment procedure can include a desired combination of individual frequencies. For example, in some procedures, power may be generated across the entire frequency spectrum 1445. In some treatment phases, the pressure wave generator can deliver acoustic power at only relatively low frequencies, and in other treatment phases, the pressure wave generator can deliver power at only relatively high frequencies, as explained herein. Further, depending on the desired treatment procedure, the pressure wave generator can automatically or manually transition between frequencies 1447 according to a desired pattern, or can transition between frequencies 1447 randomly. In some arrangements, relatively low frequencies can be associated with large-scale bulk fluid movement, and relatively high frequencies can be associated with small-scale, high-energy oscillations.

In some embodiments, the treatment procedure may include one or more treatment phases. In each treatment phase, energy can be applied at a different frequency or band of frequencies. For example, in one phase, energy (e.g., pressure or acoustic waves) propagating at a relatively low frequency (or band of frequencies) may be generated. The low frequency pressure waves can interact with the treatment fluid in the chamber and can induce removal of large-scale dental deposits or materials. Without being limited by theory, the low frequency pressure waves can remove a substantial portion of the unhealthy materials in the tooth. For example, the low frequency waves may have a sufficiently high energy at suitably low frequencies to remove large deposits or materials from the tooth. The acoustic power at the relatively low frequencies can include acoustic power at any suitable low-frequency band of the power spectrum of the pressure wave generator (see, e.g., FIG. 18C). For example, in some embodiments, the acoustic power in the first, low-frequency range can include one or more frequencies in a range of about 0.1 Hz to about 100 Hz, for example in a range of about 1 Hz to about 50 Hz in some arrangements.

In another phase, acoustic energy may be generated at relatively high frequencies. At higher frequencies, the pressure wave generator can be configured to remove smaller deposits and debris. For example, at higher frequencies, the pressure waves can propagate through the treatment fluid. The higher frequency waves can remove smaller portions from relatively small locations, such as crevices, cracks, spaces, and irregular surfaces of the tooth. In some embodiments, degassed liquid can be used to enhance the removal of matter from these small spaces. When the higher frequency cleaning is performed after the lower frequency cleaning, in some embodiments, the high frequency waves (and/or intermediate frequency waves) can clean the remainder of the unhealthy material left behind from the low frequency cleaning. In the relatively high frequency phases, acoustic energy can be generated in a range of about 10 kHz to about 1000 kHz, e.g., in a range of about 100 kHz to about 500 kHz.

In some embodiments, the treatment procedure can progress from the relatively low frequencies (or bands of frequencies) toward higher frequencies (or bands of frequencies). For example, the procedure can move from the relatively low frequency phase(s), through intermediate frequency phase(s), until the high frequency phase(s) are reached. Thus, in some embodiments, the treatment procedure can provide a gradual and/or substantially continuous transition between relatively low and relatively high frequencies. As the treatment progresses through the frequencies, unhealthy dental deposits or materials of varying size and type can be removed by the pressure wave generator. In other embodiments, however, the treatment procedure can transition or switch between frequencies (or bands of frequencies) or phases (e.g., between high, low and/or intermediate frequencies or bands of frequencies) at discrete levels. At various intermediate frequency ranges, acoustic energy can be generated in a range of about 100 Hz to about 10 kHz. For example, in some embodiments, the various phases of the treatment procedures described above may be activated by the user or clinician, or the pressure wave generator can be configured to automatically transition between the phases. In some embodiments, for example, the pressure wave generator can randomly switch between high, low, and intermediate frequencies.

Various treatment procedures may include any suitable number of treatment phases at various different frequencies. Furthermore, although various low- and high-frequency phases may be described above as occurring in a particular order, in other embodiments, the order of activating the low- and high-frequency phases, and/or any intermediate frequency phases, may be any suitable order. Furthermore, the treatment procedures and phases described herein can also be used to fill or obturate treatment regions of a tooth after cleaning. In obturation procedures, the embodiments disclosed herein can advantageously obturate or fill substantially the entire canal(s) and/or branch structures therefrom, as explained in greater detail herein

FIG. 19 depicts an embodiment of a process 1800 for treating a treatment region of a tooth. In some embodiments, the process 1800 can include a step 1810 of determining an acoustic signature for acoustic waves in the treatment region of the tooth sufficient for treating (e.g., cleaning, obturating, etc.) the treatment region of the tooth. In some embodiments, the process 1800 can include imaging the tooth. Imaging of the tooth can be performed using any suitable image system or apparatus (cone beam CT, for example). In some embodiments, determining an acoustic signature for acoustic waves in the treatment region of the tooth can be based on information detected from imaging the tooth. For example, frequencies used to clean or fill a treatment region can be determined based on, for example, the shape and size of the tooth, the shape and size of the treatment region, and other exterior and internal structure of the tooth (for example, cracks, the canal spaces, dentinal tubules, etc.). In some embodiments, the acoustic signature can be determined based on these frequencies.

In some embodiments, the determining an acoustic signature can be performed using a controller or control system, such as controller 420, or any other suitable computing/processing system or device.

After the acoustic signature is determined, the process 1800 can move to a step 1820 in which a control signal is selected based on the determinedacoustic signature determined in step 1810. The control signal can be selected to cause the generation of the determinedacoustic signature within the treatment region of the tooth. The selection of the control signal may be based at least in part on the specifications of a pressure wave generator or pressure wave generators to which the control signal is to be provided.

In some embodiments, selection of the control signal can be performed using a controller or control system, such as controller 420, or any other suitable computing/processing system or device.

After selection of the control signal, the process 1800 can move to a step 1830 in which the selected control signal is transmitted to one or more pressure wave generators, such as pressure wave generator 410, to cause the pressure wave generator(s) 410 to produce pressure waves in the treatment fluid. The control signals can cause the pressure wave generator(s) 410 to produce pressure waves based on the determined acoustic signature. If properly selected, the control signal can cause the pressure wave generator(s) 410 to produce pressure waves at the determined acoustic signature.

In some instances, for example, due to changes in the treatment region during a treatment procedure or due to an improperly selected control signal, a control signal may cause the pressure wave generator(s) 410 to generate pressure waves having an acoustic signature different from the determined acoustic signature. In such instances, it may be desirable to adjust the control signals so as to change the acoustic signature of the pressure waves produced by the pressure wave generator(s) 410. In some embodiments, methods can include steps for measuring acoustic properties in a treatment region and adjusting the control signals provided to the pressure wave generator(s). For example, FIG. 20 depicts an embodiment of a process 1900 for treating a treatment region of a tooth.

As shown in FIG. 20, the process 1900 can include steps 1910, 1920, and 1930, that can be generally the same or similar to steps 1810, 1820, and 1830 of process 1800. For example, the process 1900 includes a step 1910 of determining an acoustic signature for acoustic waves in the treatment region of the tooth sufficient for treating (e.g., cleaning, obturating, etc.) the treatment region of the tooth. In some embodiments, the process 1900 can include imaging the tooth. Imaging of the tooth can be performed using any suitable image system or apparatus (cone beam CT, for example). In some embodiments, determining an acoustic signature for acoustic waves in the treatment region of the tooth can be based on information detected from imaging the tooth. For example, frequencies used to clean or fill a treatment region can be determined based on, for example, the shape and size of the tooth, the shape and size of the treatment region, and other exterior and internal structure of the tooth (for example, cracks, the canal spaces, dentinal tubules, etc.). In some embodiments, the acoustic signature can be determined based on these frequencies. In some embodiments, determining an acoustic signature can be performed using a controller or control system, such as controller 420, or any other suitable computing/processing system or device.

Once the acoustic signature is determined, the process 1900 can move to a step 1920 in which a control signal is selected based on the determined acoustic signature determined in step 1910. The control signal can be selected to cause the generation of the determined acoustic signature within the treatment region of the tooth. The selection of the control signal may be based at least in part on the specifications of a pressure wave generator or pressure wave generators to which the control signal is to be provided.

In some embodiments, selection of the control signal can be performed using a controller or control system, such as controller 420, or any other suitable computing/processing system or device.

After selection of the control signal, the process 1900 can move to a step 1930 in which the selected control signal is transmitted to one or more pressure wave generators, such as pressure wave generator 410, to cause the pressure wave generator(s) 410 to produce pressure waves in the treatment fluid. The control signals can cause the pressure wave generator(s) 410 to produce pressure waves based on the determined acoustic signature. If properly selected, the control signal can cause the pressure wave generator(s) 410 to produce pressure waves at the determined acoustic signature.

The process 1900 can include a step 1940 of measuring the acoustic properties of the treatment fluid generated within the treatment region. Measurement of acoustic properties can be performed using any suitable sensor or sensor system. In some embodiments, the sensor or sensor system can communicate the measured acoustic properties to a controller or control system, such as controller 420, or any other suitable computing/processing system or device.

The process 1900 can include a step 1950 in which it is determined if the measured acoustic properties match the determiend acoustic signature. The determination may be performed by a controller or control system, such as controller 420, or any other suitable computing/processing system or device. If it is determined that the measured acoustic properties match the determined acoustic signature, the process 1900 can return to the step 1940, and the acoustic properties can continue to be monitored.

If it is determined that the acoustic properties do not match the determined acoustic signature, the process 1900 can move to a step 1960 in which the control signal is adjusted to change the acoustic signature of the pressure waves generated within the treatment region. The control signal can be adjusted to match or try to match the acoustic signature of the pressure waves generated within the treatment region to the determined acoustic signature. Adjustments to the control signal can be determined based on the determined acoustic signature and differences between the measured acoustic properties and the determined acoustic signature. After the control signature is adjusted, the process 1900 can return to the step 1940, and the acoustic properties can continue to be monitored.

Beneficially, the embodiments disclosed herein enable the clinician to tailor a treatment procedure to the patient's anatomy. For example, as explained herein, the acoustic signature can be determined based on the patient's unique tooth structure so as to adequately clean or fill the treatment region of that specific tooth. The predetermined acoustic signature can comprise acoustic frequencies across a wide band of frequencies, in some cases. In some arrangements, the predetermined acoustic signature can include higher energy levels at a certain frequency or frequencies, for example, to clean or fill a particular region of the treatment region.

### Degassed Treatment Fluids

As will be described below, the treatment fluid described herein (and/or any of solutions added to the treatment fluid) can be degassed compared to normal liquids used in dental offices. For example, degassed distilled water can be used (with or without the addition of chemical agents or solutes).

### Examples of Possible Effects of Dissolved Gases in the Treatment Fluid

In some procedures, the treatment fluid can include dissolved gases (e.g., air). For example, the fluids used in dental offices generally have a normal dissolved gas content (e.g., determined from the temperature and pressure of the fluid based on Henry's law). During cleaning procedures using a pressure wave generator, the acoustic field of the pressure wave generator and/or the flow or circulation of fluids in the chamber can cause some of the dissolved gas to come out of solution and form bubbles.

The bubbles can block small passageways or cracks or surface irregularities in the tooth, and such blockages can act as if there were a “vapor lock” in the small passageways. In some such procedures, the presence of bubbles may at least partially block, impede, or redirect propagation of acoustic waves past the bubbles and may at least partially inhibit or prevent cleaning action from reaching, for example, unhealthy dental materials in tubules and small spaces of the tooth. The bubbles may block fluid flow or circulation from reaching these difficult-to-reach, or otherwise small, regions, which may prevent or inhibit a treatment solution from reaching these areas of the tooth.

In certain procedures, cavitation is believed to play a role in cleaning the tooth. Without wishing to be bound by any particular theory, the physical process of cavitation inception may be, in some ways, similar to boiling. One possible difference between cavitation and boiling is the thermodynamic paths that precede the formation of the vapor in the fluid. Boiling can occur when the local vapor pressure of the liquid rises above the local ambient pressure in the liquid, and sufficient energy is present to cause the phase change from liquid to a gas. It is believed that cavitation inception can occur when the local ambient pressure in the liquid decreases sufficiently below the saturated vapor pressure, which has a value given in part by the tensile strength of the liquid at the local temperature. Therefore, it is believed, although not required, that cavitation inception is not determined by the vapor pressure, but instead by the pressure of the largest nuclei, or by the difference between the vapor pressure and the pressure of the largest nuclei. As such, it is believed that subjecting a fluid to a pressure slightly lower than the vapor pressure generally does not cause cavitation inception. However, the solubility of a gas in a liquid is proportional to pressure; therefore lowering the pressure may tend to cause some of the dissolved gas inside the fluid to be released in the form of gas bubbles that are relatively large compared to the size of bubbles formed at cavitation inception. These relatively large gas bubbles may be misinterpreted as being vapor cavitation bubbles, and their presence in a fluid may have been mistakenly described in certain reports in the literature as being caused by cavitation, when cavitation may not have been present.

In the last stage of collapse of vapor cavitation bubbles, the velocity of the bubble wall may even exceed the speed of sound and create strong shock waves inside the fluid. The vapor cavitation bubble may also contain some amount of gas, which may act as a buffer and slow down the rate of collapse and reduce the intensity of the shockwaves. Therefore, in certain procedures that utilize cavitation bubbles for tooth cleaning, it may be advantageous to reduce the amount of the dissolved air in the fluid to prevent such losses.

The presence of bubbles that have come out of solution from the treatment fluid may lead to other disadvantages during certain procedures. For example, if the pressure wave generator produces cavitation, the agitation (e.g. pressure drop) used to induce the cavitation may cause the release of the dissolved air content before the water molecules have a chance to form a cavitation bubble. The already-formed gas bubble may act as a nucleation site for the water molecules during the phase change (which was intended to form a cavitation bubble). When the agitation is over, the cavitation bubble is expected to collapse and create pressure waves. However, cavitation bubble collapse might happen with reduced efficiency, because the gas-filled bubble may not collapse and may instead remain as a bubble. Thus, the presence of gas in the treatment fluid may reduce the effectiveness of the cavitation process as many of the cavitation bubbles may be wasted by merging with gas-filled bubbles. Additionally, bubbles in the fluid may act as a cushion to damp pressure waves propagating in the region of the fluid comprising the bubbles, which may disrupt effective propagation of the pressure waves past the bubbles. Some bubbles may either form on or between tooth surfaces, or be transferred there by the flow or circulation of fluid in the tooth. The bubbles may be hard to remove due to relatively high surface tension forces. This may result in blocking the transfer of chemicals and/or pressure waves into the irregular surfaces and small spaces in and between teeth, and therefore may disrupt or reduce the efficacy of the treatment.

### Examples of Degassed Treatment Fluids

Accordingly, it may be advantageous in some systems and methods to use a degassed fluid, which can inhibit, reduce, or prevent bubbles from coming out of solution during treatments as compared to systems and methods that use normal (e.g., non-degassed) fluids. In dental procedures in which the treatment fluid has a reduced gas content (compared with the normal fluids) tooth surfaces or tiny spaces in the tooth may be free of bubbles that have come out of solution. Acoustic waves generated by the pressure wave generator can propagate through the degassed fluid to reach and clean the surfaces, cracks, and tooth spaces and cavities. In some procedures, the degassed fluid can be able to penetrate spaces as small as about 500 microns, 200 microns, 100 microns, 10 microns, 5 microns, 1 micron, or smaller, because the degassed fluid is sufficiently gas-free that bubbles are inhibited from coming out of solution and blocking these spaces (as compared to use of fluids with normal dissolved gas content).

For example, in some systems and methods, the degassed fluid can have a dissolved gas content that is reduced when compared to the “normal” gas content of water. For example, according to Henry's law, the “normal” amount of dissolved air in water (at 25 C and 1 atmosphere) is about 23 mg/L, which includes about 9 mg/L of dissolved oxygen and about 14 mg/L of dissolved nitrogen. In some embodiments, the degassed fluid has a dissolved gas content that is reduced to approximately 10%-40% of its “normal” amount as delivered from a source of fluid (e.g., before degassing). In other embodiments, the dissolved gas content of the degassed fluid can be reduced to approximately 5%-50% or 1%-70% of the normal gas content of the fluid. In some treatments, the dissolved gas content can be less than about 70%, less than about 50%, less than about 40%, less than about 30%, less than about 20%, less than about 10%, less than about 5%, or less than about 1% of the normal gas amount.

In some embodiments, the amount of dissolved gas in the degassed fluid can be measured in terms of the amount of dissolved oxygen (rather than the amount of dissolved air), because the amount of dissolved oxygen can be more readily measured (e.g., via titration or optical or electrochemical sensors) than the amount of dissolved air in the fluid. Thus, a measurement of dissolved oxygen in the fluid can serve as a proxy for the amount of dissolved air in the fluid. In some such embodiments, the amount of dissolved oxygen in the degassed fluid can be in a range from about 1 mg/L to about 3 mg/L, in a range from about 0.5 mg/L to about 7 mg/L, or some other range. The amount of dissolved oxygen in the degassed fluid can be less than about 7 mg/L, less than about 6 mg/L, less than about 5 mg/L, less than about 4 mg/L, less than about 3 mg/L, less than about 2 mg/L, or less than about 1 mg/L.

In some embodiments, the amount of dissolved gas in the degassed fluid can be in a range from about 2 mg/L to about 20 mg/L, in a range from about 1 mg/L to about 12 mg/L, or some other range. The amount of dissolved gas in the degassed fluid can be less than about 20 mg/L, less than about 18 mg/L, less than about 15 mg/L, less than about 12 mg/L, less than about 10 mg/L, less than about 8 mg/L, less than about 6 mg/L, less than about 4 mg/L, or less than about 2 mg/L.

In other embodiments, the amount of dissolved gas can be measured in terms of air or oxygen percentage per unit volume. For example, the amount of dissolved oxygen (or dissolved air) can be less than about 5% by volume, less than about 1% by volume, less than about 0.5% by volume, or less than about 0.1% by volume.

The amount of dissolved gas in a liquid can be measured in terms of a physical property such as, e.g., fluid viscosity or surface tension. For example, degassing water tends to increase its surface tension. The surface tension of non-degassed water is about 72 mN/m at 20° C. In some embodiments, the surface tension of degassed water can be about 1%, 5%, or 10% greater than non-degassed water.

In some treatment methods, one or more secondary fluids can be added to a primary degassed fluid (e.g., an antiseptic solution can be added to degassed distilled water). In some such methods, the secondary solution(s) can be degassed before being added to the primary degassed fluid. In other applications, the primary degassed fluid can be sufficiently degassed such that inclusion of the secondary fluids (which can have normal dissolved gas content) does not increase the gas content of the combined fluids above what is desired for a particular dental treatment.

In various implementations, the treatment fluid can be provided as degassed liquid inside sealed bags or containers. The fluid can be degassed in a separate setup in the operatory before being added to a fluid reservoir. In an example of an “in-line” implementation, the fluid can be degassed as it flows through the system, for example, by passing the fluid through a degassing unit attached along a fluid line (e.g., the fluid inlet). Examples of degassing units that can be used in various embodiments include: a Liqui-Cel® MiniModule® Membrane Contactor (e.g., models 1.7×5.5 or 1.7×8.75) available from Membrana-Charlotte (Charlotte, N.C.); a PermSelect® silicone membrane module (e.g., model PDMSXA-2500) available from MedArray, Inc. (Ann Arbor, Mich.); and a FiberFlo® hollow fiber cartridge filter (0.03 micron absolute) available from Mar Cor Purification (Skippack, Pa.). The degassing can be done using any of the following degassing techniques or combinations of thereof: heating, helium sparging, vacuum degassing, filtering, freeze-pump-thawing, and sonication.

In some embodiments, degassing the fluid can include de-bubbling the fluid to remove any small gas bubbles that form or may be present in the fluid. De-bubbling can be provided by filtering the fluid. In some embodiments, the fluid may not be degassed (e.g., removing gas dissolved at the molecular level), but can be passed through a de-bubbler to remove the small gas bubbles from the fluid.

In some embodiments, a degassing system can include a dissolved gas sensor to determine whether the treatment fluid is sufficiently degassed for a particular treatment. A dissolved gas sensor can be disposed downstream of a mixing system and used to determine whether mixing of solutes has increased the dissolved gas content of the treatment fluid after addition of solutes, if any. A solute source can include a dissolved gas sensor. For example, a dissolved gas sensor can measure the amount of dissolved oxygen in the fluid as a proxy for the total amount of dissolved gas in the fluid, since dissolved oxygen can be measured more readily than dissolved gas (e.g., nitrogen or helium). Dissolved gas content can be inferred from dissolved oxygen content based at least partly on the ratio of oxygen to total gas in air (e.g., oxygen is about 21% of air by volume). Dissolved gas sensors can include electrochemical sensors, optical sensors, or sensors that perform a dissolved gas analysis. Examples of dissolved gas sensors that can be used with embodiments of various systems disclosed herein include a Pro-Oceanus GTD-Pro or HGTD dissolved gas sensor available from Pro-Oceanus Systems Inc. (Nova Scotia, Canada) and a D-Opto dissolved oxygen sensor available from Zebra-Tech Ltd. (Nelson, New Zealand). In some implementations, a sample of the treatment can be obtained and gases in the sample can be extracted using a vacuum unit. The extracted gases can be analyzed using a gas chromatograph to determine dissolved gas content of the fluid (and composition of the gases in some cases).

Accordingly, fluid delivered to the tooth from a fluid inlet and/or the fluid used to generate the jet in a liquid jet device can comprise a degassed fluid that has a dissolved gas content less than normal fluid. The degassed fluid can be used, for example, to generate the high-velocity liquid beam for generating acoustic waves, to substantially fill or irrigate a chamber, to provide a propagation medium for acoustic waves, to inhibit formation of air (or gas) bubbles in the chamber, and/or to provide flow of the degassed fluid into small spaces in the tooth (e.g., cracks, irregular surfaces, tubules, etc.). In embodiments utilizing a liquid jet, use of a degassed fluid can inhibit bubbles from forming in the jet due to the pressure drop at a nozzle orifice where the liquid jet is formed.

Thus, examples of methods for dental and/or endodontic treatment comprise flowing a degassed fluid onto a tooth or tooth surface or into a chamber. The degassed fluid can comprise a tissue dissolving agent and/or a decalcifying agent. The degassed fluid can have a dissolved oxygen content less than about 9 mg/L, less than about 7 mg/L, less than about 5 mg/L, less than about 3 mg/L, less than about 1 mg/L, or some other value. A fluid for treatment can comprise a degassed fluid with a dissolved oxygen content less than about 9 mg/L, less than about 7 mg/L, less than about 5 mg/L, less than about 3 mg/L, less than about 1 mg/L, or some other value. The fluid can comprise a tissue dissolving agent and/or a decalcifying agent. For example, the degassed fluid can comprise an aqueous solution of less than about 6% by volume of a tissue dissolving agent and/or less than about 20% by volume of a decalcifying agent.

### Enhancing the Treatment of Teeth

The embodiments disclosed herein can advantageously remove undesirable or unhealthy materials from a tooth such that substantially all the unhealthy material is removed while inducing minimal or no discomfort and/or pain in the patient. For example, when activated by the clinician, a pressure wave generator can induce various fluidic effects that interact with the unhealthy material to be removed, even when the pressure wave generator is disposed at a position remote from the treatment region of the tooth, e.g., the region of the tooth that includes the unhealthy or undesirable material to be removed. The pressure wave generator can impart energy to a fluid that induces the relatively large-scale or bulk circulation or movement of liquid in a chamber and tooth, and that also generates pressure waves that propagate through the fluid and tooth. The generated fluid motion and pressure waves can magnify or enhance the properties of the fluid to enhance cleaning of the tooth. In some embodiments, the pressure wave generator can be used to obturate or fill the root canals and/or other treated regions of the tooth.

1. Chemistry of Various Treatment Fluids

In various embodiments, the treatment fluids described herein can comprise a treatment fluid that can be introduced into the tooth and the chamber to assist in removing unhealthy or undesirable materials from the tooth. The treatment fluids can be selected based on the chemical properties of the fluids when reacting with the undesirable or unhealthy material to be removed from the tooth. The treatment fluids disclosed herein can include any suitable fluid, including, e.g., water, saline, etc. Various chemicals can be added to treatment fluid for various purposes, including, e.g., tissue dissolving agents (e.g., NaOCl or bleach), disinfectants (e.g., chlorhexidine), anesthesia, fluoride therapy agents, ethylenediaminetetraacetic acid (EDTA), citric acid, and any other suitable chemicals. For example, any other antibacterial, decalcifying, disinfecting, mineralizing, or whitening solutions may be used as well. The clinician can supply the various fluids to the tooth in one or more treatment cycles, and can supply different fluids sequentially or simultaneously.

During some treatment cycles, bleach-based solutions (e.g., solutions including NaOCl) can be used to dissociate diseased tissue (e.g., diseased organic matter in the root canal) and/or to remove bacteria from the tooth. One example of a treatment solution comprises water or saline with 0.3% to 6% bleach (NaOCl). In some methods, tissue dissolution and dental deposit removal in the presence of bleach may not occur when the bleach concentration is less than 1%. In some treatment methods disclosed herein, tissue dissolution and dental deposit removal can occur at smaller (or much smaller) concentrations.

During other treatment cycles, the clinician can supply an EDTA-based solution to remove undesirable or unhealthy calcified material from the tooth. For example, if a portion of the tooth and/or root canal is shaped or otherwise instrumented during the procedure, a smear layer may form on the walls of the canal. The smear layer can include a semi-crystalline layer of debris, which may include remnants of pulp, bacteria, dentin, and other materials. Treatment fluids that include EDTA may be used to remove part or all of the smear layer, and/or calcified deposits on the tooth.

During yet other cycles, for example, the clinician may supply a treatment fluid that comprises substantially water. The water can be used to assist in irrigating the tooth before, during, and/or after the treatment. For example, the water can be supplied to remove remnants of other treatment fluids (e.g., bleach or EDTA) between treatment cycles. Because bleach has a pH that tends to be a base and because EDTA is an acid, it can be important to purge the tooth and chamber between bleach and EDTA treatments to avoid potentially damaging chemical reactions. Furthermore, the water can be supplied with a sufficient momentum to help remove detached materials that are disrupted during the treatment. For example, the water can be used to convey waste material from the tooth.

Various solutions may be used in combination at the same time or sequentially at suitable concentrations. In some embodiments, chemicals and the concentrations of the chemicals can be varied throughout the procedure by the clinician and/or by the system to improve patient outcomes. For example, during an example treatment procedure, the clinician can alternate between the use of water, bleach, and EDTA, in order to achieve the advantages associated with each of these chemicals. In one example, the clinician may begin with a water cycle to clean out any initial debris, then proceed with a bleach cycle to dissociate diseased tissue and bacteria from the tooth. A water cycle may then be used to remove the bleach and any remaining detached materials from the tooth. The clinician may then supply EDTA to the tooth to remove calcified deposits and/or portions of a smear layer from the tooth. Water can then be supplied to remove the EDTA and any remaining detached material from the tooth before a subsequent bleach cycle. The clinician can continually shift between cycles of treatment fluid throughout the procedure. The above example is for illustrative purposes only. It should be appreciated that the order of the cycling of treatment liquids may vary in any suitable manner and order.

Thus, the treatment fluids used in the embodiments disclosed herein can react chemically with the undesirable or unhealthy materials to dissociate the unhealthy materials from the healthy portions of the tooth. The treatment fluids can also be used to irrigate waste fluid and/or detached or delaminated materials out of the tooth. In some embodiments, the treatment solution (including any suitable composition) can be degassed, which may improve cavitation and/or reduce the presence of gas bubbles in some treatments. In some embodiments, the dissolved gas content can be less than about 1% by volume.

2. Enhancement of Cleaning Using Pressure Waves

As explained herein, a pressure wave generator can remove unhealthy materials from a tooth by propagating pressure waves through a propagation medium (e.g., the treatment fluid) to the treatment region, which can include one or more teeth and/or gums. Without being limited by theory, a few potential ways that the pressure waves remove undesirable materials are presented herein. Note that these principles, and the principles described above, may be generally applicable for each embodiment disclosed herein.

In some arrangements, cavitation may be induced by the generated pressure waves. Upon irradiation of a liquid (e.g., water or other treatment fluid) with high intensity pressure or pressure waves, acoustic cavitation may occur. The oscillation or the implosive collapse of small cavitation bubbles can produce localized effects, which may further enhance the cleaning process, e.g., by creating intense, small-scale localized heat, shock waves, and/or microjets and shear flows. Therefore, in some treatment methods, acoustic cavitation may be responsible for or involved in enhancing the chemical reactions, sonochemistry, sonoporation, soft tissue/cell/bacteria dissociation, delamination and breakup of biofilms.

For example, if the treatment liquid contains chemical(s) that act on a particular target material (e.g., diseased organic or inorganic matter, stains, caries, dental calculus, plaque, bacteria, biofilms, etc.), the pressure waves (acoustic field) and/or the subsequent acoustic cavitation may enhance the chemical reaction via agitation and/or sonochemistry. Indeed, the pressure waves can enhance the chemical effects that each composition has on the unhealthy material to be removed from the tooth. For example, with a bleach-based treatment fluid, the generated pressure waves can propagate so as to dissociate tissue throughout the entire tooth, including in the dentinal tubules and throughout tiny cracks and crevices of the tooth. As another example, with an EDTA-based treatment fluid, the generated pressure waves can propagate so as to remove the smear layer and/or calcified deposits from the tooth, including in the tubules and/or in tiny cracks and crevices formed in the tooth. With a water-based treatment fluid, the generated pressure waves can propagate so as to flush and/or irrigate undesirable materials from the tooth, including in tubules and tiny cracks and crevices. Accordingly, the generated pressure waves can enhance the removal of undesirable or unhealthy materials from the tooth by magnifying the chemical effects of whatever treatment fluid composition is used during a particular treatment cycle.

Furthermore, sonoporation, which is the process of using pressure waves and/or the subsequent acoustic cavitation to modify the permeability of the bacterial cell plasma membrane, may also expedite the chemical reaction that removes the microorganisms from the tooth. It should also be appreciated that generated pressure waves, and/or the subsequent acoustic cavitation of certain frequencies, may result in cellular and bacterial rupture and death (e.g., lysis) as well as removal of decayed and weakened dentin and enamel. The cellular and bacterial rupture phenomenon may kill bacteria which might otherwise reinfect the gingival pockets and/or the oral cavity.

Generated pressure waves and/or the subsequent acoustic cavitation may also loosen the bond of the structure of the unhealthy material (e.g., diseased tissue, calculus, biofilm, caries, etc.), and/or the pressure waves may dissociate the unhealthy material from the tooth. In some cases, pressure waves and/or acoustic cavitation may loosen the bond between the cells and the dentin and/or delaminate the tissue from the tooth. Furthermore, the pressure waves and/or the subsequent acoustic cavitation may act on decayed hard tissue (which may be relatively weak and loosely connected) through vibrations and/or shock waves, and/or the microjets created as a result of cavitation bubble implosion, to remove decayed hard tissue from other healthy portions of the tooth.

3. [0239] Enhancement of Cleaning Using Large-Scale Fluid Motion

In some arrangements, bulk fluid motion (e.g., fluid rotation, convection, planar flow, chaotic flow, etc.) can enhance the cleaning of unhealthy material from a diseased tooth. For example, the fluid motion generated in a chamber and/or tooth can impart relatively large momentum to the tooth, which can help dissociate and irrigate unhealthy materials from the tooth. Furthermore, the fluid motion can induce vortices and/or swirl in the tooth that can result in negative pressures (or low positive pressures) near the apical opening of the tooth. The resulting negative pressures at the apical opening can prevent or reduce an amount of material extruded through the apical opening and into the jaw of the patient. By preventing or reducing the amount of extruded material, the risk of infection can be lowered or eliminated, and patient outcomes can be substantially improved.

In addition, due to relatively short time scales of the chemical reaction processes between the fluid and the unhealthy materials as compared to that of diffusion mechanisms, a faster mechanism of reactant delivery such as “macroscopic” liquid circulation may be advantageous in some of the embodiments disclosed herein. For example, liquid circulation with a time scale comparable to (and preferably faster than) that of chemical reaction may help replenish the reactants at the chemical reaction front and/or may help to remove the reaction byproducts from the reaction site. The relatively large convective time scale, which may relate to effectiveness of the convection process, can be adjusted and/or optimized depending on, e.g., the location and characteristics of the source of circulation. Furthermore, it should be appreciated that the introduction of liquid circulation or other fluid motion generally does not eliminate the diffusion process, which may still remain effective within a thin microscopic layer at the chemical reaction front. Liquid circulation can also cause a strong irrigation effect at the treatment site (e.g. removing diseased tissue deep in the canal and/or tubules and small spaces and cracks of the tooth) and may therefore result in loosening and/or removing large and small pieces of debris from the treatment site.

In some arrangements, various properties can be adjusted to enhance bulk fluid motion and/or fluid circulation, e.g., fluid motion in the chamber. For example, the position of a fluid motion generator relative to the location of the treatment site can be adjusted. As explained herein, in some embodiments, a fluid motion generator is disposed such that the fluid motion generator passes a stream of liquid across an access opening. For example, the fluid motion generator can be disposed to induce fluid motion about an axis transverse to a central axis of a root canal, which can generate vortices that propagate throughout the canal. In some embodiments, the fluid motion can be generated about the central axis of the root canal, which can induce swirl motion in the root canal. The fluid flow over the access port or access opening of the tooth can be varied. For example, the momentum of the fluid can be varied to create the desired flow in the root canals. Furthermore, the angle of the fluid flow relative to the access port can be varied to control the apical pressure in the canals, e.g., to induce apical pressures that are more positive, more negative, etc. The geometry of the space surrounding the fluid motion generator and treatment site (e.g., the geometry of a coupling member) can also be varied. It should also be appreciated that circulation may be affected by the viscosity of the fluid and/or the mechanism of action of the fluid motion generator. For example, a fluid motion generator, such as a jet of liquid ejected through an inlet opening, a stirrer such as a propeller or a vibrating object, etc., can be selected to enhance fluid motion of the treatment fluid. In some aspects, the input power of the source of liquid circulation can also be adjusted, such as the source of a pump that drives a liquid jet in some embodiments.

4. Enhancement of Other Dental and Endodontic Procedures

In some embodiments, the pressure wave generators disclosed herein can enhance other dental and endodontic procedures. For example, after cleaning a tooth (e.g., a root canal inside the tooth, a carious region on or near an exterior surface of the tooth, etc.), the treatment region can be filled with an obturation or other filling material. In some embodiments, the filling material can be supplied to the treatment region as a flowable material and can be hardened to fill the treatment region (e.g., the cleaned root canal or carious region, etc.). In some embodiments, a pressure wave generator can be activated to supply the obturation material throughout the treatment region. For example, in some embodiments, pressure wave generators comprising electromagnetic generators can be used to fill a tooth by activating an electromagnetically responsive medium to produce pressure waves in the filling material.

For example, after a root canal procedure, the pressure wave generator can supply the flowable obturation material into the tooth and root canal. The large-scale fluid movement generated by a fluid motion generator can assist in propagating the filling material throughout relatively large spaces, such as the main root canal or canals, or through larger treated carious regions. For example, the fluid motion generator may introduce sufficient momentum such that the flowable filling material propagates throughout the canal space without introducing additional instrumentation into the tooth. For example, the bulk fluid motion of the filling material into the canal may be such that the clinician may not need to or desire to enlarge the canals. By reducing or eliminating canal enlargement, patient outcomes and pain levels can be improved. In some arrangements, the bulk fluid motion of the flowable obturation material can be generated at relatively low frequencies produced by the fluid motion generator.

In addition to generating large-scale or bulk fluid motion of the obturation material throughout the canal, the pressure wave generators disclosed herein can generate higher frequency perturbations to propagate the filling material into smaller cracks, spaces, and crevices in the tooth. For example, higher-frequency effects, such as acoustic cavitation, can assist in propagating the filler material throughout the tooth.

Accordingly, the pressure wave generators disclosed herein can enhance the filling of a treatment region such as a root canal, carious region of the tooth, etc. For example, the filling material can be propagated at a distance such that it flows into the treatment region from a remote pressure wave generator (which may be disposed outside the tooth). Large-scale or bulk fluid motion of the filling material can fill larger canal spaces or other treatment regions without further enlargening the treatment region. Smaller-scale and/or higher frequency agitation by the pressure wave generator can propagate the filling material into smaller cracks and spaces of the tooth. By filling substantially all the cleaned spaces of the tooth, the disclosed methods can improve patient outcomes relative to other methods by reducing the risk of infection in spaces unfilled by the filling material.

5. Additional Enhancements

The embodiments disclosed herein can advantageously remove undesirable or unhealthy materials from a tooth such that substantially all the unhealthy material is removed while inducing minimal or no discomfort and/or pain in the patient. For example, when activated by the clinician, a pressure wave generator can induce various fluidic effects that interact with the unhealthy material to be removed, even when the pressure wave generator is disposed at a position remote from the treatment region of the tooth, e.g., the region of the tooth that includes the unhealthy or undesirable material to be removed. The pressure wave generator can impart energy to a fluid that induces the relatively large-scale or bulk circulation or movement of liquid in the chamber and tooth, and that also generates pressure waves that propagate through the fluid and tooth. The generated fluid motion and pressure waves can magnify or enhance the properties of the fluid to enhance cleaning of the tooth. In some embodiments, the pressure wave generator can be used to obturate or fill the root canals and/or other treated regions of the tooth.

It is believed, although not required, that some or all of the effects described herein may be at least in part responsible for advantageous effects, benefits, or results provided by various implementations of the treatment methods and systems described herein. Accordingly, various embodiments of the systems disclosed herein can be configured to provide some or all of these effects.

In the following description, unless a different meaning is indicated, the following terms have their ordinary and customary meaning. For example, a chemical reaction front may generally refer to an interface between the tissue and the solution which contains a chemical such as a tissue dissolving agent. Tissue may refer to all types of cells existing in the tooth as well as bacteria and viruses. Calcified tissue may refer to calcified pulp, pulp stones, and tertiary dentin. Bubbles includes but is not limited to bubbles created due to a chemical reaction, dissolved gas remaining in the fluid after degassing (if used) and released as bubbles in the fluid, and any bubbles which are introduced into the tooth due to imperfect sealing.

Tissue cleaning treatments may utilize one or more of the physicochemical effects described herein to clean and remove tissue and/or calcified tissue from a tooth chamber. In some cleaning treatments, the combination of (1) acoustic or pressure waves (e.g., generation of acoustic cavitation), (2) circulation of fluid in the chamber (e.g., macroscopic eddies and flows), and (3) chemistry (e.g., use of a tissue dissolving agent, use of degassed fluids) can provide highly effective cleaning. Accordingly, certain embodiments of the systems disclosed herein utilize a pressure wave generator to generate the acoustic waves, a fluid platform (e.g., fluid retainer) to retain treatment fluid in the tooth chamber and to enable circulation of the treatment fluid, and a treatment fluid that is degassed or includes a chemical agent such as a tissue dissolving agent.

6. Pressure Waves

A pressure wave generator can be used to generate pressure waves that propagate through the fluid in the chamber (and the tooth). Upon irradiation of a fluid with high intensity pressure waves (e.g., broadband frequencies), acoustic cavitation may occur. As has been described herein, the implosive collapse of the cavitation bubbles can produce intense local heating and high pressures with short lifetimes. Therefore, in some treatment methods, acoustic cavitation may be responsible for or involved in enhancing chemical reactions, sonochemistry, sonoporation, tissue dissociation, tissue delamination, as well as removing the bacteria and/or the smear layer from the root canals and tubules.

Sonoporation is the process of using an acoustic field to modify the permeability of the cell plasma membrane. This process may greatly expedite the chemical reaction. It may be advantageous if the acoustic field has a relatively broad bandwidth (e.g., from hundreds to thousands of kHz). Some frequencies (e.g., low frequency ultrasound) may also result in cellular rupture and death (e.g., lysis). This phenomenon may kill bacteria which might otherwise reinfect the tooth. Acoustic waves and/or acoustic cavitation may loosen the bond between cells and/or may dissociate the cells. Acoustic waves and/or acoustic cavitation may loosen the bond between cells and dentin and/or delaminate the tissue from the dentin.

For removing calcified tissue, acoustic waves may induce sonochemistry and microscopic removal of calcified structures due to shock waves and/or microjets created as a result of cavitation bubble implosion. Pressure or acoustic waves may break microscopic calcified structures through structural vibrations. If a chemical (e.g., a chelating agent such as, e.g., EDTA) is used for this procedure, the acoustic waves may enhance the chemical reaction.

Certain properties of the system can be adjusted to enhance the effects of the acoustic waves. For example, properties of the fluid including, e.g., surface tension, boiling or vapor temperature, or saturation pressure can be adjusted. A degassed fluid with a reduced dissolved gas content can be used, which may reduce the energy loss of acoustic waves that may be generated by hydrodynamic cavitation or any other sources. The fluid can be degassed, which may help preserve the energy of the acoustic waves and may increase the efficiency of the system.

7. Fluid Circulation

Some treatment systems and methods use diffusion and/or acoustically enhanced diffusion of reactants and byproducts to and away from the chemical reaction front. However, due to the relatively short time scale of the reaction process, a faster mechanism of reactant delivery such as “macroscopic” fluid motion, circulation, convection, vorticity, or turbulence may be advantageous in some of the embodiments disclosed herein. For example, fluid inflow into the tooth chamber may induce a macroscopic circulation in the pulp cavity. As described herein, fluid motion generator, such as a liquid jet device, may induce circulation, for example, in the case of a liquid jet device, as the jet and/or spray enter the chamber. Other fluid motion generators can produce fluid circulation via their interaction with ambient fluid (e.g., via localized heating of the fluid, which may induce convection currents and circulation).

Fluid circulation with a time scale comparable to (and preferably faster than) that of chemical reaction may help replenish the reactants at a chemical reaction front and/or may help to remove reaction byproducts from the reaction site. The convective time scale, which may relate to effectiveness of the convection or circulation process, can be adjusted depending on, e.g., the location and characteristics of the source of circulation. The convective time scale is approximately the physical size of the chamber divided by the fluid speed in the chamber. Introduction of circulation generally does not eliminate the diffusion process, which may still remain effective within a thin microscopic layer at the chemical reaction front. Fluid circulation may create flow-induced pressure oscillations inside the root canal which may assist in delaminating, loosening, and/or removing larger pieces tissue from the root canal.

For removing calcified tissue, fluid circulation may create flow-induced pressure oscillations inside the root canal which may assist in removing larger pieces of calcified structures from the root canal.

Certain properties of the system can be adjusted to enhance the effects of the circulation in the tooth. For example, the location of the source of circulation inside the tooth, the source flow characteristics such as shape (e.g. planar vs. circular jets) or velocity and/or direction of a fluid stream, and the fluid kinematic viscosity may be adjusted. The circulation may also be effected by the anatomy of the tooth or the canal orifice or root canal size. For example, a narrow root canal with constrictions may have a lower solution replenishment rate than a wide canal with no constrictions. If the source of convection/circulation is placed near the pulp chamber floor, a tooth with a smaller pulp chamber may have stronger circulation than one with a larger pulp chamber. Convection-induced pressure exerted at the periapical region of the tooth may be controlled to reduce or avoid extrusion of the treatment fluid into the periapical tissues. Large magnitude vacuum or low pressure in the tooth may cause discomfort in some patients. Thus, the properties of the coupling member (e.g., vents, sponges, flow restrictors, etc.) can be adjusted to provide a desired operating pressure range in the chamber and/or tooth.

8. Chemistry

As explained herein, various reaction chemistries can be adjusted or designed to improve the cleaning process. For example, to enhance the dissolution of organic tissue, a tissue dissolving agent (e.g., a mineralization therapy agent, EDTA, sodium hypochlorite—NaOCl) can be added to the treatment liquid. The agent may react with various components at the treatment site. In some cases, tissue dissolution may be a multi-step process. The agent may dissolve, weaken, delaminate or dissociate organic and/or inorganic matter, which may result in better patient outcomes. The chemical reaction can modify the physical characteristics of the treatment solution locally (e.g., reducing the local surface tension via saponification), which may assist in the penetration of the treatment liquid into gaps and small spaces in the treatment sites or to remove bubbles formed during the chemical reaction. A tissue dissolving agent (e.g., sodium hypochlorite or bleach) may be added to the treatment fluid to react with tissue. Tissue dissolution may be a multi-step and complex process. Dissolution of sodium hypochlorite in water can include a number of reactions such as, e.g., the sodium hypochlorite (bleach) reaction, a saponification reaction with triglycerides, an amino acid neutralization reaction, and/or a chloramination reaction to produce chloramine. Sodium hypochlorite and its by-products may act as dissolving agents (e.g. solvents) of organics, fats, and proteins; thereby, degrading organic tissue in some treatments.

Sodium hypochlorite may exhibit a reversible chemical equilibrium based on the bleach reaction. Chemical reactions may occur between organic tissue and sodium hypochlorite. For example, sodium hydroxide can be generated from the sodium hypochlorite reaction and can react with organic and fat (triglycerides) molecules to produce soap (fatty acid salts) and glycerol (alcohol) in the saponification reaction. This may reduce the surface tension of the remaining solution. Sodium hydroxide can neutralize amino acids forming amino acid salts and water in the amino acid neutralization reaction. Consumption of sodium hydroxide can reduce the pH of the remaining solution. Hypochlorous acid, a substance that can be present in sodium hypochlorite solution, can release chlorine that can react with amino groups of proteins and amino acids to produce various chloramines derivatives. For example, hypochlorous acid can react with free amino acids in tissue to form N-chloro amino acids which can act as strong oxidizing agents that may have higher antiseptic activity than hypochlorite.

Chemical(s) in the fluid, depending on their type, may affect the surface tension of the solution, which in turn may modify the cavitation phenomenon. For example, solution of an inorganic chemical such as, e.g., sodium hypochlorite in water, may increase the ion concentration in the solution which may increase the surface tension of the solution, which may result in stronger cavitation. In some cases, the magnitude of a cavitation inception threshold may increase with increasing surface tension, and the cavitation inducing mechanism (e.g., a pressure wave generator) may be sufficiently intense to pass the threshold in order to provide inception of cavitation bubbles. It is believed, but not required, that once the cavitation threshold is passed, increased surface tension may result in stronger cavitation. Reducing the dissolved gas content of a fluid (e.g., via degassing) can increase the surface tension of the fluid and also may result in stronger cavitation. Addition of chemicals, agents, or substances (e.g., hydroxyl functional groups, nanoparticles, etc.) to the treatment may increase the efficiency of conversion of a pressure wave into cavitation, and such chemoacoustic effects may be desirable in some treatment procedures.

In some methods, a chemical, such as sodium hypochlorite, may cause saponification. The removal of bubbles created or trapped inside the root canals (or tubules) may be accelerated due to local reduction of surface tension at the chemical reaction front as a result of saponification. Although in some methods it may be desirable to have a relatively high surface tension at the pressure wave source (e.g. inside the pulp chamber), inside the canals it may be beneficial to have locally reduced surface tension to accelerate bubble removal. This phenomenon may happen as tissue dissolving agent(s) react with the tissue. For example, sodium hypochlorite can act as a solvent degrading fatty acids, transforming them into fatty acid salts (soap) and glycerol (alcohol) that can reduce the surface tension of the remaining solution at the chemical reaction front.

A number of variables or factors may be adjusted to provide effective cleaning. For example, each chemical reaction has a reaction rate determining the speed of reaction. The reaction rate may be dependent on several parameters including temperature. The concentration of reactants can be a factor and may affect the time for the reaction to complete. For instance, a 5% sodium hypochlorite solution generally may be more aggressive than a 0.5% sodium hypochlorite solution and may tend to dissolve tissue faster.

The refreshment rate of reactants may be affected by some or all of the following. Bubbles may form and stay at the chemical reaction front (e.g., due to surface tension forces) and may act as barriers at the chemical reaction front impeding or preventing fresh reactants from reaching the reaction front. Accordingly, circulation of the treatment fluid can help remove the bubbles and the reaction byproducts, and may replace them with fresh treatment fluid and fresh reactants. Thus, use of an embodiment of the fluid platform that can provide fluid circulation in the tooth chamber advantageously may improve the cleaning process.

Heat may increase the chemical reaction rate and may be introduced through a variety of sources. For example, the treatment solution may be preheated before delivery to the tooth chamber. Cavitation, exothermic chemical reactions, or other internal or external dissipative sources may produce heat in the fluid, which may enhance, sustain, or increase reaction rates.

Sonication of the fluid may increase chemical reaction rates or effectiveness. For example, upon irradiation of a fluid (e.g., water) with high intensity pressure waves (including, e.g., sonic or ultrasonic waves, or broad spectrum acoustic power produced by a liquid jet) acoustic cavitation may occur. The implosive collapse of the cavitation bubbles can produce intense local heating and high pressures with short lifetimes. Experimental results have shown that at the site of the bubble collapse, the temperature and pressure may reach around 5000 K and 1000 atm, respectively. This phenomenon, known as sonochemistry, can create extreme physical and chemical conditions in otherwise cold liquids. Sonochemistry, in some cases, has been reported to enhance chemical reactivity by as much as a million fold. In cases where acoustic cavitation does not occur (or occurs at a relatively low amplitude), the vibration of reactants, due to the pressure waves, may enhance the chemical reaction as it assists in replacing the byproducts by fresh reactants.

For removing calcified tissue, a decalcifying agent (e.g., an acid such as, e.g., EDTA or citric acid) may be added to the treatment fluid. The decalcifying agent may remove calcium or calcium compounds from the tooth dentin. The substances remaining after treatment with the decalcifying agent may be relatively softer (e.g., gummy) than prior to treatment and more easily removable by the fluid circulation and acoustic waves.

Reference throughout this specification to “some embodiments” or “an embodiment” means that a particular feature, structure, element, act, or characteristic described in connection with the embodiment is included in at least one embodiment. Thus, appearances of the phrases “in some embodiments” or “in an embodiment” in various places throughout this specification are not necessarily all referring to the same embodiment and may refer to one or more of the same or different embodiments. Furthermore, the particular features, structures, elements, acts, or characteristics may be combined in any suitable manner (including differently than shown or described) in other embodiments. Further, in various embodiments, features, structures, elements, acts, or characteristics can be combined, merged, rearranged, reordered, or left out altogether. Thus, no single feature, structure, element, act, or characteristic or group of features, structures, elements, acts, or characteristics is necessary or required for each embodiment. All possible combinations and subcombinations are intended to fall within the scope of this disclosure.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

The foregoing description sets forth various example embodiments and other illustrative, but non-limiting, embodiments of the inventions disclosed herein. The description provides details regarding combinations, modes, and uses of the disclosed inventions. Other variations, combinations, modifications, equivalents, modes, uses, implementations, and/or applications of the disclosed features and aspects of the embodiments are also within the scope of this disclosure, including those that become apparent to those of skill in the art upon reading this specification. Additionally, certain objects and advantages of the inventions are described herein. It is to be understood that not necessarily all such objects or advantages may be achieved in any particular embodiment. Thus, for example, those skilled in the art will recognize that the inventions may be embodied or carried out in a manner that achieves or optimizes one advantage or group of advantages as taught herein without necessarily achieving other objects or advantages as may be taught or suggested herein. Also, in any method or process disclosed herein, the acts or operations making up the method or process may be performed in any suitable sequence and are not necessarily limited to any particular disclosed sequence.

1. An apparatus for treating a tooth, the apparatus comprising:
a treatment fluid supply configured to supply a treatment fluid to a treatment region of the tooth; and
a pressure wave generator configured to generate pressure waves in the treatment fluid, the pressure wave generator comprising:
a volume housing an electromagnetically responsive material;
a diaphragm comprising a first side and a second side, the first side exposed to the volume, the second side exposed to the treatment fluid, the diaphragm being movable such that movement of the electromagnetically responsive material within the volume causes movement of the diaphragm; and
an electromagnetic generator coupled to the volume, the electromagnetic generator configured to generate electromagnetic energy;
wherein the electromagnetically responsive material is responsive to the electromagnetic energy generated by the electromagnetic generator so as to cause the movement of the diaphragm.

2. The apparatus of claim 1, wherein the electromagnetically responsive material comprises a ferrofluid.

3. The apparatus of claim 1, wherein the electromagnetic generator comprises a magnetic field generator.

4. (canceled)

5. (canceled)

6. (canceled)

7. The apparatus of claim 1, wherein the electromagnetic generator is positioned within a flow path of the treatment fluid within the treatment fluid supply.

8. (canceled)

9. (canceled)

10. (canceled)

11. The apparatus of claim 1, further comprising a controller configured to control operation of the electromagnetic generator.

12. The apparatus of claim 11, wherein the controller is configured to send control signals to the electromagnetic generator, the control signals selected to cause the electromagnetic generator to generate electromagnetic energy which causes a response in the electromagnetically responsive material to produce acoustic waves in the treatment fluid having a predetermined acoustic signature.

13.-18. (canceled)

19. An apparatus for treating a tooth, the apparatus comprising:
a pressure wave generator comprising an electromagnetic element configured to convert electromagnetic energy to acoustic waves in a fluid by way of an electromagnetically responsive medium; and
a controller configured to send control signals to the electromagnetic element, the control signals selected to cause the electromagnetic element to generate electromagnetic energy that produces acoustic waves in the fluid having a predetermined acoustic signature.

20. (canceled)

21. (canceled)

22. (canceled)

23. The apparatus of claim 19, wherein the electromagnetically responsive medium comprises a plurality of ferrous particles.

24.-34. (canceled)

35. The apparatus of claim 19, further comprising a sensor configured to measure an acoustic signature of acoustic waves produced during a treatment procedure.

36. The apparatus of claim 35, wherein the controller is configured to adjust the control signals based on the measured acoustic signature of the acoustic waves produced during the treatment procedure.

37. The apparatus of claim 36, wherein the controller is configured to compare the measured acoustic signature to the predetermined acoustic signature and adjust the control signals based on the comparison between the measured acoustic signature and the predetermined acoustic signature.

38.-108. (canceled)

109. An apparatus for treating a treatment region of a tooth, the apparatus comprising:
a fluid platform comprising a chamber to be positioned against the tooth, the chamber shaped to retain treatment fluid;
a fluid passage in fluid communication with the chamber via an opening at a distal end of the fluid passage, the fluid passage in fluid communication with the chamber through the opening; and
a pressure wave generator positioned to generate pressure waves in the treatment fluid at a location within the fluid passage proximal of the opening and outside of the tooth, the generated pressure waves being transmitted through the treatment fluid in the chamber to the treatment region of the tooth.

110. The apparatus of claim 109, wherein the pressure wave generator comprises an electromagnetic generator.

111. The apparatus of claim 110, wherein the electromagnetic generator is configured to convert electromagnetic energy to pressure waves in the treatment fluid by way of an electromagnetically responsive medium.

112. The apparatus of claim 111, further comprises a controller configured to send control signals to electromagnetic generator, the control signals selected to cause the electromagnetic generator to generate electromagnetic energy that produces acoustic waves in the treatment fluid having a predetermined acoustic signature.

113. The apparatus of claim 109, wherein the electromagnetic generator comprises a magnetic field generator.

114. The apparatus of claim 111, wherein the electromagnetically responsive medium comprises a plurality of ferrous particles.

115. The apparatus of claim 114, wherein the plurality of ferrous particles are suspended within the treatment fluid.

116. The apparatus of claim 115, wherein the electromagnetic generator is configured to generate electromagnetic energy that causes movement of the ferrous particles in the fluid which produces waves in the fluid having the predetermined acoustic signature.

117. The apparatus of claim 111, further comprising:
a volume housing the electromagnetically responsive medium; and
a diaphragm comprising a first side and a second side, the first side exposed to the volume, the second side exposed to the fluid, the diaphragm being movable such that movement of the electromagnetically responsive medium within the volume causes movement of the diaphragm.

118. The apparatus of claim 117, further comprising a deformable material separating the volume housing the electromagnetically responsive material and the electromagnetic generator.

119. (canceled)

120. (canceled)

121. The apparatus of claim 109, wherein the pressure wave generator comprises a liquid jet apparatus.

122. The apparatus of claim 121, wherein the liquid jet apparatus comprises a nozzle configured to produce a high velocity liquid jet.

123. The apparatus of claim 121, wherein the pressure wave generator further comprises an impingement plate.

124. The apparatus of claim 109, wherein the pressure wave generator comprises a sonic, ultrasonic, or megasonic device.

125. The apparatus of claim 109, wherein the pressure wave generator comprises a mechanical stirrer.

126. The apparatus of claim 109, wherein the pressure wave generator comprises a laser device configured to propagate optical energy within the treatment fluid.

127. The apparatus of claim 109, further comprising a fluid motion generator configured to generate bulk fluid motion in the treatment fluid.

128. The apparatus of claim 127, wherein the fluid motion generator is located between the pressure wave generator and the opening.

129. The apparatus of claim 127, wherein the fluid motion generator is located within the chamber.

130. The apparatus of claim 109, wherein the pressure wave generator is configured to generate bulk fluid motion in the treatment fluid.

131. The apparatus of claim 109, wherein the pressure wave generator is positioned downstream of the chamber.

132. The apparatus of claim 109, wherein the fluid passage is a fluid outlet line configured to evacuate fluid from the treatment region.

133. The apparatus of claim 132, further comprising a vent disposed along the outlet line, the vent being exposed to ambient air.

134. The apparatus of claim 109, wherein the pressure wave generator is positioned upstream of the chamber.

135. The apparatus of claim 109, wherein the fluid passage is a fluid inlet line.

136. (canceled)

137. The apparatus of claim 109, wherein the generated pressure waves have a broadband power spectrum and multiple frequencies.

138.-182. (canceled)
